(19) United States
(12) Patent Application Publication
(10) Pub. No.: US 2025/0100994 A1
(43) Pub. Date: Mar. 27, 2025

(54) CDK12/13 COVALENT INHIBITORS OR PHARMACEUTICAL COMPOSITION THEREOF, AND USES THEREOF

(71) Applicants: The Regents of the University of Michigan, Ann Arbor, MI (US); Shanghai Institute of Organic Chemistry, Chinese Academy of Sciences, Shanghai (CN)

(72) Inventors: Arul M. Chinnaiyan, Ann Arbor, MI (US); George X. Wang, Ann Arbor, MI (US); Yu Chang, Ann Arbor, MI (US); Jean Tien, Ann Arbor, MI (US); Ke Ding, Shanghai (CN); Jianzhang Yang, Shanghai (CN); Weixue Huang, Shanghai (CN); Zhen Wang, Shanghai (CN)

(21) Appl. No.: 18/899,125

(22) Filed: Sep. 27, 2024

Related U.S. Application Data
(60) Provisional application No. 63/540,752, filed on Sep. 27, 2023.

(51) Int. Cl. C07D 401/12; A61K 31/44; A61K 31/4418; A61K 31/4439; A61K 31/4545; A61K 31/496; A61K 31/506; A61K 31/5377; C07D 213/84; C07D 239/42; C07D 401/14; C07D 405/12; C07D 405/14; C07D 413/14; C07D 487/08

(52) U.S. Cl. CPC C07D 401/12; A61K 31/44; A61K 31/4418; A61K 31/4439; A61K 31/4545; A61K 31/496; A61K 31/506; A61K 31/5377; C07D 213/84; C07D 239/42; C07D 401/14; C07D 405/12; C07D 405/14; C07D 413/14; C07D 487/08

(57) ABSTRACT

The present invention relates to a method for preparing a class of novel CDK12/13 covalent inhibitors or a pharmaceutical composition thereof, and use thereof. The class of novel CDK12/13 covalent inhibitors of the present invention have a structure shown in formula (I). Such compounds can serve as protein kinase inhibitors, can effectively and highly selectively inhibit the CDK12/13 protein kinase activity, and can inhibit the proliferation, migration, and invasion of various tumor cells.

### CROSS-REFERENCE TO RELATED APPLICATIONS

This application claims priority to U.S. Provisional Patent Application No. 63/540,752, filed on Sep. 27, 2023, the entire contents of which are hereby incorporated by reference.

### STATEMENT REGARDING FEDERALLY SPONSORED RESEARCH AND DEVELOPMENT

This invention was made with government support under CA231996 and CA186786 awarded by the National Institutes of Health, and under W81XWH-21-1-0458 awarded by the Defense Health Agency, Medical Research and Development Branch. The government has certain rights in the invention.

### TECHNICAL FIELD

The present invention belongs to the chemical pharmaceutical field, and particularly relates to a class of novel CDK12/13 covalent inhibitors or a pharmaceutical composition thereof, and use thereof.

### BACKGROUND ART

Cyclin dependent kinases (CDKs) are a class of serine/threonine kinases. The CDKs play an important role in the control of cell division, and regulate transcription in response to a variety of intracellular and extracellular signals. 20 CDKs and 29 cyclins in mammals have been identified and reported. The CDKs require binding to their one or more corresponding cyclins for stabilization, activation and downstream phosphorylation. The CDKs in mammals are divided into three cell cycle-related subfamilies and six transcriptional subfamilies.

CDK12/13 (Cyclin-dependent kinase 12/13) is a member of a cyclin-dependent kinase family (CDKs) of serine/threonine protein kinases, and forms a complex with Cyclin K to realize biological functions. The CDK12/13 comprises 1490/1512 amino acids, respectively, and shares 46% homology. The kinase domain consists of 300 amino acids with as high as 92% homology. Compared to other transcribed CDKs, the CDK12/13 contains an additional N-terminal arginine/serine-rich (RS) motif, which is commonly found in proteins involved in pre-mRNA splicing. A proline-rich motif (PRIM) is also found at the N- and C-termini, and is likely to serve as a binding site for SH3, WW, or a protein containing an actin-binding protein (profilin) domain. The CDK12/13 forms a complex together with the Cyclin K by phosphorylating a C-terminal domain (CTD) of RNA polymerase II (RNA Pol II). The CTD is a highly repeated sequence composed of seven amino acids (YSPTSPS). CTD comprises a 52-repeat unit in human. The CDK12/13 mainly phosphorylates Ser at position 2, to regulate transcription and post-transcriptional mRNA processing. Genetic studies show that the CDK12 promotes the transcription of a full-length gene product by inhibiting the cleavage of an intronic polyadenylation site, and many homologous recombination repair genes (such as: BRCA1/2, ATM, ATR, FANCD2, and FANCI) comprise more intronic polyadenylation sites. Therefore, the expression of these genes is more sensitive to the loss or inhibition of the CDK12. DNA double strand breaks are extremely toxic. Homologous recombination is the most accurate and error-free way to repair the double strand breaks, and is crucial in the process of DNA damage repair. Therefore, inhibition of the CDK12/CDK13 can induce cells to produce DNA damage caused by homologous recombination deficiency (HRD), thereby killing cells. The combined use of a CDK12/13 inhibitor and other drugs has a synergistic effect to promote the killing of cancer cells, overcome drug resistance, and reduce the occurrence of drug-resistant strains. Further, the CDK12 has synthetic lethal interactions with MYC, EWS/FLI fusions and PARPi. Different proportions of CDK12 mutations have been reported in castration-resistant prostate cancer, high-grade serous ovarian cancer, etc., most of which are nonsense mutations or inactivating mutations. The CDK12 mutation is not a necessary condition for its use as a potential target for cancer therapy, and modulation of the expression of proteins related to DNA damage repair and synthetic lethal interactions provides an opportunity for its use as an anticancer target. The CDK12 has great potentials in disease treatment, and can be used as a biomarker of carcinogenesis, thereby attracting increasing interests of scientists in recent years.

### SUMMARY

In view of this, the present invention provides use of a class of novel CDK12/13 covalent inhibitors or a pharmaceutical composition thereof as a CDK12/13 inhibitor. Such compounds can effectively and highly selectively inhibit a CDK12/13 protein kinase, and can inhibit the proliferation, migration, and invasion of various tumor cells.

A first aspect of the present invention provides a CDK12/13 covalent inhibitor having a structure of formula (I), or a pharmaceutically acceptable salt thereof, or a pharmaceutical composition thereof, or a prodrug molecule thereof:

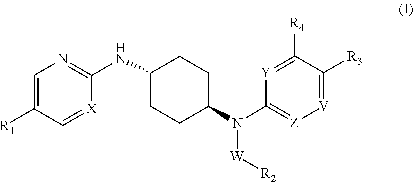

wherein X, Y, and Z are each independently selected from the group consisting of: N or CR₅;
R₅ is selected from the group consisting of: hydrogen, halogen, cyano, hydroxyl, amino, halomethyl, halomethoxy, haloethyl, haloethoxy, C₁-C₆ alkyl, C₁-C₆ alkoxy, C₃-C₈ cycloalkyl, C₃-C₈ cycloalkoxy, or C₁-C₆ alkyl-substituted amino;
R₁ is selected from the group consisting of: H, cyano, halogen, halomethyl, halomethoxy, haloethoxy, haloethyl, C₁-C₆ alkyl, C₃-C₈ cycloalkyl, C₁-C₆ alkoxy, or C₃-C₈ cycloalkoxy;
W is selected from the group consisting of: a chemical bond,

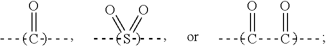

R₂ is selected from the group consisting of: —NHR₆, —OR₆, —CH(R₈)R₆, or H;
R₆ is selected from the group consisting of: —(C(R₈)R₇)R₉ or —(CH₂)ₙR₉; wherein n is selected from: 0, 1, or 2;
R₇ and R₈ are each independently selected from the group consisting of: hydrogen, halogen, cyano, methyl, halomethyl, methoxy, halomethoxy, ethyl, haloethyl, ethoxy, haloethoxy, hydroxyl, amino, or 3-8-membered heterocyclic ring containing m heteroatoms, wherein m is selected from: 1, 2, or 3, and the heteroatoms are selected from O, S, or N; or R₇ and R₈ form, together with the C atom to which they are attached, 3-7 heterocyclic rings containing n heteroatoms, wherein n is selected from: 1, 2, or 3, and the heteroatoms are selected from: O, N, or S;
R₉ is selected from the group consisting of:
1) cyano, C₁-C₅ alkyl, halogenated C₁-C₄ alkyl, C₁-C₄ alkoxy, C₃-C₁₀ cycloalkyl, substituted or unsubstituted 3-8-membered aromatic ring or saturated ring containing O, S, or N, or 8-12-membered fused ring, spiro ring, or bridged ring containing n heteroatoms; wherein n is selected from: 1, 2, or 3, and the heteroatom is selected from: O, N, or S; or
2)

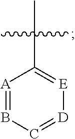

wherein A, B, C, D, and E are each independently selected from the group consisting of: CH, N, or CR₁₀;
R₁₀ is selected from the group consisting of: halogen, cyano, hydroxyl, amino, nitro, C₁-C₃ alkyl, halogenated C₁-C₃ alkyl, C₁-C₄ alkoxy, halogenated C₁-C₄ alkoxy, or C₃-C₈ cycloalkyl;
V is N or CR₃;
each R₃ is independently selected from the group consisting of: H, halogen, cyano, hydroxyl, amino, C₁-C₃ alkyl, halogenated C₁-C₃ alkyl, C₁-C₃ alkoxy, halogenated C₁-C₃ alkoxy, C₃-C₈ cycloalkyl, —(CH₂)ₘR₁₁, —NH(CH₂)ₘR₁₁, —NR₁₄(CH₂)ₘR₁₁, or —O(CH₂)ₘR₁₁; substituted or unsubstituted 3-8-membered heterocyclic ring containing m heteroatoms, wherein m is selected from: 1, 2, and 3, and the heteroatom is selected from: O, S, or N; or 8-12-membered fused ring, spiro ring, or bridged ring containing n heteroatoms, wherein n is selected from: 1, 2, or 3, the heteroatom is selected from: O, N, or S; and at least one R₃ is not H;
R₁₁ is selected from the group consisting of: C₁-C₆ alkyl or NR₁₂R₁₃; wherein R₁₂ and R₁₃ are each independently selected from the group consisting of: H, C₁-C₈ alkyl, —(CH₂)ₘNR₁₄R₁₅, or —(CH₂)ₙCR₁₄R₁₅R₁₆, or R₁₂ and R₁₃ form, together with a nitrogen atom to which they are attached, a substituted or unsubstituted heteroatom-containing monocyclic ring, fused ring, spiro ring, or bridged ring;
R₁₄, R₁₅, and R₁₆ are each independently selected from the group consisting of: H or C₁-C₈ alkyl, or R₁₄ and R₁₅ form, together with a nitrogen atom or carbon atom to which they are attached, a substituted or unsubstituted monocyclic ring, fused ring, spiro ring, or bridged ring containing from 0 to 3 heteroatoms;
m and n are independently selected from: an integer between 0 and 8;
R₄ is selected from the group consisting of: H,

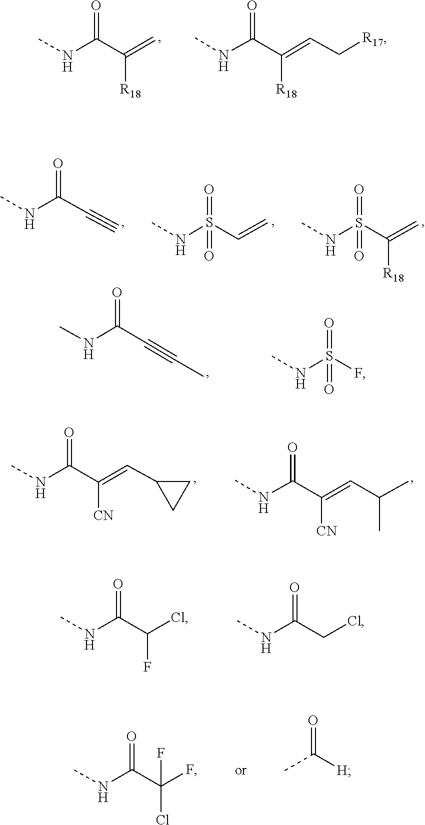

R₁₇ is selected from the group consisting of: hydrogen, trifluoromethyl,

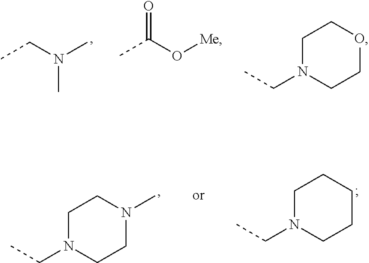

R₁₈ is selected from the group consisting of: hydrogen or fluorine; and unless otherwise specified, the “substituted” refers to the replacement of one or more hydrogen atoms on a group with a substituent selected from the group consisting of: halogen, oxo, unsubstituted or halogenated C1-C6 alkyl, unsubstituted or halogenated C2-C6 alkenyl, unsubstituted or halogenated C2-C6 alkynyl, unsubstituted or halogenated C1-C6 alkoxy, unsubstituted or halogenated C1-C6 acyl, unsubstituted or halogenated C1-C6 amido, unsubstituted or halogenated C₁-C₆ alkylamino, unsubstituted or halogenated C1-C6 alkyl-hydroxyl, unsubstituted or halogenated C3-C6 alkyl, or unsubstituted or halogenated 4-8-membered heterocyclyl.

In another preferred example, X, Y, and Z are each independently selected from: N or CR₅;
R₅ is selected from the group consisting of: hydrogen, halogen, cyano, halomethyl, or halomethoxy.

In another preferred example, R₁ is selected from the group consisting of: H, cyano, halogen, halomethyl, halomethoxy, haloethoxy, haloethyl, C₁-C₆ alkyl, C₃-C₈ cycloalkyl, or C₁-C₆ alkoxy.

In another preferred example, R₁ is selected from the group consisting of: H, cyano, halogen, or halomethyl.

In another preferred example, W is selected from:

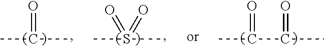

In another preferred example, R₂ is selected from the group consisting of: —NHR₆;
R₆ is selected from the group consisting of: —(C(R₈)R₇)R₉;
R₇ and R₈ are each independently selected from the group consisting of: hydrogen, halogen, cyano, methyl, halomethyl, methoxy, or halomethoxy; and
R₉ is each independently selected from the group consisting of: phenyl, halophenyl, pyridyl, pyrimidyl, isopropyl, tert-butyl, trifluoromethyl, difluoromethyl, cyano, pyrrolyl, N-methylpyrrolyl, N-methylimidazolyl, N-methylpyrazolyl, imidazolyl, furyl, thienyl, pyrazolyl, isoxazolyl, oxazolyl, halogenated C₁-C₄ alkyl, C₁-C₄ alkoxy, C₁-C₄ alkyl, C₃-C₇ cycloalkyl, or C₃-C₇ epoxyalkyl.

In another preferred example, the R₃ is selected from the group consisting of: H, halogen, cyano, hydroxyl, amino, C₁-C₃ alkyl, halogenated C₁-C₃ alkyl, C₁-C₃ alkoxy, halogenated C₁-C₃ alkoxy, C₃-C₈ cycloalkyl, C₃-C₈ cycloalkoxy, or 4-8-membered heterocyclyl (including monocyclic ring, condensed ring, or bridged ring).

In another preferred example, each R₃ is independently selected from the group consisting of: H, halogen, cyano, hydroxyl, amino, C₁-C₃ alkyl, halogenated C₁-C₃ alkyl, C₁-C₃ alkoxy, halogenated C₁-C₃ alkoxy, C₃-C₈ cycloalkyl, C₃-C₈ cycloalkoxy,

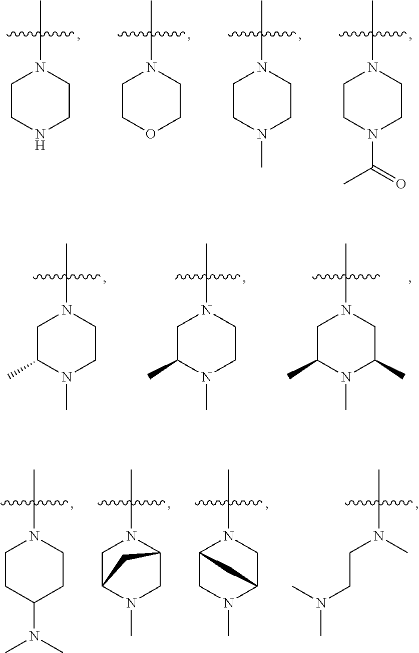

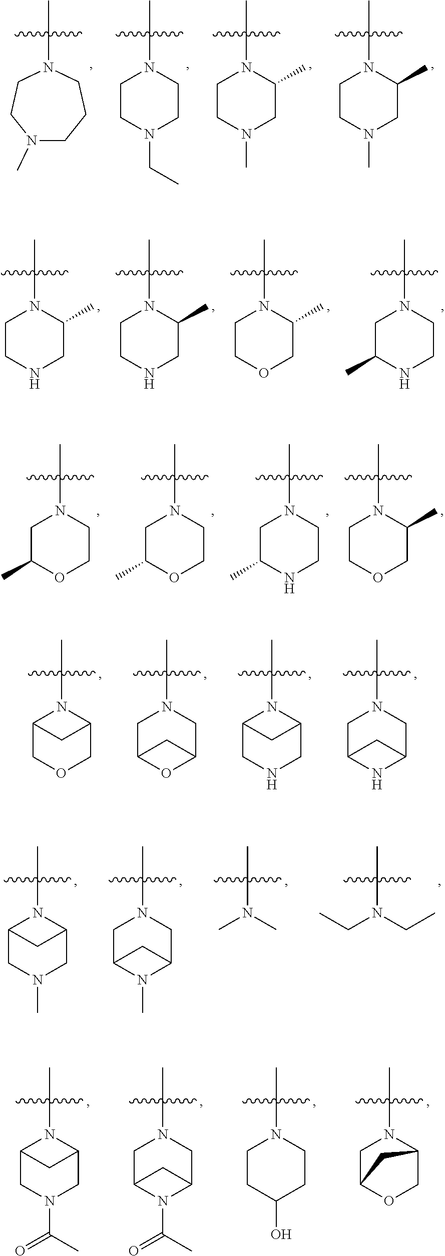

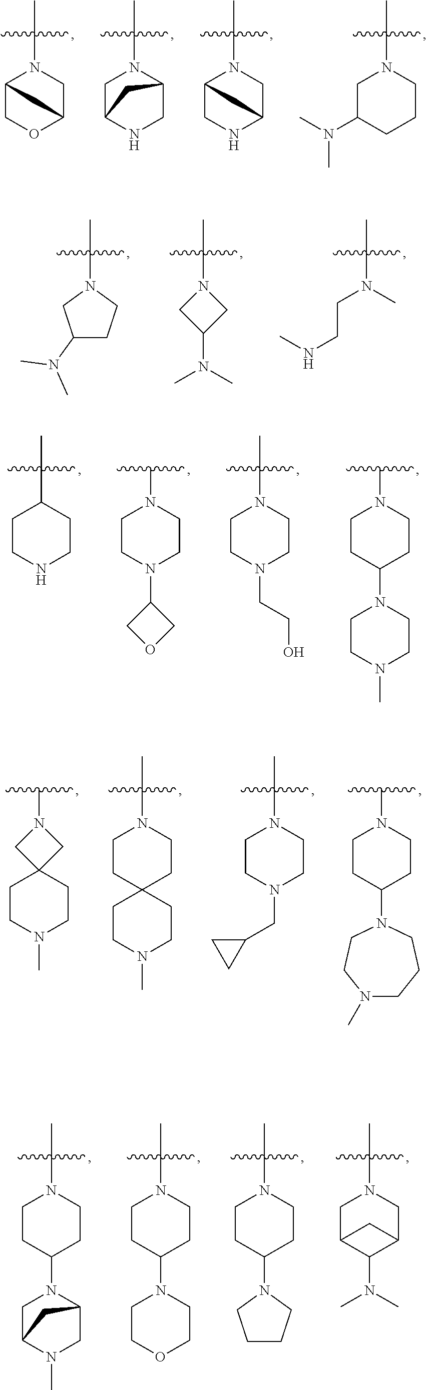

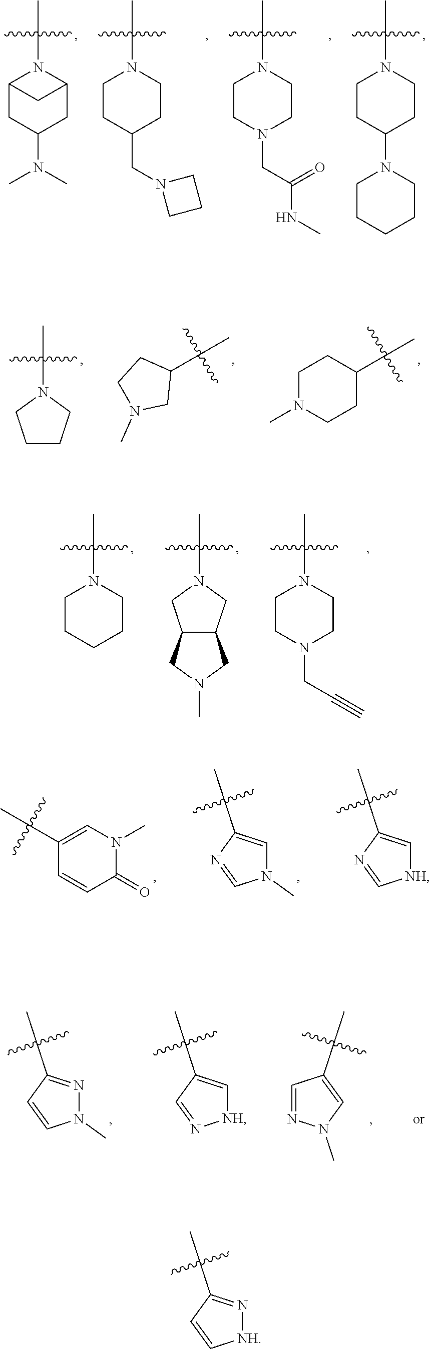

In another preferred example, R₄ is selected from the group consisting of: H,

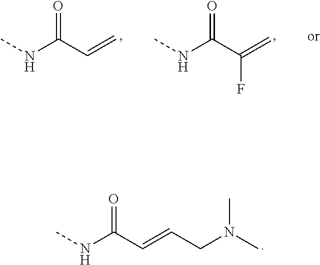

In another preferred example, it has structures shown in formulas (II) and (III):

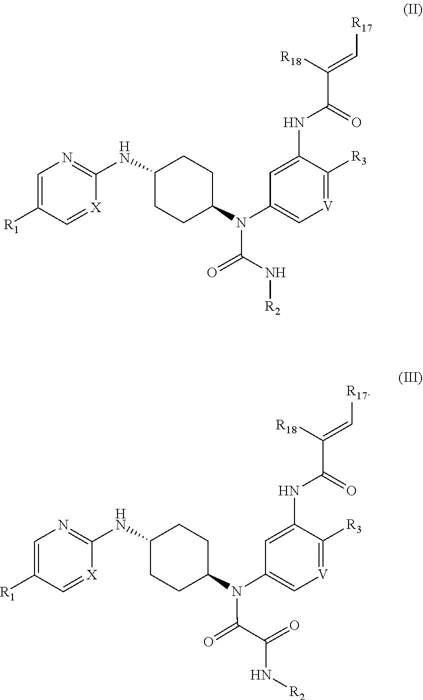

In another preferred example, the compound is selected from compounds in Examples 1 to 53.

A second aspect of the present invention provides use of the CDK12/13 covalent inhibitor according to the first aspect of the present invention or the pharmaceutical composition thereof in the preparation of a CDK12/13 inhibitor.

A third aspect of the present invention provides use of the CDK12/13 covalent inhibitor according to the first aspect of the present invention, or the pharmaceutical composition thereof, or the pharmaceutically acceptable salt thereof, or the stereoisomer thereof, or the prodrug molecule thereof in the preparation of a drug for preventing and/or treating a disease mediated by a CDK12/13 serine/threonine protein kinase.

In another preferred example, the disease mediated by the CDK12/13 serine/threonine protein kinase is selected from the group consisting of: prostate cancer, breast cancer, uterine cancer, ovarian cancer, non-small cell lung cancer, small cell lung cancer, Ewing sarcoma, lung adenocarcinoma, squamous cell lung carcinoma, pancreatic cancer, liver cancer, skin cancer, epithelial cell carcinoma, gastrointestinal stromal tumor, leukemia, histiocytic lymphoma, nasopharyngeal carcinoma, head and neck tumor, colon cancer, rectal cancer, or glioma.

A fourth aspect of the present invention provides a pharmaceutical composition for preventing and/or treating a tumor, comprising an active ingredient and a pharmaceutically acceptable adjuvant; where the active ingredient comprises the class of novel CDK12/13 covalent inhibitors according to the first aspect of the present invention, or the pharmaceutical composition thereof, or the pharmaceutically acceptable salt thereof, or the stereoisomer thereof, or the prodrug molecule thereof.

It should be understood that within the scope of the present invention, the above technical features and the technical features specifically described below (e.g., in the examples) in the present invention may be combined with each other, thus forming novel or preferred technical solutions, which are not repeated one by one here due to limited space.

### DETAILED DESCRIPTION

Some examples thereof provide use of a class of novel CDK12/13 covalent inhibitors or a pharmaceutical composition thereof in the preparation of a CDK12/13 inhibitor.

Another objective of the present invention is to provide use of the above CDK12/13 covalent inhibitor or the pharmaceutical composition thereof in the preparation of a drug for preventing and/or treating a disease mediated by CDK12/13 serine/threonine protein kinase.

In some examples thereof, the disease mediated by the CDK12/13 serine/threonine protein kinase is preferably: any one of prostate cancer, breast cancer, uterine cancer, ovarian cancer, non-small cell lung cancer, small cell lung cancer, Ewing sarcoma, lung adenocarcinoma, squamous cell lung carcinoma, pancreatic cancer, liver cancer, skin cancer, epithelial cell carcinoma, gastrointestinal stromal tumor, leukemia, histiocytic lymphoma, nasopharyngeal carcinoma, head and neck tumor, colon cancer, rectal cancer, and glioma.

Still another objective of the present invention is to provide a pharmaceutical composition for preventing and/or treating a tumor, where the pharmaceutical composition comprises an active ingredient and a pharmaceutically acceptable adjuvant, and the active ingredient comprises the trans-1,4-cyclohexanediamine compound, or the pharmaceutically acceptable salt thereof, or the stereoisomer thereof, or the prodrug molecule thereof.

### Terms

The experimental methods without clear indication of specific conditions in the following examples of the present invention are carried out usually based on general conditions, or based on conditions recommended by manufacturers. Various common chemical reagents used in the examples are all commercially available products.

Unless otherwise defined, all technical terms and scientific terms used in the present invention have the same meaning as commonly understood by those skilled in the technical field of the present invention. The terms used in the specification of the present invention are only for the purpose of describing specific examples, and are not used to limit the present invention.

The terms “comprising” and “having” and any variations thereof in the present invention are intended to encompass non-exclusive inclusions. For example, a process, method, apparatus, product, or device that includes a series of steps is not limited to the listed steps or modules, but optionally further includes unlisted steps, or optionally further includes other steps that are inherent to such a process, method, product or device.

The “plurality of” mentioned in the present invention refers to two or more than two. The term “and/or” describes an association relationship between associated objects, and means that there may be three relationships. For example, A and/or B may mean three situations: A exists alone, both A and B exist, or B exists alone. The character “/” generally means that there is an “or” relationship between associated objects therebefore and thereafter.

In the compound of the present invention, when any variable (e.g., R₁₀, or R₁₁) occurs more than once in any component, the definition of the variable at each occurrence is independent of the definition of the variable at each other occurrence. Likewise, a combination of substituents and variables is allowed, as long as such a combination makes a compound stable. A line started from a substituent into a ring system means that the indicated bond may be attached to any substitutable ring atom. If the ring system is polycyclic, it means that such a bond is only attached to any appropriate carbon atom on an adjacent ring. It should be understood that those of ordinary skills in the art may select a substituent and a substitution form for the compound of the present invention to provide a compound that is chemically stable and is readily synthesized from readily available starting materials in accordance with a technique in the art and a method set forth below. If a substituent itself is substituted with more than one group, it should be understood that these groups may be on the same carbon atom or on different carbon atoms, as long as the structure is stable.

As used herein, the term “alkyl” is intended to include a branched or straight saturated aliphatic hydrocarbon radical having a particular number of carbon atoms. For example, the definition of “C₁-C₈” in “C₁-C₈ alkyl” includes a group having 1, 2, 3, 4, 5, 6, 7, or 8 carbon atoms in a straight or branched arrangement. The term “cycloalkyl” refers to a monocyclic saturated aliphatic hydrocarbon radical having a particular number of carbon atoms. For example, the “cycloalkyl” includes cyclopropyl, methyl-cyclopropyl, 2,2-dimethyl-cyclobutyl, 2-ethyl-cyclopentyl, cyclohexyl, and the like.

As used herein, the term “alkoxy” denotes an alkyl-oxy group, where the alkyl is as defined above.

Just as understood by those skilled in the art, the “halogen” as used herein is intended to include chlorine, fluorine, bromine, and iodine.

The present invention not only includes a free form of the compound of formula (I), but also includes a pharmaceutically acceptable salt thereof, a stereoisomer thereof, and a prodrug molecule thereof. The term “free form” refers to a compound in a non-salt form. The included pharmaceutically acceptable salt not only includes example salts of the particular compounds described herein, but also includes typical pharmaceutically acceptable salts in free forms of all compounds of formula (I). The free forms of the particular salts of the compounds may be isolated using techniques known in the art. For example, the free form may be regenerated by treating the salt with an appropriate dilute aqueous base such as dilute aqueous NaOH, dilute aqueous potassium carbonate, dilute aqueous ammonia, and dilute aqueous sodium bicarbonate. The free forms differ somewhat from their respective salt forms in some physical properties such as solubility in a polar solvent, but for the purpose of the invention, such acidic and basic salts are otherwise pharmaceutically equivalent to their respective free forms.

### Compounds and Pharmaceutically Acceptable Salts

Disclosed herein are compounds of formula (I), including compounds of formulae (II) and (III), the structures of which are disclosed herein, as well pharmaceutically acceptable salts thereof.

The pharmaceutically acceptable salt of the present invention may be synthesized from the compound of the present invention comprising a basic or acidic moiety in accordance with a conventional chemical method. Generally, a salt of an alkali compound is prepared by ion exchange chromatography or by reacting a free base with a stoichiometric amount or excess amount of an inorganic or organic acid in a desired salt form in a suitable solvent or combination of a plurality of solvents. Similarly, a salt of an acidic compound is formed by a reaction with a suitable inorganic or organic base.

Accordingly, the pharmaceutically acceptable salt of the compound of the present invention includes a conventional non-toxic salt of the compound of the present invention formed by reacting an alkali compound of the present invention with an inorganic or organic acid. For example, the conventional non-toxic salt not only includes a salt prepared from an inorganic acid, such as hydrochloric acid, hydrobromic acid, sulfuric acid, sulfamic acid, phosphoric acid, or nitric acid, but also includes a salt prepared from an organic acid, such as acetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, p-aminobenzenesulfonic acid, 2-acetoxymonobenzoic acid, fumaric acid, benzenesulfonic acid, toluenesulfonic acid, methanesulfonic acid, ethanedisulfonic acid, oxalic acid, isethionic acid, or trifluoroacetic acid.

If the compound of the present invention is acidic, an appropriate “pharmaceutically acceptable salt” refers to a salt prepared from a pharmaceutically acceptable non-toxic base including an inorganic base and an organic base. A salt derived from an inorganic base includes an aluminum salt, an ammonium salt, a calcium salt, a copper salt, a ferric salt, a ferrous salt, a lithium salt, a magnesium salt, a manganese salt, a manganous salt, a potassium salt, a sodium salt, a zinc salt, and the like, and is particularly preferably an ammonium salt, a calcium salt, a magnesium salt, a potassium salt, and a sodium salt. A salt derived from a pharmaceutically acceptable organic non-toxic base includes a salt of a primary amine, a secondary amine, and a tertiary amine, and a substituted amine includes a naturally occurring substituted amine, a cyclic amine, and an basic ion exchange resin, such as arginine, betaine, caffeine, choline, N,N′-dibenzylethylenediamine, diethylamine, 2-diethylaminoethanol, 2-dimethylaminoethanol, aminoethanol, ethanolamine, ethyldiamine, N-ethylmorpholine, N-ethylpiperidine, glucosamine, glucosamine, histidine, hydroxocobalamin, isopropylamine, lysine, methylglucamine, morpholine, piperazine, piperidine, piperidine, polyamine resin, procaine, purine, theobromine, triethylamine, trimethylamine, tripropylamine, and tromethamine.

Berg et al, “Pharmaceutical Salts” J. Pharm. Sci. '1977: 66: 1-19 describes in more detail the preparation of the pharmaceutically acceptable salt described above and other typical pharmaceutically acceptable salts.

The disclosed compounds include at least one asymmetric or chiral center, and accordingly exist as stereoisomers. Each chiral center is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem. 1976, 45: 13-30. Various stereoisomers and mixtures thereof are specifically included within the scope of this disclosure. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by: (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM202JE, England; (2) direct separation of the mixture of optical enantiomers on chiral chromatographic columns; or (3) fractional recrystallization methods.

The disclosed compounds may exist in different tautomeric forms, and all such forms are included within the scope of the disclosure.

The compounds of the present disclosure may also be in prodrug form. As used herein, the term “prodrug” refers to a compound which, when metabolized (e.g., in vivo or in vitro), yields an active compound. In some embodiments, the prodrug may be inactive, or possess less activity that the free drug, but may provide advantageous handling, administration, or metabolic properties. Exemplary prodrug moieties of the present invention may be linked to the free drug through the hydroxyl, amino, phosphate, or phosphorothioate backbone of the nucleotide, and may comprise an ester, a carbamate, a carbonyl, a thioester, amide, isocyanate, urea, thiourea, or other physiologically acceptable metabolically labile moiety. In some embodiments, a prodrug is activated through enzymatic hydrolysis.

The present disclosure also includes isotopically-labeled compounds, which are identical to those recited in formula (I), formula (II), or formula (III), but for the fact that one or more atoms are replaced by an atom having an atomic mass or mass number different from the atomic mass or mass number usually found in nature. Examples of isotopes suitable for inclusion in the compounds of the disclosure are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ³¹P, ³⁵S, ¹⁸F, and ³⁶Cl, respectively. Substitution with heavier isotopes such as deuterium, i.e. ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors.

Suitable positron-emitting isotopes that can be incorporated in compounds of formula (I), (II), or (III) are ¹¹C, ¹³N, ¹⁵O, and ¹⁸F. Isotopically-labeled compounds of formula (I), (II), or (III) can generally be prepared by conventional techniques known to those skilled in the art or by processes analogous to those described herein using an appropriate isotopically-labeled reagent in place of a non-isotopically-labeled reagent.

Compounds disclosed herein can exist in solvated as well as unsolvated forms with pharmaceutically acceptable solvents such as water, ethanol, and the like, and it is intended that the disclosure encompass both solvated and unsolvated forms. In one embodiment, the compound is amorphous. In one embodiment, the compound is a single polymorph. In another embodiment, the compound is a mixture of polymorphs. In another embodiment, the compound is in a crystalline form.

### Pharmaceutical Composition and Administration Method

Since the compounds of the present disclosure are CDK12/13 inhibitors, the compounds and pharmaceutically acceptable salts thereof, as well as other compound forms disclosed herein, can be included in a pharmaceutical composition that may be used to treat, prevent, and alleviate a disease associated with the activity of CDK12/13.

The pharmaceutical composition of the present disclosure comprises the compound of the present disclosure or a pharmacologically acceptable salt thereof and a pharmacologically acceptable excipient or carrier within an effective amount, e.g., a safe and effective amount. The “effective amount” refers to an amount sufficient to elicit a desired biological response (e.g., treating a condition). The “safe and effective amount” means that: the amount of the compound is sufficient to significantly ameliorate the condition without causing severe side effects. Typically, the pharmaceutical composition comprises from 1 to 3000 (active dose ranging from 3 to 30 mg/kg) mg of the compound/dose of the present disclosure, and more preferably comprises from 10 to 2000 mg of the compound/dose of the present disclosure. Preferably, the “one dose” is a capsule or tablet.

The “pharmaceutically acceptable carrier” refers to: one or more compatible solids or liquid fillers or gel substances which are suitable for human use, and must be of sufficient purity and sufficiently low toxicity. The “compatibility” herein means that the components of the composition can be admixed with the compound of the present disclosure and with each other without significantly reducing the efficacy of the compound. Examples of pharmaceutically acceptable carrier moieties include cellulose and derivatives thereof (such as sodium carboxymethylcellulose, sodium ethylcellulose, and cellulose acetate), gelatin, talc, solid lubricants (such as stearic acid and magnesium stearate), calcium sulfate, vegetable oils (such as soybean oil, sesame oil, peanut oil, and olive oil), polyol (such as propylene glycol, glycerol, mannitol, and sorbitol), emulsifiers (such as Tween®), wetting agents (such as sodium lauryl sulfate), colorants, flavoring agents, stabilizers, antioxidants, preservatives, and pyrogen-free water.

The administration mode of the compound or the pharmaceutical composition of the present disclosure is not particularly limited, and the representative administration mode includes (but is not limited to): oral, intratumoral, rectal, parenteral (intravenous, intramuscular or subcutaneous), and topical administration.

Solid dosage forms for oral administration include capsules, tablets, pills, powders, and granules. In these solid dosage forms, an active compound is mixed with at least one conventional inert excipient (or carrier), such as sodium citrate or dicalcium phosphate, or with the following ingredients: (a) a filler or a bulking agent, such as starch, lactose, sucrose, glucose, mannitol, and silicic acid; (b) a binder, such as hydroxymethylcellulose, alginate, gelatin, polyvinylpyrrolidone, sucrose, and acabic gum; (c) a humectant, such as glycerin; (d) a disintegrant, such as agar, calcium carbonate, potato starch or tapioca starch, alginic acid, some complex silicates, and sodium carbonate; (e) a retarding solvent, such as paraffin; (f) an absorption accelerator, such as a quaternary amine compound; (g) a wetting agent, such as cetyl alcohol and glyceryl monostearate; (h) an adsorbent, such as kaolin; and (i) a lubricant, such as talc, calcium stearate, magnesium stearate, solid polyethylene glycol, sodium lauryl sulfate, or a mixture thereof. In a capsule, a tablet, and a pill, the dosage form may further include a buffering agent.

The solid dosage forms, such as tablets, dragees, capsules, pills, and granules, may be prepared from coatings and shell materials, such as an enteric coating and other materials well known in the art. They may comprise an opacifying agent, and an active compound or a compound in such a composition may be released in a portion of the digestive tract in a delayed manner. Examples of available embedding components are polymeric substances and waxes. When necessary, the active compound may further form a microencapsule form with one or more of the above excipients.

Liquid dosage forms for oral administration include a pharmaceutically acceptable emulsion, solution, suspension, syrup, or elixir. In addition to the active compound, the liquid dosage forms may comprise an inert diluent commonly used in the art, such as water or other solvents, a solubilizing agent and an emulsifier, such as ethanol, isopropanol, ethyl carbonate, ethyl acetate, propanediol, 1,3-butanediol, dimethylformamide, and an oil, in particular, a cottonseed oil, a peanut oil, a maize germ oil, an olive oil, a castor oil, and a sesame oil, or a mixture thereof.

Besides these inert diluents, the composition may further comprise an auxiliary agent, such as a wetting agent, an emulsifier and a suspending agent, a sweetening agent, a corrigent, and a perfume.

Besides the active compound, the suspension may comprise a suspending agent, such as ethoxylated isooctadecanol, polyoxyethylene sorbitol and sorbitan ester, microcrystalline cellulose, aluminum methoxide, and agar, or a mixture thereof.

A composition for parenteral injection may comprise a physiologically acceptable sterile aqueous or anhydrous solution, dispersion, suspension or emulsion, and a sterile powder for redissolution into a sterile injectable solution or dispersion. An appropriate aqueous and non-aqueous carrier, diluent, solvent, or excipient comprises water, ethanol, polyol, and an appropriate mixture thereof.

Dosage forms for topical administration of the compound of the present disclosure include ointments, powders, patches, sprays, and inhalants. An active ingredient is mixed under sterile conditions with a physiologically acceptable carrier and any preservative, buffering agent, or a propellant (if necessary).

The compound of the present disclosure may be administered alone or may be administered in combination with other pharmaceutically acceptable compounds.

When the pharmaceutical composition is used, a safe and effective amount of the compound of the present disclosure is suitable for a mammal (such as human) in need of treatment, where the dose is a pharmaceutically considered effective dose when administered, and the daily administration dose for a person with a body weight of 60 kg is generally from 1 to 2000 mg, and preferably from 6 to 600 mg. Of course, a specific dose should also be determined by considering factors, such as the route of administration and the health condition of a patient, which are all within the scope of skills of skilled physicians.

### Uses and Methods of Treatment

As described above, compounds of the present disclosure are CDK12/13 inhibitors, and accordingly the compounds, or compositions comprising the compounds, can be used to treat, prevent, and alleviate a disease associated with the activity or abnormal expression of CDK12/13. In some embodiments, disclosed herein is the use of compound disclosed herein in the preparation of a drug for preventing and/or treating a disease mediated by a CDK12/13 serine/threonine protein kinase. In some embodiments, disclosed herein is a compound disclosed herein for use in preventing and/or treating a disease mediated by a CDK12/13 serine/threonine protein kinase. In some embodiments, disclosed herein is a method of treating a disease mediated by a CDK12/13 serine/threonine protein kinase in a subject in need thereof, comprising administering to the subject an effective amount of a compound disclosed herein. In some embodiments, the disease mediated by the CDK12/13 serine/threonine protein kinase comprises: prostate cancer, breast cancer, uterine cancer, ovarian cancer, non-small cell lung cancer, small cell lung cancer, Ewing sarcoma, lung adenocarcinoma, squamous cell lung carcinoma, pancreatic cancer, liver cancer, skin cancer, epithelial cell carcinoma, gastrointestinal stromal tumor, leukemia, histiocytic lymphoma, nasopharyngeal carcinoma, head and neck tumor, colon cancer, rectal cancer, and glioma.

When used in the uses and methods disclosed herein, the disclosed compounds and compositions may be used in combination with other known therapies. Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A compound or composition disclosed herein and the at least one additional therapeutic agent can be administered simultaneously, in the same or in separate compositions, or sequentially. For sequential administration, the compound described herein can be administered first, and the additional agent can be administered subsequently, or the order of administration can be reversed.

In some embodiments, a compound described herein is administered in combination with other therapeutic treatment modalities, including surgery, radiation, transplantation (e.g., stem cell transplantation, bone marrow transplantation), chemotherapy, immunotherapy, cryotherapy, and/or thermotherapy. Such combination therapies may allow for lower dosages of the administered agent and/or other agent, thus avoiding possible toxicities or complications associated with the various therapies.

In some embodiments, the compound described herein is administered with at least one additional therapeutic agent, such as a chemotherapeutic agent. In certain embodiments, the compound described herein is administered in combination with one or more additional chemotherapeutic agents. The chemotherapeutic agent may be a chemotherapeutic agent identified on the “A to Z List of Cancer Drugs” published by the National Cancer Institute.

Compared with the Prior Art, the Present Invention has the Following Beneficial Effects:

The novel CDK12/13 covalent inhibitor provided in the present invention or a pharmaceutical composition thereof can effectively inhibit a CDK12 protein kinase, can be used in the preparation of a drug for preventing or treating a disease mediated by the CDK12 protein kinase, such as prostate cancer, breast cancer, uterine cancer, ovarian cancer, non-small cell lung cancer, small cell lung cancer, Ewing sarcoma, lung adenocarcinoma, squamous cell lung carcinoma, pancreatic cancer, liver cancer, skin cancer, epithelial cell carcinoma, gastrointestinal stromal tumor, leukemia, histiocytic lymphoma, nasopharyngeal carcinoma, head and neck tumor, colon cancer, rectal cancer, and glioma, and has strong inhibitory activity, kinase selectivity, and good pharmacokinetic characteristics.

The present invention will be further described below with reference to specific examples. It should be understood that these examples are provided only to illustrate the present invention, rather than limiting the scope of the present invention. The experimental methods without clear indication of specific conditions in the following examples are carried out usually based on general conditions, or based on conditions recommended by manufacturers. Unless otherwise indicated, all percentages and parts are by weight.

### Example 1: N-(5-(3-benzyl-1-((1r,4r)-4-((5-cyanopyridin-2-yl)amino)cyclohexyl)ureido)-2-(4-(dimethylamino)piperidin-1-yl)phenyl)acrylamide (YJZ5118)

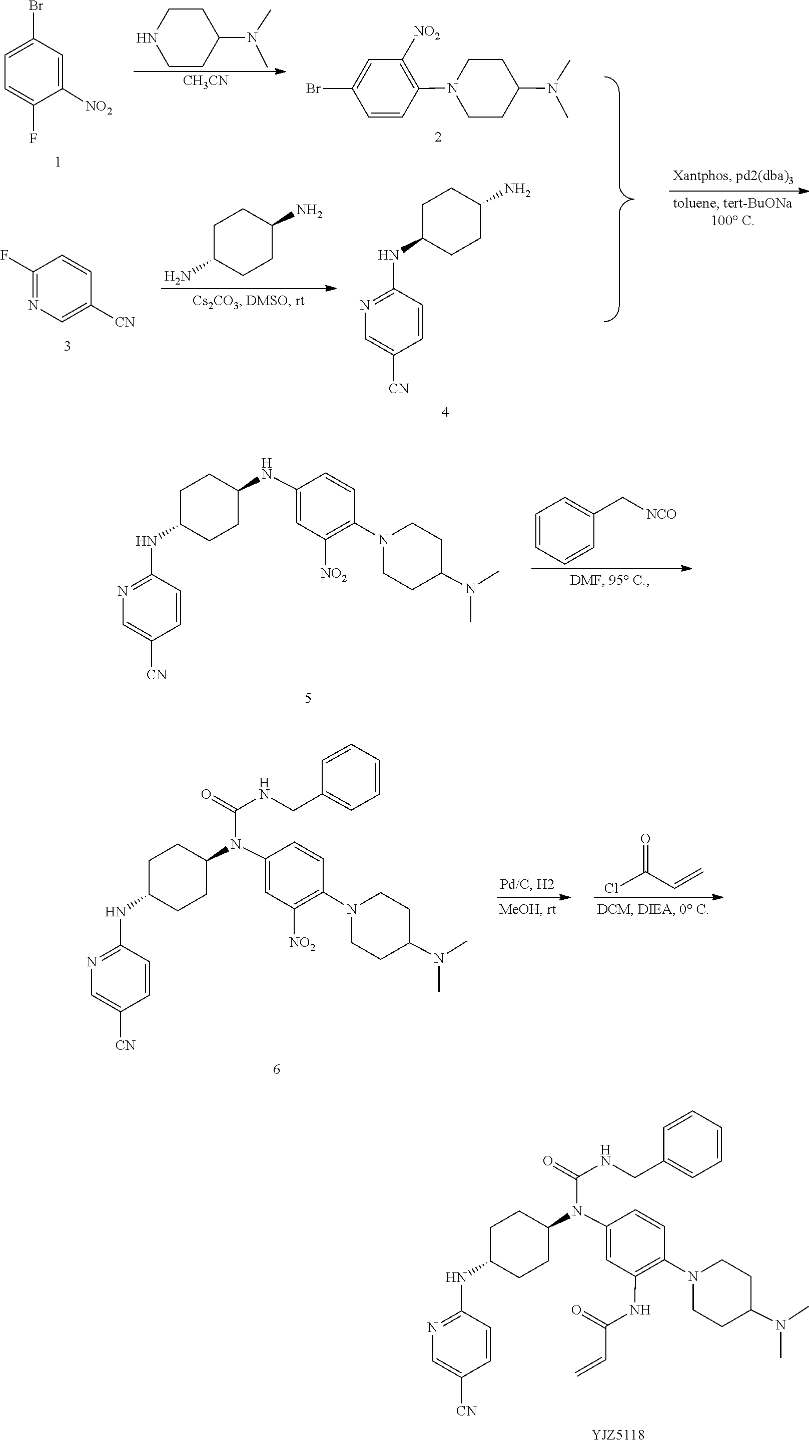

### Step 1: 1-(4-bromo-2-nitrophenyl)-N, N-dimethylpiperidin-4-amine (2)

4-bromo-1-fluoro-2-nitrobenzene (1.72 g, 7.8 mmol) was dissolved in 50 ml of acetonitrile solvent, N,N-dimethylpiperidin-4-amine (1 g, 7.8 mmol) was slowly added, and the mixture was kept at room temperature for reaction for 3 h. When TLC detection showed that the raw material 1 was completely consumed, the solvent was spin-dried under reduced pressure, to provide an orange-yellow oily liquid, which was directly used in the next step reaction without any purification.

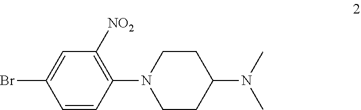 [SMILES: CN(C)C1CCN(c2ccc(Br)cc2[N+](=O)[O-])CC1]

### Step 2: 6-6-(((((1r,4r)-4-aminocyclohexyl)amino)nicotinonitrile (4)

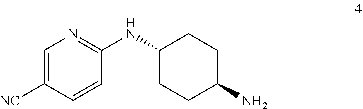 [SMILES: N#Cc1ccc(N[C@H]2CC[C@H](N)CC2)nc1]

¹H NMR (400 MHz, Chloroform-d) δ 8.35 (d, J=2.2 Hz, 1H), 7.55 (dd, J=8.8, 2.3 Hz, 1H), 6.35 (d, J=8.9 Hz, 1H), 5.05-4.84 (m, 1H), 3.64 (s, 1H), 2.73 (dq, J=7.0, 4.2, 3.6 Hz, 1H), 2.12 (dq, J=7.6, 3.5, 2.9 Hz, 2H), 1.94 (tt, J=5.6, 2.4 Hz, 2H), 1.34 (d, J=6.3 Hz, 2H), 1.32-1.21 (m, 4H).

Trans-1,4-cyclohexanediamine (10 g, 87.6 mol) and CsCO3 (34 g, 105.1 mol) were dissolved in 100 ml of DMSO, and 6-fluoronicotine (3 g, 25 mol) was slowly added in five portions at 10 min intervals. The mixture was stirred at room temperature for 1 h, and heated to 60° C. for reaction for an additional 1 h. After TLC detection showed that 7 was completely consumed, DMSO was spin-dried under vacuum, and the concentrated product was dissolved in DCM and an appropriate amount of methanol. The solution was filtered through diatomite, and the filter cake was washed with dichloro several times. Then, the filtrate was collected, concentrated, and subjected to silica gel column chromatography to provide a white solid product (3.4 g, yield: 63%).

### Step 3: 6-((((1r,4r)-4-((4-(4-(dimethylamino)piperidin-1-yl)-3-nitrophenyl)amino)cyclohexyl)amino)nicotinonitrile (5)

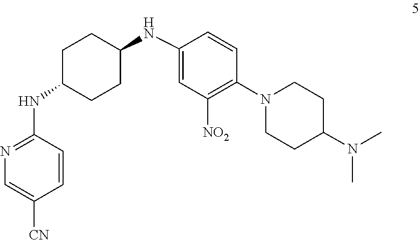 [SMILES: CN(C)C1CCN(c2ccc(N[C@H]3CC[C@H](Nc4ccc(C#N)cn4)CC3)cc2[N+](=O)[O-])CC1]

Compound 2 (2.5 g, 7.6 mol), compound 4 (2 g, 9.1 mol), Xantphos (439.3 mg, 0.76 mol), Pd₂(dba)₃ (348 mg, 0.38 mol), and tert-BuONa (1.46 g, 15.2 mol) were dissolved in 100 ml of toluene. The solution was replaced under the protection of argon three times, stirred at 95° C. for reaction overnight, cooled to room temperature, and then filtered through diatomite. The solvent was spin-dried, and separated by column chromatography to provide a deep red solid (1.9 g, yield: 54%).

¹H NMR (400 MHz, DMSO-d₆) δ 8.38 (d, J=2.3 Hz, 1H), 7.64 (dd, J=8.8, 2.4 Hz, 1H), 7.54 (d, J=7.5 Hz, 1H), 7.17 (d, J=8.9 Hz, 1H), 6.87 (d, J=2.7 Hz, 1H), 6.80 (dd, J=8.9, 2.8 Hz, 1H), 6.53 (d, J=8.9 Hz, 1H), 5.83 (d, J=8.0 Hz, 1H), 3.77 (s, 1H), 3.28-3.13 (m, 1H), 2.97 (d, J=10.8 Hz, 2H), 2.64 (td, J=11.7, 2.3 Hz, 2H), 2.17 (s, 6H), 2.11 (dq, J=6.1, 3.5 Hz, 1H), 1.98 (q, J=9.3, 8.1 Hz, 4H), 1.79-1.70 (m, 2H), 1.47-1.30 (m, 4H), 1.29-1.18 (m, 2H).

### Step 4: 3-benzyl-1-((1r,4r)-4-((5-cyanopyridin-2-yl)amino)cyclohexyl)-1-(4-(4-(dimethylamino)piperidin-1-yl)-3-nitrophenyl)urea (6)

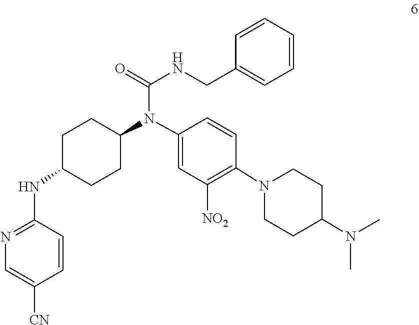 [SMILES: CN(C)C1CCN(c2ccc(N(C(=O)NCc3ccccc3)[C@H]3CC[C@H](Nc4ccc(C#N)cn4)CC3)cc2[N+](=O)[O-])CC1]

Compound 5 (1.9 g, 4.1 mol) was dissolved in 10 ml of DMF. Benzyl isocyanate (1.64 g, 12.3 mol) and DIEA (1.59 g, 12.3 mol) were added. The mixture was kept at 95° C. for reaction for 5 h, and, after the solvent was spin-dried, was subjected to column chromatography to provide a yellow solid (2 g, yield: 83%).

¹H NMR (400 MHz, DMSO-d₆) δ 8.30 (d, J=2.3 Hz, 1H), 7.61 (dd, J=8.9, 2.3 Hz, 1H), 7.58 (d, J=2.4 Hz, 1H), 7.49 (d, J=7.8 Hz, 1H), 7.35 (dd, J=8.8, 2.5 Hz, 1H), 7.32-7.25 (m, 3H), 7.18 (td, J=5.5, 3.0 Hz, 3H), 6.48 (d, J=9.0 Hz, 1H), 6.24 (t, J=6.1 Hz, 1H), 4.33-4.21 (m, 1H), 4.16 (d, J=6.1 Hz, 2H), 3.51 (s, 1H), 3.28 (d, J=12.4 Hz, 2H), 2.86 (td, J=12.2, 2.3 Hz, 2H), 2.24 (s, 1H), 2.21 (s, 6H), 1.92 (d, J=12.0 Hz, 2H), 1.82 (t, J=12.7 Hz, 4H), 1.49 (qd, J=11.9, 3.8 Hz, 2H), 1.31 (q, J=14.8, 13.7 Hz, 2H), 1.18-1.04 (m, 2H).

### Step 5: N-(5-(3-benzyl-1-((1r,4r)-4-((5-cyanopyridin-2-yl)amino)cyclohexyl)ureido)-2-(4-(dimethylamino)piperidin-1-yl)phenyl)acrylamide (YJZ5118)

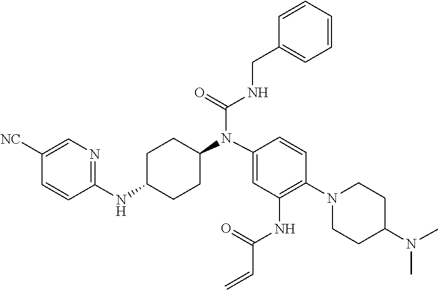 [SMILES: C=CC(=O)Nc1cc(N(C(=O)NCc2ccccc2)[C@H]2CC[C@H](Nc3ccc(C#N)cn3)CC2)ccc1N1CCC(N(C)C)CC1]

Compound 6 (2 g, 3.35 mol) and 10% Pd/C (200 mg) were dissolved in 70 ml of methanol. The solution was kept under a hydrogen atmosphere at room temperature for reaction for 3 h. When the reaction was complete, the mixture was suction-filtered through diatomite, and the solvent was spin-dried, to provide a preliminary product, which was directly used in the next step without purification. The preliminary product in this step was dissolved in 50 ml of anhydrous DCM, and DIEA (518.8 mg, 4 mol) was added. After the mixture was stirred at 0° C. for 15 min, acryloyl chloride (364 mg, 4 mol) was slowly added. After reaction for 10 min, the mixture was warmed to room temperature for reaction for an additional 15 min. After TLC monitoring showed that the reaction was complete, DCM was spin-dried; the mixture was extracted with ethyl acetate and saturated sodium bicarbonate; and the organic phase was collected, concentrated, and subjected to column chromatography to provide 410 mg (total yield of two steps: 20%).

¹H NMR (400 MHz, DMSO-d₆) δ 9.01 (s, 1H), 8.31 (d, J=2.4 Hz, 1H), 7.92 (s, 1H), 7.60 (dd, J=9.0, 2.4 Hz, 1H), 7.49 (d, J=7.5 Hz, 1H), 7.30-7.23 (m, 2H), 7.22-7.13 (m, 4H), 6.90 (dd, J=8.3, 2.5 Hz, 1H), 6.73 (dd, J=16.9, 10.2 Hz, 1H), 6.47 (d, J=8.9 Hz, 1H), 6.28 (dd, J=17.0, 2.0 Hz, 1H), 5.84-5.72 (m, 2H), 4.32-4.22 (m, 1H), 4.17 (d, J=6.0 Hz, 2H), 3.50 (s, 1H), 3.07 (d, J=11.4 Hz, 2H), 2.65 (t, J=11.2 Hz, 2H), 2.23 (s, 6H), 2.17 (d, J=11.1 Hz, 1H), 1.92 (d, J=11.9 Hz, 2H), 1.85 (d, J=11.4 Hz, 2H), 1.78 (d, J=12.9 Hz, 2H), 1.74-1.68 (m, 2H), 1.32 (q, J=12.2 Hz, 2H), 1.22-1.09 (m, 2H).

HRMS (ESI) calcd for C₃₆H₄₄N₈O₂ [M+H]⁺, 621.3660; found, 621.3666.

### Example 2: N-(2-(4-acetylpiperazin-1-yl)-5-(3-benzyl-1-((1r,4r)-4-((5-cyanopyridin-2-yl)amino)cyclohexyl)ureido)phenyl)acrylamide (YJZ5111)

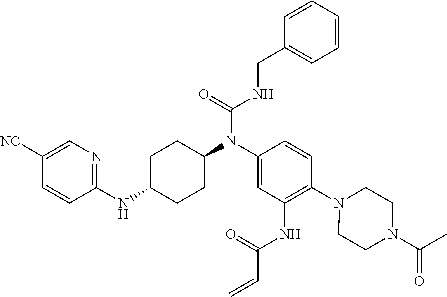 [SMILES: C=CC(=O)Nc1cc(N(C(=O)NCc2ccccc2)[C@H]2CC[C@H](Nc3ccc(C#N)cn3)CC2)ccc1N1CCN(C(C)=O)CC1]

The synthetic method was similar to the method as shown in Example 1.

¹H NMR (400 MHz, DMSO-d₆) δ 9.15 (s, 1H), 8.31 (dd, J=2.4, 0.7 Hz, 1H), 8.02-7.95 (m, 1H), 7.61 (dd, J=8.8, 2.4 Hz, 1H), 7.50 (d, J=7.6 Hz, 1H), 7.30-7.24 (m, 2H), 7.22 (d, J=8.4 Hz, 1H), 7.18 (ddd, J=7.0, 4.5, 2.7 Hz, 3H), 6.92 (dd, J=8.4, 2.5 Hz, 1H), 6.75 (dd, J=16.9, 10.2 Hz, 1H), 6.48 (d, J=8.9 Hz, 1H), 6.30 (dd, J=17.0, 1.9 Hz, 1H), 5.85-5.78 (m, 2H), 4.26 (d, J=11.9 Hz, 1H), 4.17 (d, J=6.0 Hz, 2H), 3.72-3.63 (m, 4H), 3.51 (s, 1H), 2.86 (t, J=5.0 Hz, 2H), 2.81 (t, J=5.1 Hz, 2H), 2.06 (s, 3H), 1.93 (d, J=9.7 Hz, 2H), 1.79 (d, J=11.4 Hz, 2H), 1.32 (q, J=12.4, 12.0 Hz, 2H), 1.18 (q, J=12.4, 12.0 Hz, 2H).

HRMS (ESI) calcd for C₃₅H₄₀N₈O₃[M+H]⁺, 621.3296; found, 621.3295.

### Example 3: N-(5-(3-benzyl-1-((1r,4r)-4-((5-cyanopyridin-2-yl)amino)cyclohexyl)ureido)-2-(4-methylpiperazin-1-yl)phenyl)acrylamide (YJZ5114)

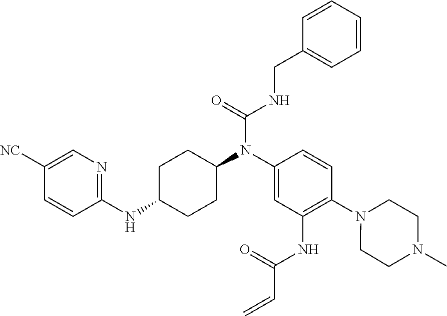 [SMILES: C=CC(=O)Nc1cc(N(C(=O)NCc2ccccc2)[C@H]2CC[C@H](Nc3ccc(C#N)cn3)CC2)ccc1N1CCN(C)CC1]

The synthetic method was similar to the method as shown in Example 1.

¹H NMR (400 MHz, DMSO-d₆) δ 9.08 (s, 1H), 8.31 (d, J=2.3 Hz, 1H), 8.05 (s, 1H), 7.61 (dd, J=8.9, 2.4 Hz, 1H), 7.52 (d, J=7.6 Hz, 1H), 7.31-7.24 (m, 3H), 7.18 (dt, J=6.4, 1.6 Hz, 3H), 6.95 (dd, J=8.4, 2.4 Hz, 1H), 6.75 (dd, J=16.9, 10.2 Hz, 1H), 6.47 (d, J=8.9 Hz, 1H), 6.32 (dd, J=17.0, 1.9 Hz, 1H), 5.89-5.79 (m, 2H), 4.34-4.22 (m, 1H), 4.18 (d, J=6.0 Hz, 2H), 3.51 (s, 5H), 3.18 (s, 2H) 3.07 (d, J=19.4 Hz, 2H), 2.88 (s, 3H), 1.92 (d, J=11.8 Hz, 2H), 1.80 (d, J=11.5 Hz, 2H), 1.39-1.27 (m, 2H), 1.21-1.09 (m, 2H).

HRMS (ESI) calcd for C₃₄H₄₀N₈O₂[M+H]⁺, 593.3347; found, 593.3365.

### Example 4: N-(5-(3-benzyl-1-((1r,4r)-4-((5-cyanopyridin-2-yl)amino)cyclohexyl)ureido)-2-morpholinophenyl)acrylamide (YJZ5127)

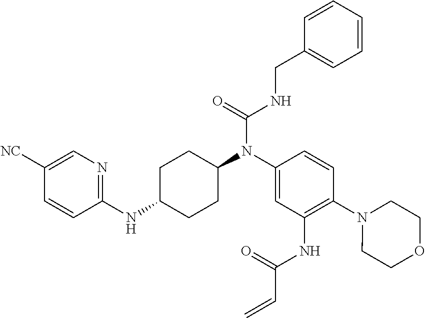 [SMILES: C=CC(=O)Nc1cc(N(C(=O)NCc2ccccc2)[C@H]2CC[C@H](Nc3ccc(C#N)cn3)CC2)ccc1N1CCOCC1]

The synthetic method was similar to the method as shown in Example 1.

¹H NMR (400 MHz, DMSO-d₆) δ 9.13 (s, 1H), 8.31 (dd, J=2.4, 0.7 Hz, 1H), 7.95 (s, 1H), 7.61 (dd, J=8.9, 2.4 Hz, 1H), 7.50 (d, J=7.6 Hz, 1H), 7.30-7.22 (m, 3H), 7.22-7.15 (m, 3H), 6.94 (dd, J=8.4, 2.5 Hz, 1H), 6.73 (dd, J=16.9, 10.2 Hz, 1H), 6.48 (d, J=8.9 Hz, 1H), 6.28 (dd, J=16.9, 1.9 Hz, 1H), 5.80 (dt, J=10.2, 3.3 Hz, 2H), 4.35-4.22 (m, 1H), 4.17 (d, J=6.0 Hz, 2H), 3.83 (t, J=4.5 Hz, 4H), 3.50 (s, H), 2.86 (t, J=4.6 Hz, 4H), 1.93 (d, J=11.9 Hz, 2H), 1.79 (d, J=11.6 Hz, 2H), 1.32 (q, J=12.2 Hz, 2H), 1.18 (q, J=12.2 Hz, 2H).

HRMS (ESI) calcd for C₃₃H₃₇N₇O₃[M+H]⁺, 580.3031; found, 5890.3024.

### Example 5: N-(5-(3-benzyl-1-((1r,4r)-4-((5-cyanopyridin-2-yl)amino)cyclohexyl)ureido)-2-((2-(dimethylamino)ethyl)(methyl)amino)phenyl)acrylamide (YJZ5125)

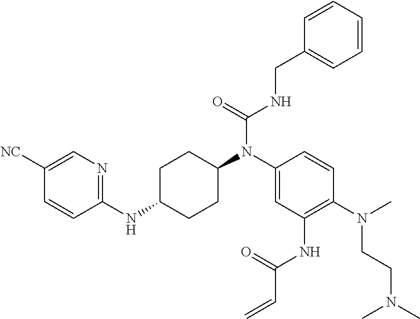 [SMILES: C=CC(=O)Nc1cc(N(C(=O)NCc2ccccc2)[C@H]2CC[C@H](Nc3ccc(C#N)cn3)CC2)ccc1N(C)CCN(C)C]

The synthetic method was similar to the method as shown in Example 1.

¹H NMR (400 MHz, DMSO-d₆) δ 10.22 (s, 1H), 8.31 (d, J=2.3 Hz, 1H), 8.24 (d, J=2.5 Hz, 1H), 7.61 (dd, J=8.9, 2.4 Hz, 1H), 7.50 (d, J=7.6 Hz, 1H), 7.33 (d, J=8.4 Hz, 1H), 7.30-7.24 (m, 2H), 7.21-7.14 (m, 3H), 6.89 (dd, J=8.4, 2.5 Hz, 1H), 6.48 (d, J=8.7 Hz, 1H), 6.45-6.39 (m, 1H), 6.30 (dd, J=17.0, 2.2 Hz, 1H), 5.82 (dd, J=9.9, 2.2 Hz, 1H), 5.76 (t, J=6.0 Hz, 1H), 4.33-4.23 (m, 1H), 4.18 (d, J=6.0 Hz, 2H), 3.52 (s, H), 2.83 (t, J=5.7 Hz, 2H), 2.72 (s, 3H), 2.40 (t, J=5.6 Hz, 2H), 2.23 (s, 6H), 1.93 (d, J=12.1 Hz, 2H), 1.79 (d, J=11.9 Hz, 2H), 1.32 (q, J=12.3 Hz, 2H), 1.18 (dd, J=28.1, 15.7 Hz, 2H).

HRMS (ESI) calcd for C₃₄H₄₂N₈O₂ [M+H]⁺, 595.3503; found, 595.3508.

### Example 6: N-(5-(3-benzyl-1-((1r,4R)-4-((5-cyanopyridin-2-yl)amino)cyclohexyl)ureido)-2-((1R,4R)-5-methyl-2,5-diazabicyclo[2.2.1]heptan-2-yl)phenyl)acrylamide (YJZ6096)

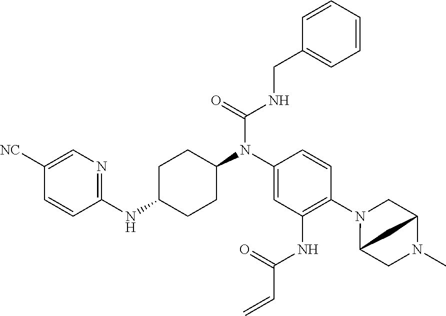 [SMILES: C=CC(=O)Nc1cc(N(C(=O)NCc2ccccc2)[C@H]2CC[C@H](Nc3ccc(C#N)cn3)CC2)ccc1N1C[C@H]2C[C@@H]1CN2C]

The synthetic method was similar to the method as shown in Example 1.

¹H NMR (400 MHz, DMSO-d₆) δ 9.30 (s, 1H), 8.33 (d, J=2.4 Hz, 1H), 7.61 (dd, J=8.8, 2.4 Hz, 1H), 7.49 (d, J=7.6 Hz, 1H), 7.30-7.23 (m, 2H), 7.17 (dt, J=9.0, 3.0 Hz, 3H), 7.13 (d, J=2.1 Hz, 1H), 6.90-6.79 (m, 2H), 6.54 (dd, J=17.0, 10.2 Hz, 1H), 6.47 (d, J=8.9 Hz, 1H), 6.23 (dd, J=17.1, 2.1 Hz, 1H), 5.73 (dd, J=10.1, 2.1 Hz, 1H), 5.59 (d, J=6.2 Hz, 1H), 4.31-4.20 (m, 1H), 4.17 (d, J=6.1 Hz, 2H), 4.09 (s, 1H), 3.51 (s, 1H), 3.39 (dd, J=8.9, 2.4 Hz, 2H), 3.07 (d, J=9.0 Hz, 1H), 2.81 (d, J=9.6 Hz, 1H), 2.65 (dd, J=9.7, 2.3 Hz, 1H), 2.26 (s, 3H), 1.92 (d, J=11.2 Hz, 2H), 1.85-1.71 (m, 3H), 1.68 (d, J=9.2 Hz, 1H), 1.31 (dt, J=15.5, 11.6 Hz, 3H), 1.14 (t, J=13.5 Hz, 3H).

HRMS (ESI) calcd for C₃₅H₄₀N₈O₂[M+H]⁺, 605.3347; found, 605.3329.

### Example 7: N-(5-(3-benzyl-1-((1r,4r)-4-((5-cyanopyridin-2-yl)amino)cyclohexyl)ureido)-2-(4-(oxetan-3-yl)piperazin-1-yl)phenyl)acrylamide (YJZ6028)

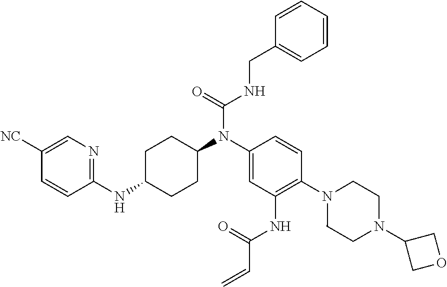 [SMILES: C=CC(=O)Nc1cc(N(C(=O)NCc2ccccc2)[C@H]2CC[C@H](Nc3ccc(C#N)cn3)CC2)ccc1N1CCN(C2COC2)CC1]

The synthetic method was similar to the method as shown in Example 1.

¹H NMR (400 MHz, DMSO-d₆) δ 9.02 (s, 1H), 8.31 (dd, J=2.3, 0.7 Hz, 1H), 7.96-7.87 (m, 1H), 7.60 (dd, J=8.9, 2.4 Hz, 1H), 7.49 (d, J=7.6 Hz, 1H), 7.31-7.21 (m, 3H), 7.21-7.13 (m, 3H), 6.93 (dd, J=8.4, 2.4 Hz, 1H), 6.68 (dd, J=17.0, 10.2 Hz, 1H), 6.47 (d, J=8.8 Hz, 1H), 6.27 (dd, J=17.0, 1.8 Hz, 1H), 5.78 (dd, J=8.8, 3.0 Hz, 2H), 4.58 (t, J=6.5 Hz, 2H), 4.49 (t, J=6.1 Hz, 2H), 4.32-4.22 (m, 1H), 4.17 (d, J=6.0 Hz, 2H), 3.53 (p, J=6.3 Hz, 2H), 3.51 (s, H) 2.90 (t, J=4.6 Hz, 4H), 1.99 (s, 1H), 1.92 (d, J=12.0 Hz, 2H), 1.78 (d, J=11.8 Hz, 2H), 1.32 (d, J=12.6 Hz, 2H), 1.24 (s, 2H), 1.19-1.11 (m, 2H).

HRMS (ESI) calcd for C₃₆H₄₂N₈O₃ [M+H]⁺, 635.3453; found, 635.3431.

### Example 8: N-(5-(3-benzyl-1-((1r,4r)-4-((5-cyanopyridin-2-yl)amino)cyclohexyl)ureido)-2-(4-(prop-2-yn-1-yl)piperazin-1-yl)phenyl)acrylamide (YJZ6085)

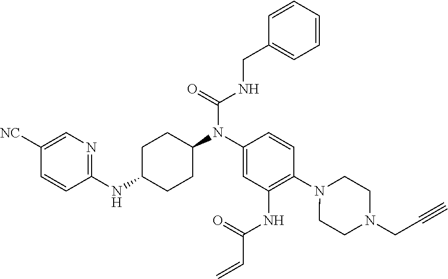 [SMILES: C#CCN1CCN(c2ccc(N(C(=O)NCc3ccccc3)[C@H]3CC[C@H](Nc4ccc(C#N)cn4)CC3)cc2NC(=O)C=C)CC1]

The synthetic method was similar to the method as shown in Example 1.

¹H NMR (400 MHz, Acetone-d₆) δ 8.89 (s, 1H), 8.37 (d, J=2.4 Hz, 1H), 8.29 (d, J=2.3 Hz, 1H), 7.56 (dd, J=8.8, 2.4 Hz, 1H), 7.34 (d, J=8.3 Hz, 1H), 7.30-7.22 (m, 4H), 7.18 (td, J=6.5, 5.9, 2.6 Hz, 1H), 7.01 (dd, J=8.4, 2.5 Hz, 1H), 6.73 (d, J=7.9 Hz, 1H), 6.60-6.49 (m, 2H), 6.36 (dd, J=16.9, 1.7 Hz, 1H), 5.79 (dd, J=10.2, 1.7 Hz, 1H), 5.28 (t, J=6.1 Hz, 1H), 4.54-4.44 (m, 1H), 4.32 (d, J=6.1 Hz, 2H), 3.70 (s, 1H), 3.39 (d, J=2.5 Hz, 2H), 3.12 (q, J=7.3 Hz, 2H), 2.96 (t, J=4.8 Hz, 3H), 2.78 (p, J=3.2, 2.7 Hz, 4H), 2.13-2.08 (m, 2H), 1.97-1.90 (m, 2H), 1.52-1.41 (m, 2H), 1.36 (m, 2H).

HRMS (ESI) calcd for C₃₆H₄₀N₈O₂[M+H]⁺, 617.3347; found, 617.3326.

### Example 9: N-(5-(3-benzyl-1-(((1r,4r)-4-((5-cyanopyridin-2-yl)amino)cyclohexyl)ureido)-2-(3-(dimethylamino)pyrrolidin-1-yl)phenyl)acrylamide (YJZ6082)

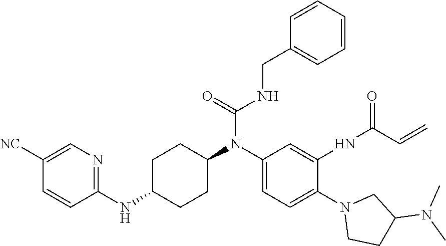 [SMILES: C=CC(=O)Nc1cc(N(C(=O)NCc2ccccc2)[C@H]2CC[C@H](Nc3ccc(C#N)cn3)CC2)ccc1N1CCC(N(C)C)C1]

The synthetic method was similar to the method as shown in Example 1.

¹H NMR (400 MHz, DMSO-d₆) δ 9.46 (s, 1H), 8.32 (d, J=2.4 Hz, 1H), 7.61 (dd, J=8.9, 2.4 Hz, 1H), 7.50 (d, J=7.6 Hz, 1H), 7.31-7.23 (m, 2H), 7.21-7.13 (m, 4H), 6.89 (s, 2H), 6.56 (dd, J=17.0, 10.2 Hz, 1H), 6.47 (d, J=8.9 Hz, 1H), 6.24 (dd, J=17.0, 2.1 Hz, 1H), 5.74 (dd, J=10.0, 2.1 Hz, 1H), 5.57 (d, J=6.2 Hz, 1H), 4.26 (t, J=3.5 Hz, 1H), 4.17 (d, J=6.2 Hz, 2H), 3.50 (s, 1H), 3.21 (t, J=8.9 Hz, 4H), 2.66 (t, J=8.0 Hz, 1H), 2.16 (s, 6H), 2.13-2.05 (m, 1H), 1.92 (s, 2H), 1.81-1.68 (m, 3H), 1.30 (t, J=12.6 Hz, 2H), 1.19-1.05 (m, 2H).

HRMS (ESI) calcd for C₃₅H₄₂N₈O₂ [M+H]⁺, 607.3503; found, 607.3480.

### Example 10: N-(5-(3-benzyl-1-((1r,4r)-4-((5-cyanopyridin-2-yl)amino)cyclohexyl)ureido)-2-(3-(dimethylamino)azetidin-1-yl)phenyl)acrylamide (YJZ6077)

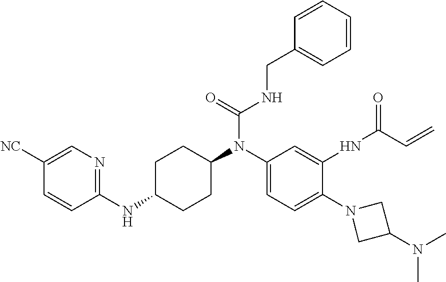 [SMILES: C=CC(=O)Nc1cc(N(C(=O)NCc2ccccc2)[C@H]2CC[C@H](Nc3ccc(C#N)cn3)CC2)ccc1N1CC(N(C)C)C1]

The synthetic method was similar to the method as shown in Example 1.

¹H NMR (400 MHz, DMSO-d₆) δ 9.38 (s, 1H), 8.33 (d, J=2.3 Hz, 1H), 7.61 (dd, J=8.9, 2.4 Hz, 1H), 7.49 (d, J=7.6 Hz, 1H), 7.30-7.23 (m, 2H), 7.20-7.14 (m, 3H), 7.11 (d, J=2.4 Hz, 1H), 6.90 (dd, J=8.6, 2.4 Hz, 1H), 6.61 (d, J=8.5 Hz, 1H), 6.55 (dd, J=17.0, 10.2 Hz, 1H), 6.47 (d, J=8.9 Hz, 1H), 6.24 (dd, J=17.0, 2.1 Hz, 1H), 5.74 (dd, J=10.2, 2.1 Hz, 1H), 5.59 (t, J=6.1 Hz, 1H), 4.31-4.20 (m, 1H), 4.17 (d, J=6.0 Hz, 2H), 3.98 (t, J=7.2 Hz, 2H), 3.60 (t, J=6.7 Hz, 2H), 3.50 (s, 1H), 3.12 (d, J=14.5 Hz, H), 2.11 (s, 6H), 1.96-1.85 (m, 2H), 1.76 (d, J=12.0 Hz, 2H), 1.39-1.23 (m, 2H), 1.14 (ddd, J=25.5, 13.6, 8.9 Hz, 2H).

HRMS (ESI) calcd for C₃₄H₄₀N₈O₂[M+H]⁺, 593.3347; found, 593.3329.

### Example 11: N-(5-(3-benzyl-1-((1r,4r)-4-((5-cyanopyridin-2-yl)amino)cyclohexyl)ureido)-2-bromophenyl)acrylamide (YJZ6048)

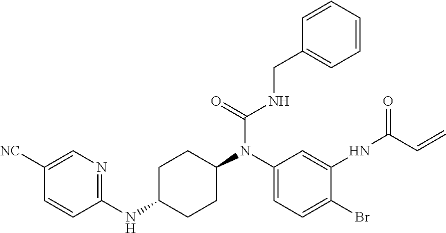 [SMILES: C=CC(=O)Nc1cc(N(C(=O)NCc2ccccc2)[C@H]2CC[C@H](Nc3ccc(C#N)cn3)CC2)ccc1Br]

The synthetic method was similar to the method as shown in Example 1.

¹H NMR (400 MHz, DMSO-d₆) δ 9.67 (s, 1H), 8.32 (d, J=2.4 Hz, 1H), 7.73 (d, J=8.4 Hz, 1H), 7.61 (dd, J=9.0, 2.4 Hz, 2H), 7.48 (d, J=7.6 Hz, 1H), 7.30-7.24 (m, 2H), 7.23-7.14 (m, 3H), 6.96 (dd, J=8.5, 2.5 Hz, 1H), 6.68 (dd, J=17.0, 10.2 Hz, 1H), 6.48 (d, J=8.9 Hz, 1H), 6.31 (dd, J=17.0, 2.0 Hz, 1H), 6.11 (t, J=5.9 Hz, 1H), 5.82 (dd, J=10.2, 2.0 Hz, 1H), 4.34-4.22 (m, 1H), 4.16 (d, J=6.0 Hz, 2H), 3.52 (s, 1H), 1.93 (d, J=12.1 Hz, 2H), 1.79 (d, J=12.1 Hz, 2H), 1.32 (q, J=12.3 Hz, 2H), 1.24-1.10 (m, 2H).

HRMS (ESI) calcd for C₂₉H₂₉BrN₆O₂ [M+H]⁺, 573.1608; found, 573.1618.

### Example 12: N-(5-(3-benzyl-1-(((1r,4S)-4-((5-cyanopyridin-2-yl)amino)cyclohexyl)ureido)-2-((S)-3,4-dimethylpiperazin-1-yl)phenyl)acrylamide(YJZ6093)

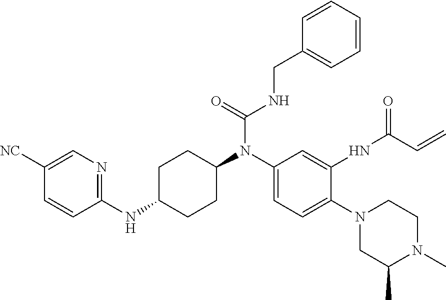 [SMILES: C=CC(=O)Nc1cc(N(C(=O)NCc2ccccc2)[C@H]2CC[C@H](Nc3ccc(C#N)cn3)CC2)ccc1N1CCN(C)[C@@H](C)C1]

The synthetic method was similar to the method as shown in Example 1.

¹H NMR (400 MHz, DMSO-d₆) δ 9.02 (s, 1H), 8.31 (d, J=2.3 Hz, 1H), 7.91 (s, 1H), 7.60 (dd, J=8.9, 2.3 Hz, 1H), 7.49 (d, J=7.6 Hz, 1H), 7.30-7.24 (m, 2H), 7.21 (d, J=8.4 Hz, 1H), 7.20-7.15 (m, 3H), 6.92 (dd, J=8.4, 2.4 Hz, 1H), 6.65 (dd, J=16.9, 10.2 Hz, 1H), 6.47 (d, J=8.9 Hz, 1H), 6.27 (dd, J=17.0, 1.9 Hz, 1H), 5.78 (td, J=9.6, 3.9 Hz, 2H), 4.33-4.22 (m, 1H), 4.17 (d, J=6.0 Hz, 2H), 3.51 (s, 1H), 2.95-2.75 (m, 4H), 2.45 (d, J=10.6 Hz, 2H), 2.37 (d, J=9.4 Hz, 1H), 2.25 (s, 3H), 1.92 (d, J=12.0 Hz, 2H), 1.78 (d, J=12.0 Hz, 2H), 1.32 (q, J=12.5 Hz, 2H), 1.16 (t, J=12.8 Hz, 2H), 1.01 (d, J=6.1 Hz, 3H).

HRMS (ESI) calcd for C₃₅H₄₂N₈O₂ [M+H]⁺, 607.3503; found, 607.3521.

### Example 13: N-(5-(3-benzyl-1-(((1r,4S)-4-((5-cyanopyridin-2-yl)amino)cyclohexyl)ureido)-2-((S)-3-methylpiperazin-1-yl)phenyl)acrylamide (YJZ8027)

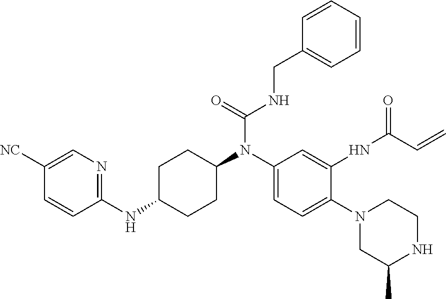 [SMILES: C=CC(=O)Nc1cc(N(C(=O)NCc2ccccc2)[C@H]2CC[C@H](Nc3ccc(C#N)cn3)CC2)ccc1N1CCN[C@@H](C)C1]

The synthetic method was similar to the method as shown in Example 1.

¹H NMR (400 MHz, DMSO-d₆) δ 9.03 (s, 1H), 8.31 (d, J=2.3 Hz, 1H), 7.93 (s, 1H), 7.60 (dd, J=8.9, 2.4 Hz, 1H), 7.49 (d, J=7.6 Hz, 1H), 7.26 (q, J=6.8, 6.0 Hz, 2H), 7.22-7.12 (m, 4H), 6.91 (dd, J=8.4, 2.5 Hz, 1H), 6.66 (dd, J=16.9, 10.2 Hz, 1H), 6.47 (d, J=8.9 Hz, 1H), 6.27 (dd, J=17.0, 1.9 Hz, 1H), 5.82-5.72 (m, 2H), 4.27 (s, 1H), 4.17 (d, J=6.0 Hz, 2H), 3.47 (d, J=7.1 Hz, 1H), 3.03-2.81 (m, 4H), 2.68-2.57 (m, 2H), 2.28 (t, J=10.3 Hz, 1H), 1.92 (d, J=12.0 Hz, 2H), 1.78 (d, J=11.9 Hz, 2H), 1.38-1.25 (m, 2H), 1.20-1.08 (m, 2H), 0.97 (d, J=6.4 Hz, 3H).

HRMS (ESI) calcd for C₃₄H₄₀N₈O₂[M+H]⁺, 593.3347; found, 593.3333.

### Example 14: N-(5-(3-benzyl-1-(((1r,4r)-4-((5-cyanopyridin-2-yl)amino)cyclohexyl)ureido)-2-(1-methyl-6-oxo-1,6-dihydropyridin-3-yl)phenyl)acrylamide (YJZ5028)

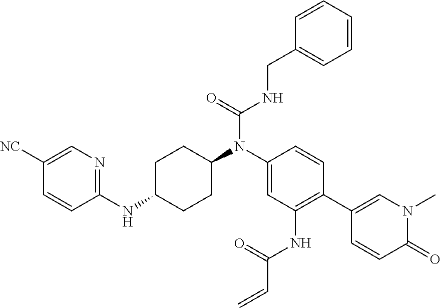 [SMILES: C=CC(=O)Nc1cc(N(C(=O)NCc2ccccc2)[C@H]2CC[C@H](Nc3ccc(C#N)cn3)CC2)ccc1-c1ccc(=O)n(C)c1]

The synthetic method was similar to the method as shown in Example 1.

¹H NMR (400 MHz, DMSO-d₆) δ 9.61 (s, 1H), 8.32 (d, J=2.3 Hz, 1H), 7.88 (d, J=2.6 Hz, 1H), 7.60 (q, J=9.6, 7.4 Hz, 3H), 7.45 (dd, J=9.3, 2.6 Hz, 1H), 7.39 (d, J=8.1 Hz, 1H), 7.28 (t, J=7.5 Hz, 2H), 7.24-7.14 (m, 3H), 7.06 (dd, J=8.1, 2.2 Hz, 1H), 6.47 (dd, J=13.9, 9.2 Hz, 3H), 6.24 (dd, J=17.0, 2.2 Hz, 1H), 5.98 (s, 1H), 5.74 (d, J=10.2 Hz, 1H), 4.30 (t, J=11.9 Hz, 1H), 4.19 (d, J=6.0 Hz, 2H), 3.65-3.55 (m, 1H), 3.48 (s, 3H), 1.93 (d, J=12.0 Hz, 2H), 1.82 (d, J=11.7 Hz, 2H), 1.31 (td, J=15.4, 14.6, 7.1 Hz, 4H).

HRMS (ESI) calcd for C₃₅H₃₅N₇O₃ [M+H]⁺, 602.2874; found, 602.2853.

### Example 15: N-(3-(3-benzyl-1-((1r,4r)-4-((5-cyanopyridin-2-yl)amino)cyclohexyl)ureido)phenyl)acrylamide (YJZ4156)

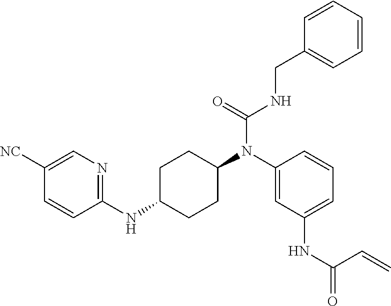 [SMILES: C=CC(=O)Nc1cccc(N(C(=O)NCc2ccccc2)[C@H]2CC[C@H](Nc3ccc(C#N)cn3)CC2)c1]

The synthetic method was similar to the method as shown in Example 1.

¹H NMR (400 MHz, DMSO-d₆) δ 10.30 (s, 1H), 8.31 (d, J=2.3 Hz, 1H), 7.79-7.69 (m, 1H), 7.61 (dd, J=8.9, 2.4 Hz, 1H), 7.56 (t, J=2.0 Hz, 1H), 7.55-7.48 (m, 1H), 7.41 (t, J=8.0 Hz, 1H), 7.31-7.23 (m, 2H), 7.23-7.13 (m, 3H), 6.90 (dt, J=7.8, 1.3 Hz, 1H), 6.51-6.39 (m, 2H), 6.29 (dd, J=17.0, 2.1 Hz, 1H), 5.89 (t, J=6.0 Hz, 1H), 5.79 (dd, J=10.0, 2.1 Hz, 1H), 4.29 (td, J=9.9, 8.2, 5.7 Hz, 1H), 4.16 (d, J=6.0 Hz, 2H), 3.51 (s, 1H), 1.93 (d, J=12.0 Hz, 2H), 1.79 (d, J=11.9 Hz, 2H), 1.32 (q, J=12.3 Hz, 2H), 1.16 (td, J=15.6, 14.5, 7.2 Hz, 2H).

HRMS (ESI) calcd for C₂₉H₃₀N₆O₂[M+H]⁺, 495.2503; found, 495.2485.

### Example 16: (E)-N-(5-(3-benzyl-1-(((1r,4r)-4-((5-cyanopyridin-2-yl)amino)cyclohexyl)ureido)-2-(1-methyl-6-oxo-1,6-dihydropyridin-3-yl)phenyl)-4-(dimethylamino)but-2-enamide (YJZ5078)

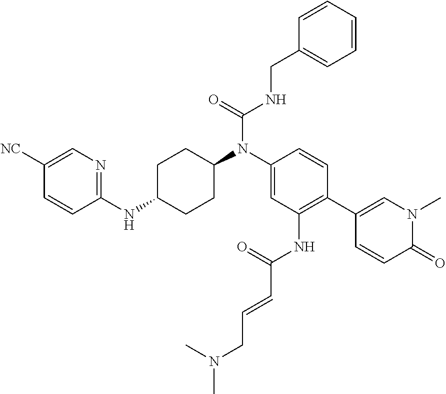 [SMILES: CN(C)C/C=C/C(=O)Nc1cc(N(C(=O)NCc2ccccc2)[C@H]2CC[C@H](Nc3ccc(C#N)cn3)CC2)ccc1-c1ccc(=O)n(C)c1]

The synthetic method was similar to the method as shown in Example 1.

¹H NMR (400 MHz, DMSO-d₆) δ 9.56 (s, 1H), 8.32 (d, J=2.4 Hz, 1H), 7.86 (d, J=2.7 Hz, 1H), 7.67-7.58 (m, 2H), 7.53 (d, J=7.6 Hz, 1H), 7.44 (dd, J=9.4, 2.7 Hz, 1H), 7.38 (dd, J=8.0, 2.9 Hz, 1H), 7.33-7.25 (m, 2H), 7.25-7.15 (m, 3H), 7.11-7.01 (m, 1H), 6.72 (d, J=15.3 Hz, 1H), 6.51-6.43 (m, 2H), 6.40 (d, J=15.4 Hz, 1H), 5.95 (s, 1H), 4.35-4.25 (m, 1H), 4.19 (d, J=6.0 Hz, 2H), 3.48 (d, J=2.4 Hz, 4H), 1.93 (d, J=12.9 Hz, 2H), 1.82 (d, J=12.4 Hz, 2H), 1.40-1.28 (m, 2H), 1.19 (m, 2H).

HRMS (ESI) calcd for C₃₈H₄₂N₈O₃ [M+H]⁺, 659.3453; found, 659.3456.

### Example 17: N-(5-(3-benzyl-1-(((1r,4S)-4-((5-cyanopyrimidin-2-yl)amino)cyclohexyl)ureido)-2-((S)-3,4-dimethylpiperazin-1-yl)phenyl)acrylamide (YJZ7007)

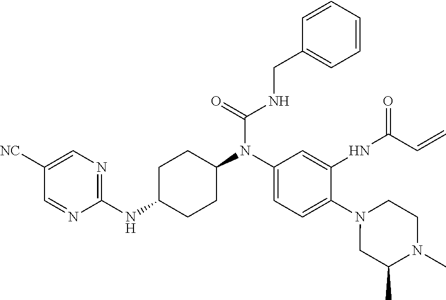 [SMILES: C=CC(=O)Nc1cc(N(C(=O)NCc2ccccc2)[C@H]2CC[C@H](Nc3ncc(C#N)cn3)CC2)ccc1N1CCN(C)[C@@H](C)C1]

The synthetic method was similar to the method as shown in Example 1.

¹H NMR (400 MHz, DMSO-d₆) δ 9.04 (s, 1H), 8.62 (q, J=3.0 Hz, 2H), 8.28 (d, J=8.0 Hz, 1H), 7.94-7.84 (m, 1H), 7.31-7.24 (m, 2H), 7.23-7.13 (m, 4H), 6.92 (dd, J=8.4, 2.4 Hz, 1H), 6.66 (dd, J=16.9, 10.2 Hz, 1H), 6.27 (dd, J=16.9, 1.9 Hz, 1H), 5.88-5.75 (m, 2H), 4.31-4.20 (m, 1H), 4.17 (d, J=6.0 Hz, 2H), 3.57-3.45 (m, 1H), 2.97-2.75 (m, 4H), 2.49-2.41 (m, 2H), 2.36 (td, J=6.4, 2.9 Hz, 1H), 2.25 (s, 3H), 1.91-1.83 (m, 2H), 1.82-1.72 (m, 2H), 1.50-1.35 (m, 2H), 1.12 (dt, J=12.6, 3.8 Hz, 2H), 1.00 (d, J=6.1 Hz, 3H).

HRMS (ESI) calcd for C₃₄H₄₁N₉O₂ [M+H]⁺, 608.3456; found, 608.3463.

### Example 18: N-(5-((((1r,4r)-4-)((5-cyanopyridin-2-yl)amino)cyclohexyl)amino)-2-(4-(dimethylamino)piperidin-1-yl)phenyl)acrylamide (YJZ6109)

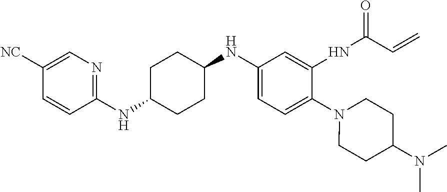 [SMILES: C=CC(=O)Nc1cc(N[C@H]2CC[C@H](Nc3ccc(C#N)cn3)CC2)ccc1N1CCC(N(C)C)CC1]

The synthetic method was similar to the method as shown in Example 1.

¹H NMR (400 MHz, DMSO-d₆) δ 8.96 (s, 1H), 8.38 (d, J=2.3 Hz, 1H), 7.70-7.59 (m, 1H), 7.53 (d, J=7.6 Hz, 2H), 6.94 (d, J=8.6 Hz, 1H), 6.60 (dd, J=16.9, 10.2 Hz, 1H), 6.53 (d, J=8.9 Hz, 1H), 6.29 (dd, J=8.7, 2.7 Hz, 1H), 6.21 (dd, J=16.9, 1.9 Hz, 1H), 5.80-5.70 (m, 1H), 5.31 (d, J=8.1 Hz, 1H), 3.77 (s, 1H), 3.11 (d, J=7.6 Hz, 1H), 2.82 (d, J=11.3 Hz, 2H), 2.61-2.52 (m, 2H), 2.20 (s, 6H), 2.13 (tt, J=10.7, 3.6 Hz, 1H), 2.05-1.92 (m, 4H), 1.80 (d, J=12.2 Hz, 2H), 1.63 (tt, J=12.9, 6.5 Hz, 2H), 1.37-1.27 (m, 2H), 1.23 (dd, J=9.8, 3.7 Hz, 2H).

HRMS (ESI) calcd for C₂₈H₃₇N₇O [M+H]⁺, 488.3132; found, 488.3134.

### Example 19: N-(5-(1-((1r,4S)-4-((5-cyanopyridin-2-yl)amino)cyclohexyl)-3-(cyclopropylmethyl)ureido)-2-((S)-3,4-dimethylpiperazin-1-yl)phenyl)acrylamide (YJZ1025)

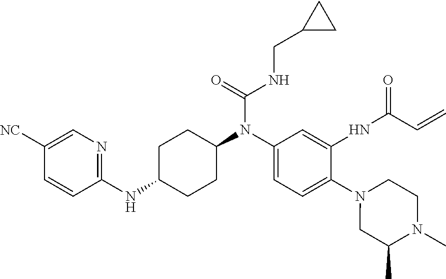 [SMILES: C=CC(=O)Nc1cc(N(C(=O)NCC2CC2)[C@H]2CC[C@H](Nc3ccc(C#N)cn3)CC2)ccc1N1CCN(C)[C@@H](C)C1]

The synthetic method was similar to the method as shown in Example 1.

¹H NMR (400 MHz, DMSO-d₆) δ 9.03 (s, 1H), 8.31 (d, J=2.3 Hz, 1H), 7.88-7.78 (m, 1H), 7.61 (dd, J=8.9, 2.4 Hz, 1H), 7.49 (d, J=7.6 Hz, 1H), 7.20 (d, J=8.4 Hz, 1H), 6.88 (dd, J=8.4, 2.5 Hz, 1H), 6.64 (dd, J=16.9, 10.2 Hz, 1H), 6.47 (d, J=8.9 Hz, 1H), 6.26 (dd, J=16.9, 1.9 Hz, 1H), 5.79 (dd, J=10.2, 1.9 Hz, 1H), 5.09 (t, J=5.7 Hz, 1H), 4.26 (tt, J=11.2, 3.0 Hz, 2H), 3.48 (s, 1H), 2.97-2.74 (m, 6H), 2.45 (d, J=10.7 Hz, 2H), 2.39-2.32 (m, 1H), 2.25 (s, 3H), 1.91 (d, J=12.2 Hz, 2H), 1.76 (d, J=12.0 Hz, 2H), 1.32 (q, J=12.5 Hz, 2H), 1.12 (q, J=11.9, 11.5 Hz, 3H), 1.01 (d, J=6.1 Hz, 3H), 0.88-0.81 (m, 1H), 0.31-0.24 (m, 2H), 0.08-0.01 (m, 2H).

HRMS (ESI) calcd for C₃₂H₄₂N₈O₂ [M+H]⁺, 571.3503; found, 571.3491

### Example 20: N-(5-(1-((1r,4S)-4-((5-cyanopyridin-2-yl)amino)cyclohexyl)-3-(oxetanyl-3-ylmethyl)ureido)-2-((S)-3,4-dimethylpiperazin-1-yl)phenyl)acrylamide (YJZ1031)

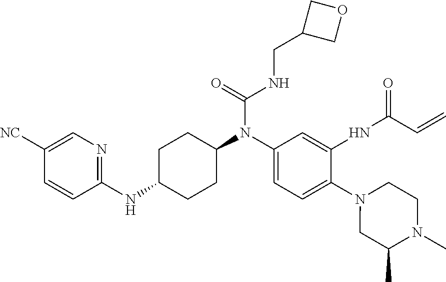 [SMILES: C=CC(=O)Nc1cc(N(C(=O)NCC2COC2)[C@H]2CC[C@H](Nc3ccc(C#N)cn3)CC2)ccc1N1CCN(C)[C@@H](C)C1]

The synthetic method was similar to the method as shown in Example 1.

¹H NMR (400 MHz, DMSO-d₆) δ 9.01 (s, 1H), 8.30 (d, J=2.3 Hz, 1H), 7.80 (s, 1H), 7.60 (dd, J=8.8, 2.4 Hz, 1H), 7.48 (d, J=7.6 Hz, 1H), 7.19 (d, J=8.4 Hz, 1H), 6.85 (dd, J=8.4, 2.4 Hz, 1H), 6.64 (dd, J=16.9, 10.2 Hz, 1H), 6.47 (d, J=9.0 Hz, 1H), 6.25 (dd, J=17.0, 1.8 Hz, 1H), 5.79 (dd, J=10.2, 1.9 Hz, 1H), 5.44 (t, J=5.9 Hz, 1H), 4.50 (ddd, J=7.7, 5.9, 1.7 Hz, 2H), 4.22 (q, J=6.1 Hz, 3H), 3.47 (s, 1H), 3.22 (t, J=6.3 Hz, 2H), 3.01-2.76 (m, 5H), 2.49-2.42 (m, 2H), 2.40-2.30 (m, 1H), 2.25 (s, 3H), 1.91 (d, J=12.9 Hz, 2H), 1.75 (d, J=12.9 Hz, 2H), 1.31 (q, J=13.4, 12.7 Hz, 2H), 1.11 (q, J=10.2, 7.9 Hz, 2H), 1.01 (d, J=6.1 Hz, 3H).

HRMS (ESI) calcd for C₃₂H₄₂N₈O₃ [M+H]⁺, 587.3453; found, 571.3440

### Example 21: N-(5-(1-((1r,4S)-4-((5-cyanopyridin-2-yl)amino)cyclohexyl)-3-((1-methyl-1H-pyrazol-4-yl)methyl)ureido)-2-((S)-3,4-dimethylpiperazin-1-yl)phenyl)acrylamide (YJZ1033)

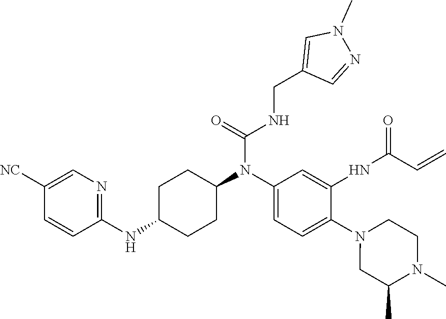 [SMILES: C=CC(=O)Nc1cc(N(C(=O)NCc2cnn(C)c2)[C@H]2CC[C@H](Nc3ccc(C#N)cn3)CC2)ccc1N1CCN(C)[C@@H](C)C1]

The synthetic method was similar to the method as shown in Example 1.

¹H NMR (400 MHz, DMSO-d₆) δ 9.02 (s, 1H), 8.30 (d, J=2.4 Hz, 1H), 7.82 (s, 1H), 7.60 (dd, J=8.9, 2.4 Hz, 1H), 7.49 (d, J=7.6 Hz, 1H), 7.44 (s, 1H), 7.23-7.13 (m, 2H), 6.87 (dd, J=8.4, 2.5 Hz, 1H), 6.64 (dd, J=16.9, 10.3 Hz, 1H), 6.47 (d, J=8.8 Hz, 1H), 6.26 (dd, J=17.0, 1.9 Hz, 1H), 5.79 (dd, J=10.2, 1.9 Hz, 1H), 5.40 (t, J=5.7 Hz, 1H), 4.25 (d, J=11.5 Hz, 1H), 3.97 (d, J=5.8 Hz, 2H), 3.75 (s, 3H), 3.49 (s, 1H), 2.95-2.76 (m, 4H), 2.47-2.41 (m, 2H), 2.38-2.31 (m, 1H), 2.25 (s, 3H), 1.91 (d, J=12.4 Hz, 2H), 1.76 (d, J=12.0 Hz, 2H), 1.31 (q, J=12.5 Hz, 2H), 1.12 (q, J=12.7 Hz, 2H), 1.01 (d, J=6.1 Hz, 3H).

HRMS (ESI) calcd for C₃₃H₄₂N₁₀O₂ [M+H]⁺, 611.3565; found, 611.3550

### Example 22: N-(5-(1-((1r,4S)-4-((5-cyanopyridin-2-yl)amino)cyclohexyl)-3-cyclopropylureido)-2-((S)-3,4-dimethylpiperazin-1-yl)phenyl)acrylamide (YJZ9173)

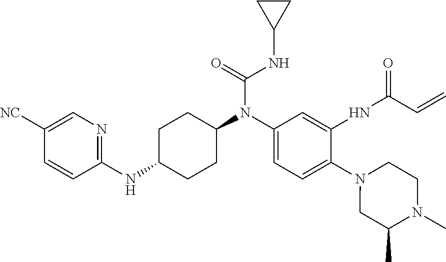 [SMILES: C=CC(=O)Nc1cc(N(C(=O)NC2CC2)[C@H]2CC[C@H](Nc3ccc(C#N)cn3)CC2)ccc1N1CCN(C)[C@@H](C)C1]

The synthetic method was similar to the method as shown in Example 1.

¹H NMR (400 MHz, DMSO-d₆) δ 9.00 (s, 1H), 8.30 (d, J=2.3 Hz, 1H), 7.78 (s, 1H), 7.60 (dd, J=8.9, 2.4 Hz, 1H), 7.47 (d, J=7.8 Hz, 1H), 7.17 (d, J=8.4 Hz, 1H), 6.83 (dd, J=8.4, 2.4 Hz, 1H), 6.63 (dd, J=16.9, 10.3 Hz, 1H), 6.48 (d, J=8.9 Hz, 1H), 6.25 (dd, J=16.9, 1.9 Hz, 1H), 5.79 (dd, J=10.2, 1.9 Hz, 1H), 5.14 (d, J=2.9 Hz, 1H), 4.24 (tt, J=12.0, 3.7 Hz, 1H), 3.48 (s, 1H), 2.96-2.75 (m, 4H), 2.49-2.41 (m, 3H), 2.35 (ddd, J=9.2, 6.0, 2.7 Hz, 1H), 2.25 (s, 3H), 1.91 (d, J=11.9 Hz, 2H), 1.76 (d, J=11.9 Hz, 2H), 1.32 (q, J=13.1, 12.5 Hz, 2H), 1.12 (q, J=13.1, 12.5 Hz, 2H, 2H), 1.01 (d, J=6.1 Hz, 3H), 0.51-0.44 (m, 2H), 0.32-0.24 (m, 2H).

### Example 23: N-(5-(1-((1r,4S)-4-((5-cyanopyridin-2-yl)amino)cyclohexyl)-3-(2-fluorobenzyl)ureido)-2-((S)-3,4-dimethylpiperazin-1-yl)phenyl)acrylamide (YJZ9179)

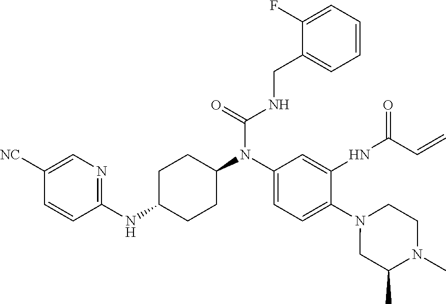 [SMILES: C=CC(=O)Nc1cc(N(C(=O)NCc2ccccc2F)[C@H]2CC[C@H](Nc3ccc(C#N)cn3)CC2)ccc1N1CCN(C)[C@@H](C)C1]

The synthetic method was similar to the method as shown in Example 1.

¹H NMR (400 MHz, DMSO-d₆) δ 9.02 (s, 1H), 8.31 (d, J=2.9 Hz, 1H), 7.93 (s, 1H), 7.60 (dd, J=8.9, 2.4 Hz, 1H), 7.47 (d, J=7.6 Hz, 1H), 7.28-7.20 (m, 3H), 7.15 (td, J=7.3, 1.3 Hz, 1H), 7.09 (ddd, J=9.9, 8.6, 1.3 Hz, 1H), 6.94 (dd, J=8.4, 2.5 Hz, 1H), 6.66 (dd, J=16.9, 10.2 Hz, 1H), 6.47 (d, J=8.9 Hz, 1H), 6.27 (dd, J=17.0, 1.8 Hz, 1H), 5.84-5.73 (m, 2H), 4.31-4.24 (m, 1H), 4.22 (d, J=6.0 Hz, 2H), 3.50 (s, 1H), 2.96-2.77 (m, 4H), 2.48-2.43 (m, 2H), 2.41-2.32 (m, 1H), 1.92 (d, J=11.6 Hz, 2H), 1.78 (d, J=11.3 Hz, 2H), 1.32 (q, J=12.2 Hz, 2H), 1.15 (q, J=12.6 Hz, 2H), 1.01 (d, J=6.1 Hz, 3H).

### Example 24: N-(5-(1-((1r,4S)-4-((5-cyanopyridin-2-yl)amino)cyclohexyl)-3-(3-fluorobenzyl)ureido)-2-((S)-3,4-dimethylpiperazin-1-yl)phenyl)acrylamide (YJZ9177)

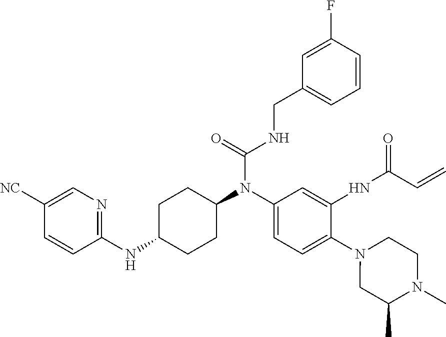 [SMILES: C=CC(=O)Nc1cc(N(C(=O)NCc2cccc(F)c2)[C@H]2CC[C@H](Nc3ccc(C#N)cn3)CC2)ccc1N1CCN(C)[C@@H](C)C1]

The synthetic method was similar to the method as shown in Example 1.

¹H NMR (400 MHz, DMSO-d₆) δ 9.01 (s, 1H), 8.31 (d, J=2.4 Hz, 1H), 7.91 (s, 1H), 7.60 (dd, J=8.9, 2.4 Hz, 1H), 7.47 (d, J=7.6 Hz, 1H), 7.31 (td, J=7.9, 6.1 Hz, 1H), 7.22 (d, J=8.4 Hz, 1H), 7.06-7.01 (m, 1H), 7.01-6.94 (m, 2H), 6.92 (dd, J=8.4, 2.5 Hz, 1H), 6.65 (dd, J=16.9, 10.2 Hz, 1H), 6.47 (d, J=8.9 Hz, 1H), 6.27 (dd, J=16.9, 1.9 Hz, 1H), 5.87 (t, J=6.1 Hz, 1H), 5.79 (dd, J=10.1, 1.9 Hz, 1H), 4.27 (ddd, J=11.9, 8.2, 3.7 Hz, 1H), 4.17 (d, J=6.0 Hz, 2H), 3.50 (s, 1H), 2.97-2.75 (m, 4H), 2.48-2.43 (m, 2H), 2.41-2.32 (m, 1H), 2.25 (s, 3H), 1.97-1.87 (m, 2H), 1.78 (d, J=12.0 Hz, 2H), 1.32 (q, J=12.3, 11.8 Hz, 2H), 1.15 (q, J=15.4, 14.3 Hz, 2H), 1.01 (d, J=6.1 Hz, 3H).

### Example 25: N-(5-(1-((1r,4S)-4-((5-cyanopyridin-2-yl)amino)cyclohexyl)-3-(4-fluorobenzyl)ureido)-2-((S)-3,4-dimethylpiperazin-1-yl)phenyl)acrylamide (YJZ9166)

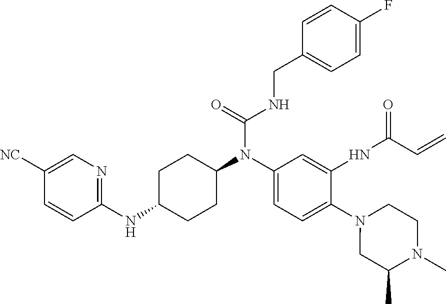 [SMILES: C=CC(=O)Nc1cc(N(C(=O)NCc2ccc(F)cc2)[C@H]2CC[C@H](Nc3ccc(C#N)cn3)CC2)ccc1N1CCN(C)[C@@H](C)C1]

The synthetic method was similar to the method as shown in Example 1.

¹H NMR (400 MHz, DMSO-d₆) δ 9.01 (s, 1H), 8.30 (d, J=2.3 Hz, 1H), 7.89 (s, 1H), 7.60 (dd, J=8.9, 2.3 Hz, 1H), 7.48 (d, J=7.6 Hz, 1H), 7.22 (dd, J=8.3, 6.3 Hz, 3H), 7.08 (t, J=8.8 Hz, 2H), 6.90 (dd, J=8.4, 2.5 Hz, 1H), 6.65 (dd, J=16.9, 10.2 Hz, 1H), 6.47 (d, J=8.9 Hz, 1H), 6.27 (dd, J=17.0, 1.8 Hz, 1H), 5.90-5.73 (m, 2H), 4.26 (td, J=10.1, 8.3, 5.9 Hz, 1H), 4.14 (d, J=5.9 Hz, 2H), 3.49 (s, 1H), 2.96-2.76 (m, 4H), 2.49-2.42 (m, 2H), 2.40-2.32 (m, 1H), 2.25 (s, 3H), 1.92 (d, J=10.1 Hz, 2H), 1.77 (d, J=10.9 Hz, 2H), 1.32 (q, J=13.5, 11.7 Hz, 2H), 1.14 (q, J=12.2 Hz, 2H), 1.01 (d, J=6.1 Hz, 3H).

### Example 26: N-(5-(1-((1r,4S)-4-((5-cyanopyridin-2-yl)amino)cyclohexyl)-3-((S)-1-phenylethyl)ureido)-2-((S)-3,4-dimethylpiperazin-1-yl)phenyl)acrylamide (YJZ9170)

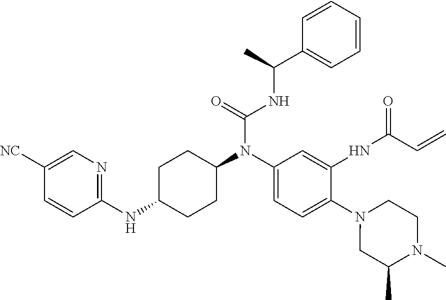 [SMILES: C=CC(=O)Nc1cc(N(C(=O)N[C@@H](C)c2ccccc2)[C@H]2CC[C@H](Nc3ccc(C#N)cn3)CC2)ccc1N1CCN(C)[C@@H](C)C1]

The synthetic method was similar to the method as shown in Example 1.

¹H NMR (400 MHz, DMSO-d₆) δ 9.04 (s, 1H), 8.30 (s, 1H), 7.86 (s, 1H), 7.60 (d, J=8.9 Hz, 1H), 7.45 (d, J=7.5 Hz, 1H), 7.34-7.14 (m, 6H), 6.92 (d, J=8.4 Hz, 1H), 6.64 (dd, J=17.0, 10.3 Hz, 1H), 6.47 (d, J=8.9 Hz, 1H), 6.28 (d, J=17.0 Hz, 1H), 5.80 (d, J=10.3 Hz, 1H), 5.17 (d, J=7.9 Hz, 1H), 4.88-4.76 (m, 1H), 4.23 (t, J=12.0 Hz, 1H), 3.49 (s, 1H), 3.01-2.72 (m, 4H), 2.48-2.41 (m, 2H), 2.36 (s, 1H), 2.25 (s, 3H), 1.91 (t, J=11.2 Hz, 2H), 1.80 (d, J=11.7 Hz, 1H), 1.71 (d, J=11.8 Hz, 1H), 1.30 (s, 2H), 1.24 (d, J=6.7 Hz, 3H), 1.19-1.07 (m, 2H), 1.02 (d, J=6.1 Hz, 3H).

### Example 27: N-(5-(1-((1r,4R)-4-((5-cyanopyridin-2-yl)amino)cyclohexyl)-3-((R)-1-phenylethyl)ureido)-2-((S)-3,4-dimethylpiperazin-1-yl)phenyl)acrylamide (YJZ9176)

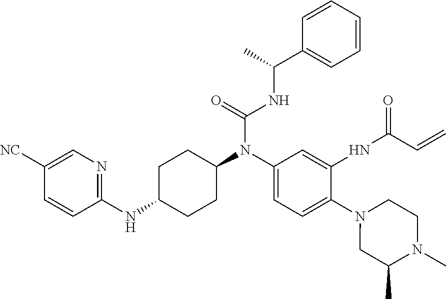 [SMILES: C=CC(=O)Nc1cc(N(C(=O)N[C@H](C)c2ccccc2)[C@H]2CC[C@H](Nc3ccc(C#N)cn3)CC2)ccc1N1CCN(C)[C@@H](C)C1]

The synthetic method was similar to the method as shown in Example 1.

¹H NMR (400 MHz, DMSO-d₆) δ 9.04 (s, 1H), 8.30 (d, J=2.4 Hz, 1H), 7.86 (s, 1H), 7.60 (dd, J=8.9, 2.4 Hz, 1H), 7.46 (d, J=7.6 Hz, 1H), 7.30-7.15 (m, 6H), 6.92 (dd, J=8.4, 2.5 Hz, 1H), 6.65 (dd, J=17.0, 10.2 Hz, 1H), 6.47 (d, J=8.9 Hz, 1H), 6.28 (dd, J=16.9, 1.9 Hz, 1H), 5.80 (dd, J=10.2, 1.9 Hz, 1H), 5.18 (d, J=8.0 Hz, 1H), 4.82 (p, J=7.1 Hz, 1H), 4.29-4.18 (m, 1H), 3.49 (s, 1H), 2.99-2.74 (m, 4H), 2.49-2.42 (m, 2H), 2.41-2.31 (m, 1H), 2.26 (s, 3H), 1.91 (t, J=10.7 Hz, 2H), 1.80 (d, J=11.9 Hz, 1H), 1.71 (d, J=12.4 Hz, 1H), 1.36-1.27 (m, 2H), 1.24 (d, J=7.0 Hz, 3H), 1.17-1.07 (m, 2H), 1.01 (d, J=6.1 Hz, 3H).

### Example 28: N-(5-(N-((1r,4r)-4-((5-cyanopyridin-2-yl)amino)cyclohexyl)methylsulfonamido)-2-(4-(dimethylamino)piperidin-1-yl)phenyl)acrylamide (YJZ6131)

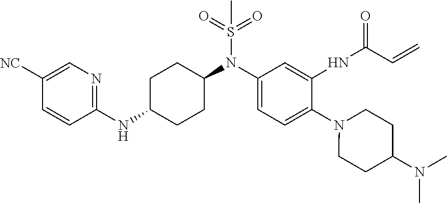 [SMILES: C=CC(=O)Nc1cc(N([C@H]2CC[C@H](Nc3ccc(C#N)cn3)CC2)S(C)(=O)=O)ccc1N1CCC(N(C)C)CC1]

The synthetic method was similar to the method as shown in Example 1.

¹H NMR (400 MHz, Chloroform-d) δ 8.51 (s, 1H), 8.40 (d, J=2.3 Hz, 1H), 8.29 (d, J=2.3 Hz, 1H), 7.50 (dd, J=8.8, 2.2 Hz, 1H), 7.16 (d, J=8.4 Hz, 1H), 7.04 (dd, J=8.4, 2.5 Hz, 1H), 6.51-6.46 (m, 2H), 6.34 (d, J=8.8 Hz, 1H), 5.87 (dd, J=7.1, 4.3 Hz, 1H), 4.99 (d, J=7.7 Hz, 1H), 4.22-4.10 (m, 1H), 3.61-3.50 (m, 1H), 3.24 (d, J=12.4 Hz, 2H), 3.03 (s, 3H), 2.94 (d, J=4.7 Hz, 1H), 2.90 (d, J=4.3 Hz, 6H), 2.83 (t, J=11.9 Hz, 2H), 2.43-2.27 (m, 4H), 2.14 (d, J=12.1 Hz, 2H), 2.07 (d, J=12.3 Hz, 2H), 1.43 (t, J=7.3 Hz, 4H).

HRMS (ESI) calcd for C₂₉H₃₉N₇O₃S [M+H]⁺, 566.2908; found, 566.2901.

### Example 29: N-(5-((N-((1r,4r)-4-((5-cyanopyridin-2-yl)amino)cyclohexyl)-1-phenylmethyl)sulfonamido)-2-(4-(dimethylamino)piperidin-1-yl)phenyl)acrylamide (YJZ6136)

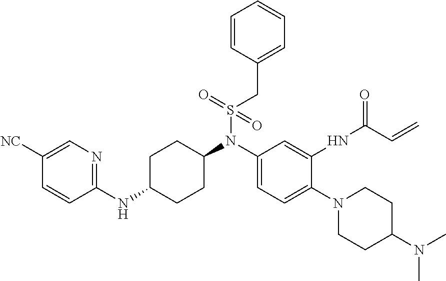 [SMILES: C=CC(=O)Nc1cc(N([C@H]2CC[C@H](Nc3ccc(C#N)cn3)CC2)S(=O)(=O)Cc2ccccc2)ccc1N1CCC(N(C)C)CC1]

The synthetic method was similar to the method as shown in Example 1.

¹H NMR (400 MHz, DMSO-d₆) δ 9.09 (s, 1H), 8.33 (d, J=2.3 Hz, 1H), 7.80 (s, 1H), 7.63 (dd, J=8.9, 2.4 Hz, 1H), 7.59 (d, J=7.3 Hz, 1H), 7.30-7.09 (m, 5H), 6.97 (d, J=8.4 Hz, 1H), 6.83 (d, J=8.4 Hz, 1H), 6.67 (dd, J=16.9, 10.2 Hz, 1H), 6.51 (d, J=8.9 Hz, 1H), 6.28 (dd, J=16.9, 1.9 Hz, 1H), 5.79 (dd, J=10.4, 2.0 Hz, 1H), 4.58 (s, 1H), 3.69-3.51 (m, 1H), 3.45-3.38 (m, 2H), 3.02 (d, J=10.2 Hz, 2H), 2.90 (s, 1H), 2.57 (s, 8H), 1.97 (p, J=10.4, 8.3 Hz, 6H), 1.89-1.75 (m, 2H), 1.52-1.19 (m, 4H).

HRMS (ESI) calcd for C₃₅H₄₃N₇O₃S [M+H]⁺, 509.0701; found, 509.0698.

### Example 30: N-(5-(N-((1r,4r)-4-((5-cyanopyridin-2-yl)amino)cyclohexyl)acetamido)-2-(4-(dimethylamino)piperidin-1-yl)phenyl)acrylamide (YJZ6115)

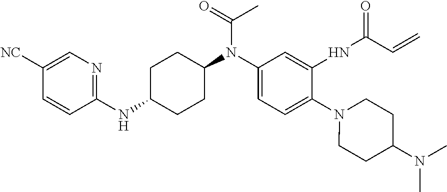 [SMILES: C=CC(=O)Nc1cc(N(C(C)=O)[C@H]2CC[C@H](Nc3ccc(C#N)cn3)CC2)ccc1N1CCC(N(C)C)CC1]

The synthetic method was similar to the method as shown in Example 1.

¹H NMR (400 MHz, DMSO-d₆) δ 9.23 (s, 1H), 8.39-8.28 (m, 1H), 7.84 (d, J=2.3 Hz, 1H), 7.66-7.56 (m, 2H), 7.21 (d, J=8.4 Hz, 1H), 6.96 (dd, J=8.3, 2.5 Hz, 1H), 6.88-6.71 (m, 1H), 6.50 (d, J=8.9 Hz, 1H), 6.29 (dd, J=17.0, 1.9 Hz, 1H), 5.81 (dd, J=10.1, 1.9 Hz, 1H), 4.44 (s, 1H), 3.49 (d, J=16.7 Hz, 1H), 3.34-3.24 (m, 1H), 3.18 (d, J=11.4 Hz, 2H), 2.75 (s, 8H), 2.11 (d, J=11.3 Hz, 2H), 2.05-1.98 (m, 2H), 1.94 (t, J=9.8 Hz, 2H), 1.77 (d, J=11.9 Hz, 2H), 1.65 (s, 3H), 1.40-1.27 (m, 2H), 1.20 (t, J=7.3 Hz, 2H).

HRMS (ESI) calcd for C₃₀H₃₉N₇O₂ [M+H]⁺, 530.3238; found, 530.3228.

### Example 31: N-(5-(3-benzyl-1-((1r,4S)-4-((5-cyanopyridin-2-yl)amino)cyclohexyl)ureido)-2-((S)-3,4-dimethylpiperazin-1-yl)-3-fluorophenyl)acrylamide (YJZ8087)

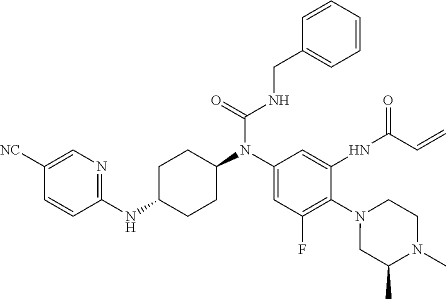 [SMILES: C=CC(=O)Nc1cc(N(C(=O)NCc2ccccc2)[C@H]2CC[C@H](Nc3ccc(C#N)cn3)CC2)cc(F)c1N1CCN(C)[C@@H](C)C1]

The synthetic method was similar to the method as shown in Example 1.

¹H NMR (400 MHz, DMSO-d₆) δ 9.07-8.99 (m, 2H), 8.33 (d, J=2.3 Hz, 1H), 7.98-7.87 (m, 1H), 7.63 (dd, J=8.9, 2.4 Hz, 1H), 7.56 (d, J=7.5 Hz, 1H), 7.23 (td, J=8.0, 6.1 Hz, 1H), 7.06 (d, J=8.4 Hz, 1H), 7.03-6.93 (m, 2H), 6.76-6.59 (m, 3H), 6.50 (d, J=8.9 Hz, 1H), 6.26 (dd, J=17.0, 1.9 Hz, 1H), 5.80 (dd, J=10.2, 1.9 Hz, 1H), 4.38 (t, J=3.6 Hz, 1H), 4.10 (d, J=6.0 Hz, 2H), 3.55 (s, 1H), 2.96-2.73 (m, 4H), 2.49-2.42 (m, 1H), 2.40-2.31 (m, 2H), 2.25 (s, 3H), 1.98 (d, J=11.4 Hz, 2H), 1.89-1.78 (m, 2H), 1.43-1.22 (m, 4H), 1.01 (d, J=6.4 Hz, 3H).

### Example 32: N-(5-(3-benzyl-1-((1r,4S)-4-((5-cyanopyridin-2-yl)amino)cyclohexyl)ureido)-2-((S)-3,4-dimethylpiperazin-1-yl)pyridin-3-yl)acrylamide (YJZ9171)

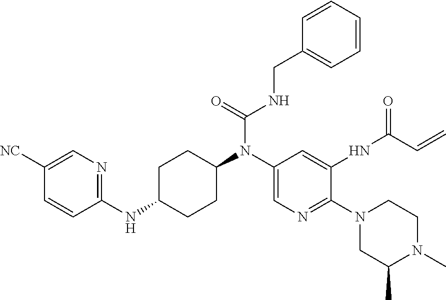 [SMILES: C=CC(=O)Nc1cc(N(C(=O)NCc2ccccc2)[C@H]2CC[C@H](Nc3ccc(C#N)cn3)CC2)cnc1N1CCN(C)[C@@H](C)C1]

The synthetic method was similar to the method as shown in Example 1.

¹H NMR (400 MHz, DMSO-d₆) δ 9.26 (s, 1H), 8.32 (d, J=2.3 Hz, 1H), 7.93 (s, 1H), 7.87 (d, J=2.4 Hz, 1H), 7.61 (dd, J=8.9, 2.4 Hz, 1H), 7.49 (d, J=7.6 Hz, 1H), 7.31-7.24 (m, 2H), 7.22-7.14 (m, 3H), 6.69 (dd, J=17.0, 10.2 Hz, 1H), 6.48 (d, J=8.9 Hz, 1H), 6.29 (dd, J=17.0, 2.0 Hz, 1H), 6.22 (t, J=6.0 Hz, 1H), 5.80 (dd, J=10.1, 1.9 Hz, 1H), 4.27 (tt, J=12, 3.6 Hz, 1H), 4.17 (d, J=5.9 Hz, 2H), 3.52 (s, 1H), 3.41-3.35 (m, 1H), 3.32-3.27 (s, 1H), 2.95 (td, J=11.9, 2.6 Hz, 1H), 2.76 (d, J=11.6 Hz, 1H), 2.57 (dd, J=12.2, 10.0 Hz, 1H), 2.45-2.37 (m, 1H), 2.33 (t, J=4.2 Hz, 1H), 2.23 (s, 3H), 1.93 (d, J=12.2 Hz, 2H), 1.79 (d, J=11.8 Hz, 2H), 1.33 (q, J=12.4 Hz, 2H), 1.11 (q, J=12.5 Hz, 2H), 1.00 (d, J=6.2 Hz, 3H).

### Example 33: N-(3-(3-benzyl-1-((1r,4r)-4-((5-cyanopyridin-2-yl)amino)cyclohexyl)ureido)-5-bromophenyl)acrylamide (YJZ8053)

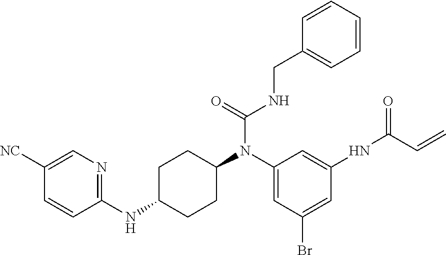 [SMILES: C=CC(=O)Nc1cc(Br)cc(N(C(=O)NCc2ccccc2)[C@H]2CC[C@H](Nc3ccc(C#N)cn3)CC2)c1]

The synthetic method was similar to the method as shown in Example 1.

¹H NMR (400 MHz, DMSO-d₆) δ 10.41 (s, 1H), 8.35-8.28 (m, 1H), 8.10 (t, J=1.9 Hz, 1H), 7.61 (dd, J=9.0, 2.4 Hz, 1H), 7.48 (d, J=7.6 Hz, 1H), 7.42 (t, J=1.9 Hz, 1H), 7.32-7.24 (m, 2H), 7.23-7.15 (m, 3H), 7.08 (t, J=1.8 Hz, 1H), 6.48 (d, J=8.9 Hz, 1H), 6.41 (dd, J=17.0, 9.9 Hz, 1H), 6.31 (dd, J=16.9, 2.2 Hz, 1H), 6.21 (t, J=6.0 Hz, 1H), 5.83 (dd, J=9.9, 2.2 Hz, 1H), 4.25 (d, J=12.0 Hz, 1H), 4.16 (d, J=5.9 Hz, 2H), 3.54 (s, 1H), 1.94 (d, J=11.5 Hz, 2H), 1.80 (d, J=11.8 Hz, 2H), 1.31 (t, J=12.2 Hz, 2H), 1.26-1.11 (m, 2H).

HRMS (ESI) calcd for C₂₉H₂₉BrN₆O₂ [M+H]⁺, 573.1608; found, 573.1601.

### Example 34: N-(3-(3-benzyl-1-((1r,4r)-4-((5-cyanopyridin-2-yl)amino)cyclohexyl)ureido)-5-methoxyphenyl)acrylamide (YJZ8058)

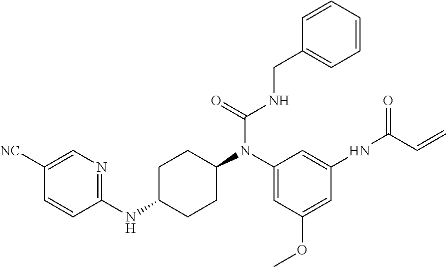 [SMILES: C=CC(=O)Nc1cc(OC)cc(N(C(=O)NCc2ccccc2)[C@H]2CC[C@H](Nc3ccc(C#N)cn3)CC2)c1]

The synthetic method was similar to the method as shown in Example 1.

¹H NMR (400 MHz, DMSO-d₆) δ 10.25 (s, 1H), 8.31 (dd, J=2.3, 0.7 Hz, 1H), 7.61 (dd, J=8.9, 2.4 Hz, 1H), 7.48 (q, J=3.8, 2.1 Hz, 2H), 7.31-7.24 (m, 2H), 7.23-7.15 (m, 3H), 7.08 (t, J=1.8 Hz, 1H), 6.51-6.37 (m, 3H), 6.28 (dd, J=17.0, 2.1 Hz, 1H), 5.93 (t, J=6.1 Hz, 1H), 5.82-5.75 (m, 1H), 4.25 (d, J=11.6 Hz, 1H), 4.16 (d, J=6.0 Hz, 2H), 3.76 (s, 3H), 3.54 (s, 1H), 1.98-1.88 (m, 2H), 1.80 (d, J=11.7 Hz, 2H), 1.32 (d, J=12.3 Hz, 2H), 1.27-1.19 (m, 2H).

HRMS (ESI) calcd for C₃₀H₃₂N₆O₃ [M+H]⁺, 525.2609; found, 525.2590.

HPLC purity=96.44%, Rt 10.63 min.

### Example 35: N-(3-(3-benzyl-1-(((1r,4S)-4-((5-cyanopyridin-2-yl)amino)cyclohexyl)ureido)-5-((S)-3,4-dimethylpiperazin-1-yl)phenyl)acrylamide (YJZ8062)

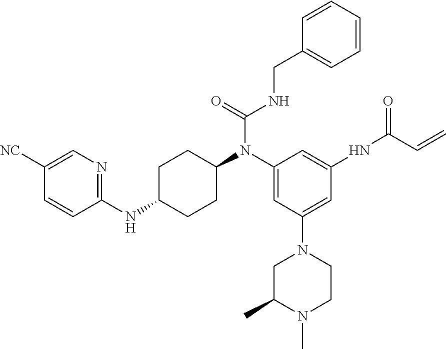 [SMILES: C=CC(=O)Nc1cc(N2CCN(C)[C@@H](C)C2)cc(N(C(=O)NCc2ccccc2)[C@H]2CC[C@H](Nc3ccc(C#N)cn3)CC2)c1]

The synthetic method was similar to the method as shown in Example 1.

¹H NMR (400 MHz, DMSO-d₆) δ 10.12 (s, 1H), 8.31 (d, J=2.3 Hz, 1H), 7.61 (dd, J=9.0, 2.3 Hz, 1H), 7.48 (d, J=7.6 Hz, 1H), 7.37 (s, 1H), 7.26 (dd, J=8.6, 6.2 Hz, 2H), 7.19 (d, J=7.1 Hz, 3H), 6.97 (s, 1H), 6.52-6.43 (m, 2H), 6.43-6.36 (m, 1H), 6.26 (dd, J=16.9, 2.1 Hz, 1H), 5.82-5.72 (m, 2H), 4.25 (t, J=11.9 Hz, 1H), 4.17 (d, J=6.0 Hz, 2H), 3.47 (t, J=13.0 Hz, 3H), 2.87-2.71 (m, 2H), 2.40 (t, J=10.9 Hz, 2H), 2.22 (s, 3H), 2.12 (t, J=6.4 Hz, 1H), 1.94 (d, J=11.8 Hz, 2H), 1.80 (d, J=11.7 Hz, 2H), 1.25 (q, J=12.8 Hz, 4H), 1.06 (d, J=6.1 Hz, 3H).

HRMS (ESI) calcd for C₃₅H₄₂N₈O₂ [M+H]⁺, 607.3503; found, 607.3477.

### Example 36: 3-benzyl-1-((1r,4r)-4-((5-cyanopyridin-2-yl)amino)cyclohexyl)-1-(4-(piperazin-1-yl)phenyl)urea (YJZ7037)

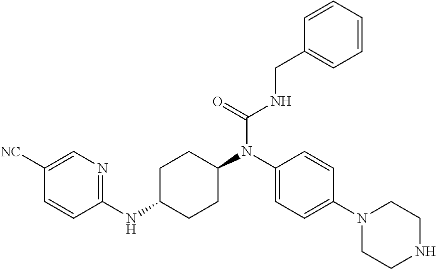 [SMILES: N#Cc1ccc(N[C@H]2CC[C@H](N(C(=O)NCc3ccccc3)c3ccc(N4CCNCC4)cc3)CC2)nc1]

The synthetic method was similar to the method as shown in Example 1.

¹H NMR (600 MHz, DMSO-d₆) δ 8.30 (d, J=2.3 Hz, 1H), 7.60 (d, J=6.7 Hz, 1H), 7.53 (s, 1H), 7.27 (t, J=7.5 Hz, 2H), 7.17 (dd, J=12.7, 7.3 Hz, 3H), 7.03-6.93 (m, 4H), 6.49 (d, J=8.9 Hz, 1H), 5.58 (t, J=6.2 Hz, 1H), 4.26 (tt, J=12.0, 3.0 Hz, 1H), 4.15 (d, J=6.0 Hz, 2H), 3.51 (s, 1H), 3.10 (t, J=5.0 Hz, 4H), 2.83 (t, J=5.0 Hz, 4H), 1.91 (d, J=12.2 Hz, 2H), 1.77 (d, J=10.5 Hz, 2H), 1.31 (q, J=13.8, 12.9 Hz, 2H), 1.24 (s, 1H), 1.10 (q, J=11.4 Hz, 2H). ¹³C NMR (151 MHz, DMSO-d₆) δ 159.71, 157.27, 153.55, 151.202, 141.80, 131.92, 128.47 (5 C), 127.14 (4 C), 126.68, 119.59, 115.74, 94.41, 53.36, 49.16 (2 C), 46.07 (2 C), 43.93, 40.52, 31.77 (2 C), 30.66 (2 C).

HRMS (ESI) for C₃₀H₃₅N₇O [M+H]⁺, calcd: 510.2976, found: 510.2956.

### Example 37: N1-(3-acrylamido-4-((S)-3,4-dimethylpiperazin-1-yl)phenyl)-N1-((1r,4S)-4-((5-cyanopyridin-2-yl)amino)cyclohexyl)-N2-(3,4-difluorobenzyl)oxalamide (YJZ8101)

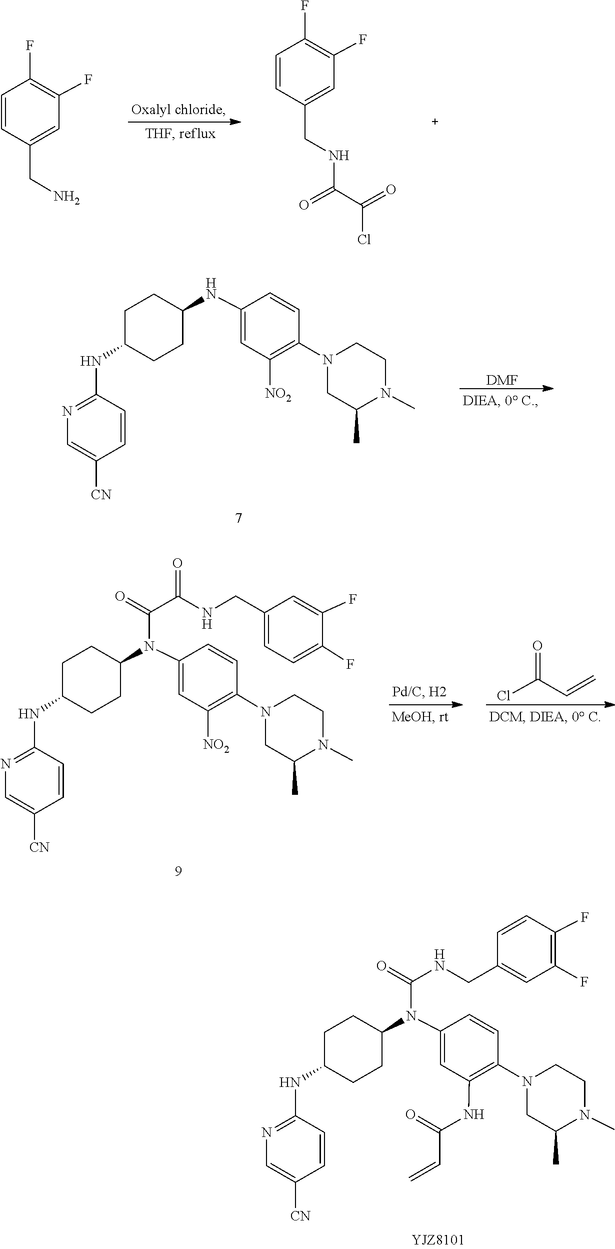

### Step 1: N1-((1r,4S)-4-((5-cyanopyridin-2-yl)amino)cyclohexyl)-N2-(3,4-difluorobenzyl)-N1-(4-((S)-3,4-dimethylpiperazin-1-yl)-3-nitrophenyl)oxalamide

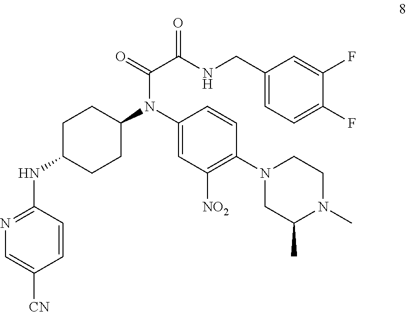 [SMILES: C[C@H]1CN(c2ccc(N(C(=O)C(=O)NCc3ccc(F)c(F)c3)[C@H]3CC[C@H](Nc4ccc(C#N)cn4)CC3)cc2[N+](=O)[O-])CCN1C]

(3,4-difluorophenyl)methylamine (239 mg, 1.67 mol) and oxalyl chloride (317.8 mg, 2.5 mol) were dissolved in 20 ml of anhydrous THF. The solution was refluxed under the protection of argon at 75° C. for 3 h, THF and excess oxalyl chloride were spin-dried, and the crude product was directly used in the next step reaction without purification. Compound 7 (250 mg, 0.56) (the synthetic method of the compound 7 was as shown in Example 1; step 3) and DIEA (72 mg, 0.56 mol) were dissolved in 10 ml of anhydrous DMF. The crude product after the reaction with oxalyl chloride was dissolved in 10 ml of THF, the THF solution was slowly added to the solution of the compound 7 in DMF at 0° C., and the mixture was warmed to room temperature for reaction for 30 min. After TLC detection showed that the reaction was complete, the solvent was spin-dried; the mixture was extracted with ethyl acetate and saturated sodium bicarbonate; and the organic phase was collected, concentrated, and subjected to column chromatography to provide 270 mg (yield: 75%).

¹H NMR (400 MHz, DMSO-d₆) δ 9.22 (t, J=6.1 Hz, 1H), 8.32-8.29 (m, 1H), 7.72 (s, 1H), 7.63 (t, J=8.7 Hz, 2H), 7.44-7.38 (m, 2H), 7.20 (dt, J=10.8, 8.5 Hz, 2H), 6.94 (ddd, J=11.7, 7.9, 2.2 Hz, 1H), 6.71 (d, J=6.6 Hz, 1H), 6.52 (d, J=8.9 Hz, 1H), 4.40-4.29 (m, 1H), 4.08 (d, J=5.9 Hz, 2H), 3.59 (dt, J=14.1, 6.5 Hz, 1H), 3.19-2.90 (m, 5H), 2.79 (s, 2H), 2.24 (s, 5H), 1.95 (q, J=9.0, 8.5 Hz, 3H), 1.84 (d, J=13.2 Hz, 2H), 1.42-1.32 (m, 3H), 1.32-1.18 (m, 10H), 1.05 (dd, J=13.6, 6.6 Hz, 4H).

### Step 2: N1-(3-acrylamido-4-((S)-3,4-dimethylpiperazin-1-yl)phenyl)-N1-((1r,4S)-4-((5-cyanopyridin-2-yl)amino)cyclohexyl)-N2-(3,4-difluorobenzyl)oxalamide (YJZ8101)

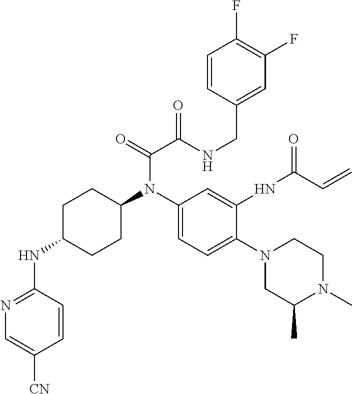 [SMILES: C=CC(=O)Nc1cc(N(C(=O)C(=O)NCc2ccc(F)c(F)c2)[C@H]2CC[C@H](Nc3ccc(C#N)cn3)CC2)ccc1N1CCN(C)[C@@H](C)C1]

The synthetic method was similar to the method as shown in step 5 of Example 1.

¹H NMR (400 MHz, DMSO-d₆) δ 9.05-8.98 (m, 2H), 8.32 (d, J=2.3 Hz, 1H), 7.92 (s, 1H), 7.62 (dd, J=8.8, 2.3 Hz, 1H), 7.51 (d, J=7.4 Hz, 1H), 7.22 (dt, J=10.8, 8.4 Hz, 1H), 7.03 (d, J=8.4 Hz, 1H), 6.98-6.90 (m, 2H), 6.69-6.59 (m, 2H), 6.49 (d, J=8.9 Hz, 1H), 6.26 (dd, J=16.9, 1.9 Hz, 1H), 5.83-5.76 (m, 1H), 4.37 (d, J=6.2 Hz, 2H), 4.06 (d, J=6.1 Hz, 2H), 3.55 (s, 1H), 2.84 (d, J=46.3 Hz, 4H), 2.45 (d, J=11.2 Hz, 1H), 2.39 (d, J=20.5 Hz, 2H), 2.25 (s, 3H), 1.97 (d, J=11.6 Hz, 2H), 1.86 (s, 2H), 1.35 (t, J=12.0 Hz, 3H), 1.24 (s, 2H), 1.00 (t, J=6.4 Hz, 3H).

HRMS (ESI) calcd for C₃₆H₄₀F₂N₈O₃ [M+H]⁺; found, 509.0698.

### Example 38: N1-(3-acrylamido-4-((S)-3,4-dimethylpiperazin-1-yl)phenyl)-N1-((1r,4S)-4-((5-cyanopyridin-2-yl)amino)cyclohexyl)-N2-(3-fluorobenzyl)oxalamide (YJZ8087)

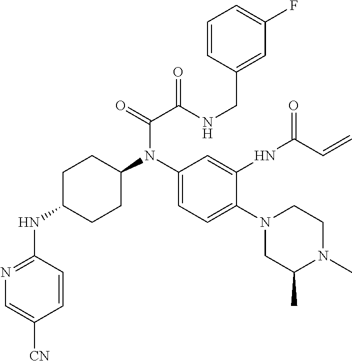 [SMILES: C=CC(=O)Nc1cc(N(C(=O)C(=O)NCc2cccc(F)c2)[C@H]2CC[C@H](Nc3ccc(C#N)cn3)CC2)ccc1N1CCN(C)[C@@H](C)C1]

The synthetic method was similar to the method as shown in Example 37.

¹H NMR (400 MHz, DMSO-d₆) δ 9.07-8.99 (m, 2H), 8.33 (d, J=2.3 Hz, 1H), 7.98-7.87 (m, 1H), 7.63 (dd, J=8.9, 2.4 Hz, 1H), 7.56 (d, J=7.5 Hz, 1H), 7.23 (td, J=8.0, 6.1 Hz, 1H), 7.06 (d, J=8.4 Hz, 1H), 7.03-6.93 (m, 2H), 6.76-6.59 (m, 3H), 6.50 (d, J=8.9 Hz, 1H), 6.26 (dd, J=17.0, 1.9 Hz, 1H), 5.80 (dd, J=10.2, 1.9 Hz, 1H), 4.38 (t, J=3.6 Hz, 1H), 4.10 (d, J=6.0 Hz, 2H), 3.55 (s, 1H), 2.96-2.73 (m, 4H), 2.48-2.30 (m, 3H), 2.25 (s, 3H), 1.98 (d, J=11.4 Hz, 2H), 1.89-1.78 (m, 2H), 1.43-1.22 (m, 4H), 1.01 (d, J=6.4 Hz, 3H).

HRMS (ESI) calcd for C₃₆H₄₁FN₈O₃[M+H]⁺, 653.3358; found, 653.3340.

### Example 39: N1-(3-acrylamido-4-((S)-3,4-dimethylpiperazin-1-yl)phenyl)-N1-((1r,4S)-4-((5-cyanopyridin-2-yl)amino)cyclohexyl)-N2-(4-fluorobenzyl)oxalamide (YJZ8093)

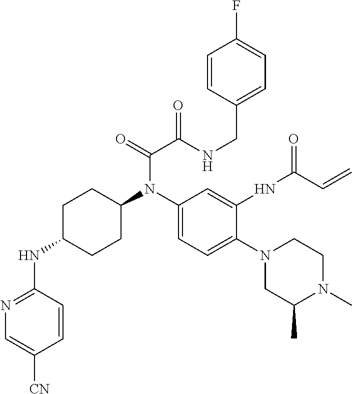 [SMILES: C=CC(=O)Nc1cc(N(C(=O)C(=O)NCc2ccc(F)cc2)[C@H]2CC[C@H](Nc3ccc(C#N)cn3)CC2)ccc1N1CCN(C)[C@@H](C)C1]

The synthetic method was similar to the method as shown in Example 37.

¹H NMR (400 MHz, DMSO-d₆) δ 9.08 (d, J=11.4 Hz, 1H), 8.96 (t, J=6.1 Hz, 1H), 8.33 (d, J=2.3 Hz, 1H), 7.90 (s, 1H), 7.62 (dd, J=8.9, 2.4 Hz, 1H), 7.52 (d, J=7.6 Hz, 1H), 7.00 (td, J=8.6, 6.1 Hz, 2H), 6.92 (dd, J=8.4, 2.5 Hz, 1H), 6.84 (dd, J=8.5, 5.6 Hz, 2H), 6.68 (dd, J=17.0, 10.2 Hz, 1H), 6.50 (d, J=8.9 Hz, 1H), 6.27 (dt, J=17.8, 4.1 Hz, 1H), 5.80 (dd, J=10.0, 2.0 Hz, 1H), 4.45-4.31 (m, 1H), 4.05 (d, J=6.2 Hz, 2H), 3.55 (s, 1H), 2.98-2.70 (m, 4H), 2.47-4.46 (m, 1H) 2.38 (s, 2H), 2.26 (s, 3H), 1.98 (d, J=11.7 Hz, 2H), 1.88-1.78 (m, 2H), 1.45-1.30 (m, 2H), 1.24 (s, 2H), 1.01 (d, J=5.7 Hz, 3H).

HRMS (ESI) calcd for C₃₆H₄₁FN₈O₃[M+H]⁺, 653.3358; found, 653.3337.

### Example 40: N1-(3-acrylamido-4-((S)-3,4-dimethylpiperazin-1-yl)phenyl)-N1-((1r,4S)-4-((5-cyanopyridin-2-yl)amino)cyclohexyl)-N2-(2,3-difluorobenzyl)oxalamide (YJZ8107)

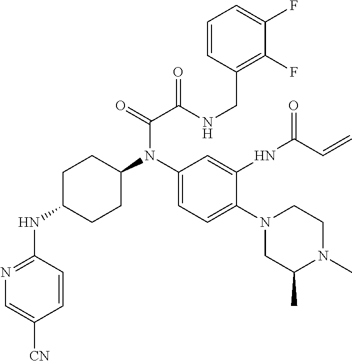 [SMILES: C=CC(=O)Nc1cc(N(C(=O)C(=O)NCc2cccc(F)c2F)[C@H]2CC[C@H](Nc3ccc(C#N)cn3)CC2)ccc1N1CCN(C)[C@@H](C)C1]

The synthetic method was similar to the method as shown in Example 37.

¹H NMR (400 MHz, DMSO-d₆) δ 9.05 (dd, J=11.4, 5.3 Hz, 2H), 8.35-8.32 (m, 1H), 7.94-7.86 (m, 1H), 7.63 (dd, J=8.9, 2.4 Hz, 1H), 7.55 (d, J=7.5 Hz, 1H), 7.31-7.24 (m, 1H), 7.03 (d, J=8.5 Hz, 1H), 7.01-6.96 (m, 1H), 6.93 (dd, J=8.4, 2.5 Hz, 1H), 6.64 (d, J=10.4 Hz, 1H), 6.50 (d, J=8.9 Hz, 2H), 6.27 (dd, J=17.0, 1.9 Hz, 1H), 5.84-5.76 (m, 1H), 4.43-4.30 (m, 1H), 4.15 (d, J=5.8 Hz, 2H), 3.53 (d, J=15.5 Hz, 1H), 2.98-2.70 (m, 4H), 2.49-2.44 (m, 1H), 2.37 (s, 2H), 2.25 (d, J=3.1 Hz, 3H), 1.96 (s, 2H), 1.84 (d, J=11.1 Hz, 2H), 1.42-1.21 (m, 4H), 1.01 (dd, J=5.8, 2.8 Hz, 3H).

HRMS (ESI) calcd for C₃₆H₄₀F₂N₈O₃ [M+H]⁺, 671.3264; found, 671.3246.

### Example 41: N1-(3-acrylamido-4-((S)-3,4-dimethylpiperazin-1-yl)phenyl)-N1-((1r,4S)-4-((5-cyanopyridin-2-yl)amino)cyclohexyl)-N2-(3,5-difluorobenzyl)oxalamide (YJZ8102)

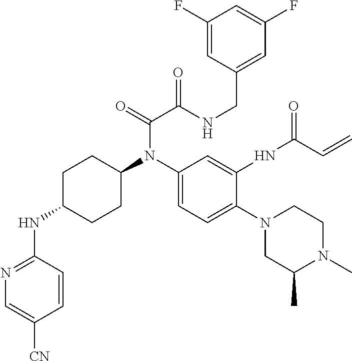 [SMILES: C=CC(=O)Nc1cc(N(C(=O)C(=O)NCc2cc(F)cc(F)c2)[C@H]2CC[C@H](Nc3ccc(C#N)cn3)CC2)ccc1N1CCN(C)[C@@H](C)C1]

The synthetic method was similar to the method as shown in Example 37.

¹H NMR (400 MHz, DMSO-d₆) δ 9.05 (t, J=6.1 Hz, 1H), 8.95 (s, 1H), 8.33 (d, J=2.3 Hz, 1H), 7.95 (s, 1H), 7.62 (dd, J=8.8, 2.4 Hz, 1H), 7.52 (d, J=7.5 Hz, 1H), 7.11-7.00 (m, 2H), 6.96 (dd, J=8.3, 2.5 Hz, 1H), 6.62 (d, J=7.5 Hz, 3H), 6.50 (d, J=8.9 Hz, 1H), 6.25 (dd, J=16.9, 1.9 Hz, 1H), 5.84-5.76 (m, 1H), 4.37 (s, 1H), 4.11 (d, J=6.0 Hz, 2H), 3.55 (s, 1H), 2.93-2.72 (m, 4H), 2.39 (d, J=44.0 Hz, 3H), 2.25 (s, 3H), 1.96 (s, 3H), 1.84 (d, J=10.8 Hz, 2H), 1.35 (t, J=12.0 Hz, 2H), 1.24 (s, 2H), 1.00 (d, J=5.4 Hz, 3H).

HRMS (ESI) calcd for C₃₆H₄₀F₂N₈O₃ [M+H]⁺, 671.3264; found, 671.3242.

### Example 42: N1-(3-acrylamido-4-((S)-3,4-dimethylpiperazin-1-yl)phenyl)-N1-((1r,4R)-4-((5-cyanopyridin-2-yl)amino)cyclohexyl)-N2-((R)-1-phenylethyl)oxalamide (YJZ8105)

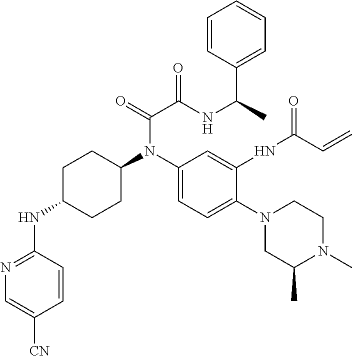 [SMILES: C=CC(=O)Nc1cc(N(C(=O)C(=O)N[C@H](C)c2ccccc2)[C@H]2CC[C@H](Nc3ccc(C#N)cn3)CC2)ccc1N1CCN(C)[C@@H](C)C1]

The synthetic method was similar to the method as shown in Example 37.

¹H NMR (400 MHz, DMSO-d₆) δ 9.04 (s, 1H), 8.82 (d, J=8.1 Hz, 1H), 8.33 (d, J=2.4 Hz, 1H), 7.93 (s, 1H), 7.62 (dd, J=8.9, 2.5 Hz, 1H), 7.52 (d, J=7.5 Hz, 1H), 7.22 (t, J=7.4 Hz, 2H), 7.17 (d, J=7.0 Hz, 1H), 7.03 (d, J=8.0 Hz, 3H), 6.95 (dd, J=8.4, 2.5 Hz, 1H), 6.73-6.61 (m, 1H), 6.50 (d, J=8.9 Hz, 1H), 6.32-6.22 (m, 1H), 5.79 (d, J=10.3 Hz, 1H), 4.62 (t, J=7.3 Hz, 1H), 4.36 (s, 1H), 3.54 (s, 3H), 2.79 (d, J=11.1 Hz, 4H), 2.47 (m, 1H), 2.36 (s, 2H), 2.25 (s, 3H), 1.98 (s, 2H), 1.85 (s, 2H), 1.35 (t, J=12.0 Hz, 2H), 1.24 (s, 2H), 1.11 (d, J=7.0 Hz, 3H), 1.00 (dd, J=5.9, 2.9 Hz, 3H).

HRMS (ESI) calcd for C₃₇H₄₄N₈O₃ [M+H]⁺, 671.3264; found, 671.3261.

### Example 43: N1-(3-acrylamido-4-((S)-3,4-dimethylpiperazin-1-yl)phenyl)-N1-((1r,4S)-4-((5-cyanopyridin-2-yl)amino)cyclohexyl)-N2-((S)-1-phenylethyl)oxalamide

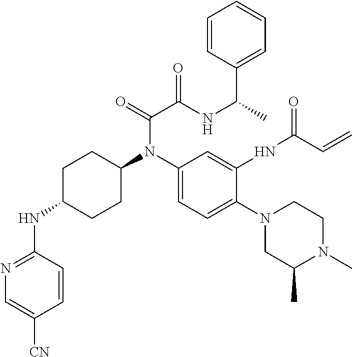 [SMILES: C=CC(=O)Nc1cc(N(C(=O)C(=O)N[C@@H](C)c2ccccc2)[C@H]2CC[C@H](Nc3ccc(C#N)cn3)CC2)ccc1N1CCN(C)[C@@H](C)C1]

The synthetic method was similar to the method as shown in Example 37.

¹H NMR (400 MHz, DMSO-d₆) δ 9.07 (s, 1H), 8.86 (d, J=8.1 Hz, 1H), 8.33 (d, J=2.3 Hz, 1H), 7.92 (s, 1H), 7.63 (dd, J=8.8, 2.4 Hz, 1H), 7.56 (d, J=7.5 Hz, 1H), 7.40 (d, J=4.3 Hz, 1H), 7.23 (dd, J=9.5, 5.3 Hz, 2H), 7.17 (d, J=7.1 Hz, 1H), 7.07-6.98 (m, 2H), 6.95 (dd, J=8.4, 2.4 Hz, 1H), 6.67 (dt, J=17.6, 9.4 Hz, 1H), 6.50 (d, J=9.0 Hz, 1H), 6.33-6.23 (m, 1H), 5.79 (d, J=9.9 Hz, 1H), 4.66-4.57 (m, 1H), 4.37 (s, 1H), 3.64-3.52 (m, 1H), 2.96-2.70 (m, 4H), 2.48-2.42 (m, 1H), 2.40-2.30 (m, 2H), 2.25 (s, 3H), 2.04-1.94 (m, 2H), 1.85 (s, 2H), 1.42-1.25 (m, 4H), 1.12 (d, J=7.0 Hz, 3H), 1.01 (dd, J=6.9, 3.2 Hz, 4H).

HRMS (ESI) calcd for C₃₇H₄₄N₈O₃ [M+H]⁺, 649.3609; found, 649.3615.

### Example 44: N1-(3-acrylamido-4-((S)-3,4-dimethylpiperazin-1-yl)phenyl)-N1-((1r,4S)-4-((5-cyanopyridin-2-yl)amino)cyclohexyl)-N2-(cyclopropylmethyl)oxalamide (YJZ8103)

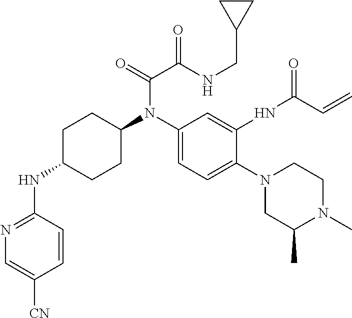 [SMILES: C=CC(=O)Nc1cc(N(C(=O)C(=O)NCC2CC2)[C@H]2CC[C@H](Nc3ccc(C#N)cn3)CC2)ccc1N1CCN(C)[C@@H](C)C1]

The synthetic method was similar to the method as shown in Example 37.

¹H NMR (400 MHz, Chloroform-d) δ 8.61 (s, 1H), 8.29 (dd, J=8.0, 2.3 Hz, 2H), 7.50 (dd, J=8.8, 2.3 Hz, 1H), 7.17 (dd, J=7.2, 5.0 Hz, 2H), 6.89 (dd, J=8.4, 2.5 Hz, 1H), 6.41 (dd, J=16.9, 1.3 Hz, 1H), 6.34-6.23 (m, 2H), 5.82 (dd, J=10.1, 1.3 Hz, 1H), 5.03 (d, J=7.8 Hz, 1H), 4.58 (s, 1H), 3.53 (d, J=22.4 Hz, 1H), 3.01-2.95 (m, 4H), 2.89 (dd, J=11.6, 2.8 Hz, 1H), 2.51-2.43 (m, 1H), 2.40 (s, 3H), 2.30 (s, 1H), 2.14 (d, J=11.0 Hz, 2H), 1.98 (s, 2H), 1.83 (s, 2H), 1.55-1.34 (m, 4H), 1.15 (d, J=6.2 Hz, 3H), 0.94-0.80 (m, 1H), 0.50-0.39 (m, 2H), 0.15-0.07 (m, 2H).

HRMS (ESI) calcd for C₃₃H₄₂N₈O₃ [M+H]⁺, 599.3453; found, 599.3435.

### Example 45: N1-(3-acrylamido-4-((S)-3,4-dimethylpiperazin-1-yl)phenyl)-N1-((1r,4S)-4-((5-cyanopyridin-2-yl)amino)cyclohexyl)-N2-cyclopropyloxalamide (YJZ8095)

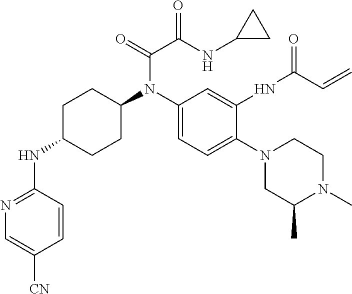 [SMILES: C=CC(=O)Nc1cc(N(C(=O)C(=O)NC2CC2)[C@H]2CC[C@H](Nc3ccc(C#N)cn3)CC2)ccc1N1CCN(C)[C@@H](C)C1]

The synthetic method was similar to the method as shown in Example 37.

¹H NMR (400 MHz, Chloroform-d) δ 8.61 (s, 1H), 8.30 (dd, J=2.3, 0.7 Hz, 1H), 8.25 (d, J=2.4 Hz, 1H), 7.51 (dd, J=8.8, 2.3 Hz, 1H), 7.20-7.12 (m, 2H), 6.89 (dd, J=8.4, 2.5 Hz, 1H), 6.42 (dd, J=16.9, 1.3 Hz, 1H), 6.34-6.24 (m, 2H), 5.83 (dd, J=10.1, 1.3 Hz, 1H), 5.08 (s, 1H), 4.55 (d, J=3.6 Hz, 1H), 3.51 (s, 1H), 3.03-2.94 (m, 2H), 2.93-2.87 (m, 1H), 2.55 (dq, J=7.2, 3.6 Hz, 1H), 2.52-2.44 (m, 1H), 2.41 (s, 3H), 2.33 (s, 1H), 2.14 (d, J=10.8 Hz, 2H), 2.06-1.88 (m, 4H), 1.42 (h, J=12.8, 10.3 Hz, 4H), 1.16 (d, J=6.2 Hz, 3H), 0.69 (dq, J=7.1, 2.2 Hz, 2H), 0.43 (s, 2H).

HRMS (ESI) calcd for C₃₂H₄₀N₈O₃[M+H]⁺, 585.3296; found, 585.3294.

### Example 46: N1-(3-acrylamido-4-((S)-3,4-dimethylpiperazin-1-yl)phenyl)-N1-((1r,4S)-4-((5-cyanopyridin-2-yl)amino)cyclohexyl)-N2-(2,2,2-trifluoroethyl)oxalamide (YJZ8104)

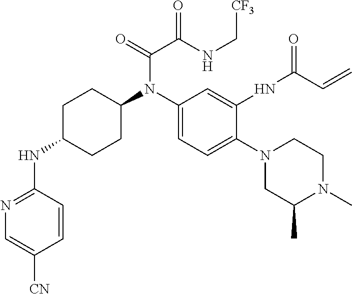 [SMILES: C=CC(=O)Nc1cc(N(C(=O)C(=O)NCC(F)(F)F)[C@H]2CC[C@H](Nc3ccc(C#N)cn3)CC2)ccc1N1CCN(C)[C@@H](C)C1]

The synthetic method was similar to the method as shown in Example 37.

¹H NMR (400 MHz, Chloroform-d) δ 8.62 (s, 1H), 8.30 (t, J=2.8 Hz, 2H), 7.63-7.44 (m, 2H), 7.17 (d, J=8.4 Hz, 1H), 6.88 (dd, J=8.4, 2.5 Hz, 1H), 6.43 (dd, J=16.8, 1.3 Hz, 1H), 6.35-6.24 (m, 2H), 5.84 (dd, J=10.1, 1.2 Hz, 1H), 5.03 (s, 1H), 4.58 (t, J=11.8 Hz, 1H), 3.82-3.70 (m, 2H), 3.51 (s, 1H), 3.03-2.93 (m, 2H), 2.92-2.85 (m, 1H), 2.53-2.44 (m, 1H), 2.41 (s, 3H), 2.33 (s, 1H), 2.16 (d, J=11.0 Hz, 2H), 1.79 (s, 4H), 1.43 (t, J=11.5 Hz, 4H), 1.16 (d, J=6.3 Hz, 3H).

HRMS (ESI) calcd for C₃₁H₃₇F₃N₈O₃ [M+H]⁺, 627.3013; found, 627.2994.

### Example 47: N-(5-(3-benzyl-1-((1r,4S)-4-((5-chloropyridin-2-yl)amino)cyclohexyl)ureido)-2-((S)-3,4-dimethylpiperazin-1-yl)phenyl)acrylamide(YJZ1070)

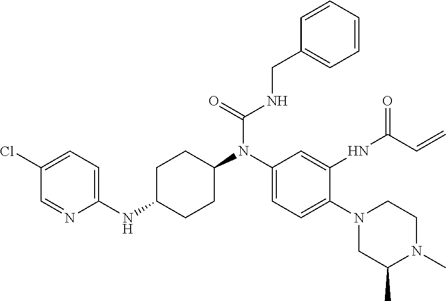 [SMILES: C=CC(=O)Nc1cc(N(C(=O)NCc2ccccc2)[C@H]2CC[C@H](Nc3ccc(Cl)cn3)CC2)ccc1N1CCN(C)[C@@H](C)C1]

The synthetic method was similar to the method as shown in Example 1.

¹H NMR (400 MHz, DMSO-d₆) δ 9.02 (s, 1H), 7.91 (s, 1H), 7.87 (d, J=2.8 Hz, 1H), 7.34 (dd, J=9.0, 2.8 Hz, 1H), 7.26 (q, J=11.1, 9.4 Hz, 3H), 7.19 (t, J=6.8 Hz, 4H), 6.91 (d, J=8.3 Hz, 1H), 6.70-6.57 (m, 2H), 6.42 (d, J=8.9 Hz, 1H), 6.27 (d, J=16.9 Hz, 1H), 5.86-5.69 (m, 2H), 4.27 (t, J=11.9 Hz, 1H), 4.17 (d, J=6.0 Hz, 2H), 3.01-2.72 (m, 4H), 2.49-2.41 (m, 2H), 2.40-2.31 (m, 1H), 2.25 (s, 3H), 1.93 (d, J=12.1 Hz, 2H), 1.77 (d, J=11.9 Hz, 2H), 1.26 (q, J=11.4 Hz, 2H), 1.16 (q, J=11.5 Hz, 2H), 1.00 (d, J=6.3 Hz, 3H).

### Example 48: N-(5-(3-(2-chlorobenzyl)-1-((1r,4S)-4-((5-cyanopyridin-2-yl)amino)cyclohexyl)ureido)-2-((S)-3,4-dimethylpiperazin-1-yl)phenyl)acrylamide (YJZ1060)

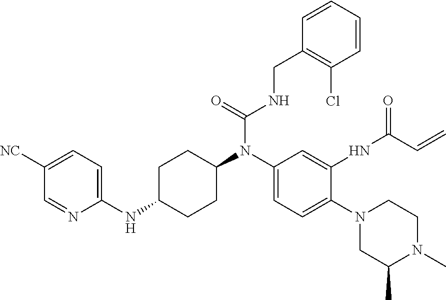 [SMILES: C=CC(=O)Nc1cc(N(C(=O)NCc2ccccc2Cl)[C@H]2CC[C@H](Nc3ccc(C#N)cn3)CC2)ccc1N1CCN(C)[C@@H](C)C1]

The synthetic method was similar to the method as shown in Example 1.

¹H NMR (400 MHz, DMSO-d₆) δ 9.04 (s, 1H), 8.31 (d, J=2.3 Hz, 1H), 7.97 (s, 1H), 7.60 (dd, J=8.8, 2.4 Hz, 1H), 7.49 (d, J=7.6 Hz, 1H), 7.39-7.34 (m, 1H), 7.34-7.29 (m, 1H), 7.28-7.20 (m, 3H), 6.98 (dd, J=8.4, 2.4 Hz, 1H), 6.67 (dd, J=16.9, 10.2 Hz, 1H), 6.47 (d, J=8.9 Hz, 1H), 6.28 (dd, J=16.9, 1.9 Hz, 1H), 5.87-5.77 (m, 2H), 4.28 (dt, J=7.5, 3.6 Hz, 1H), 4.23 (d, J=6.0 Hz, 2H), 3.51 (s, 1H), 2.97-2.76 (m, 4H), 2.49-2.42 (m, 2H), 2.40-2.32 (m, 1H), 2.25 (s, 3H), 1.93 (d, J=11.9 Hz, 2H), 1.80 (d, J=11.9 Hz, 2H), 1.32 (q, J=12.2 Hz, 2H), 1.23-1.10 (m, 2H), 1.01 (d, J=6.1 Hz, 3H).

### Example 49: N-(5-(1-((1r,4S)-4-((5-cyanopyridin-2-yl)amino)cyclohexyl)-3-(pyridin-3-ylmethyl)ureido)-2-((S)-3,4-dimethylpiperazin-1-yl)phenyl)acrylamide (YJZ1061)

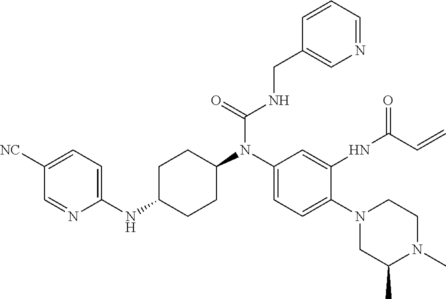 [SMILES: C=CC(=O)Nc1cc(N(C(=O)NCc2cccnc2)[C@H]2CC[C@H](Nc3ccc(C#N)cn3)CC2)ccc1N1CCN(C)[C@@H](C)C1]

The synthetic method was similar to the method as shown in Example 1.

¹H NMR (400 MHz, DMSO-d₆) δ 9.02 (s, 1H), 8.39 (t, J=2.2 Hz, 2H), 8.30 (d, J=2.4 Hz, 1H), 7.89 (s, 1H), 7.60 (ddt, J=8.1, 5.6, 2.2 Hz, 2H), 7.48 (d, J=7.6 Hz, 1H), 7.30 (dd, J=7.9, 4.8 Hz, 1H), 7.21 (d, J=8.3 Hz, 1H), 6.91 (dd, J=8.4, 2.6 Hz, 1H), 6.66 (dd, J=16.8, 10.2 Hz, 1H), 6.47 (d, J=8.9 Hz, 1H), 6.27 (dd, J=16.9, 1.9 Hz, 1H), 5.94 (t, J=6.1 Hz, 1H), 5.79 (dd, J=10.2, 1.9 Hz, 1H), 4.25 (t, J=11.8 Hz, 1H), 4.17 (d, J=5.9 Hz, 2H), 3.49 (s, 1H), 2.96-2.76 (m, 4H), 2.49-2.41 (m, 2H), 2.40-2.29 (m, 1H), 2.25 (s, 3H), 1.91 (d, J=12.2 Hz, 2H), 1.77 (d, J=11.6 Hz, 2H), 1.31 (q, J=12.7 Hz, 2H), 1.13 (q, J=13.5, 13.1 Hz, 2H), 1.01 (d, J=6.1 Hz, 3H).

### Example 50: N-(5-(1-((1r,4S)-4-((5-cyanopyridin-2-yl)amino)cyclohexyl)-3-(oxazol-4-ylmethyl)ureido)-2-((S)-3,4-dimethylpiperazin-1-yl)phenyl)acrylamide (YJZ1063)

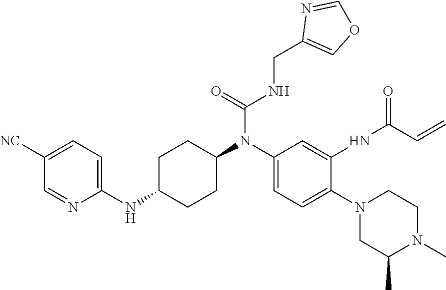 [SMILES: C=CC(=O)Nc1cc(N(C(=O)NCc2cocn2)[C@H]2CC[C@H](Nc3ccc(C#N)cn3)CC2)ccc1N1CCN(C)[C@@H](C)C1]

The synthetic method was similar to the method as shown in Example 1.

¹H NMR (400 MHz, DMSO-d₆) δ 9.04 (s, 1H), 8.31 (d, J=2.4 Hz, 1H), 8.23 (s, 1H), 7.85 (s, 1H), 7.75 (s, 1H), 7.60 (dd, J=8.9, 2.4 Hz, 1H), 7.49 (d, J=7.5 Hz, 1H), 7.20 (d, J=8.4 Hz, 1H), 6.92 (dd, J=8.4, 2.5 Hz, 1H), 6.65 (dd, J=16.9, 10.2 Hz, 1H), 6.47 (d, J=8.8 Hz, 1H), 6.26 (dd, J=16.9, 1.8 Hz, 1H), 5.85-5.72 (m, 1H), 5.56 (t, J=5.8 Hz, 1H), 4.26 (t, J=12.2 Hz, 1H), 4.17 (d, J=6.0 Hz, 2H), 3.48 (s, 1H), 2.99-2.75 (m, 4H), 2.49-2.40 (m, 2H), 2.40-2.31 (m, 1H), 2.25 (s, 3H), 1.92 (d, J=12.1 Hz, 2H), 1.79 (d, J=12.1 Hz, 2H), 1.31 (q, J=12.7 Hz, 2H), 1.13 (q, J=12.1, 11.6 Hz, 2H), 1.01 (d, J=6.1 Hz, 3H).

### Example 51: N-(5-(1-((1r,4S)-4-((5-cyanopyridin-2-yl)amino)cyclohexyl)-3-(2,2,2-trifluoroethyl)ureido)-2-((S)-3,4-dimethylpiperazin-1-yl)phenyl)acrylamide (YJZ1065)

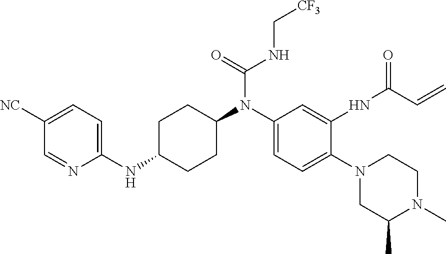 [SMILES: C=CC(=O)Nc1cc(N(C(=O)NCC(F)(F)F)[C@H]2CC[C@H](Nc3ccc(C#N)cn3)CC2)ccc1N1CCN(C)[C@@H](C)C1]

The synthetic method was similar to the method as shown in Example 1.

¹H NMR (400 MHz, DMSO-d₆) δ 9.02 (s, 1H), 8.31 (d, J=2.3 Hz, 1H), 7.91-7.79 (m, 1H), 7.61 (dd, J=8.8, 2.4 Hz, 1H), 7.49 (d, J=7.6 Hz, 1H), 7.22 (d, J=8.4 Hz, 1H), 6.87 (dd, J=8.3, 2.4 Hz, 1H), 6.66 (dd, J=17.0, 10.2 Hz, 1H), 6.47 (d, J=8.9 Hz, 1H), 6.26 (dd, J=17.0, 1.9 Hz, 1H), 5.83 (t, J=6.3 Hz, 1H), 5.79 (dd, J=10.2, 1.8 Hz, 1H), 4.26 (t, J=12.2 Hz, 1H), 3.79-3.66 (m, 2H), 3.50 (s, 1H), 2.97-2.76 (m, 4H), 2.49-2.43 (m, 2H), 2.40-2.31 (m, 1H), 2.25 (s, 3H), 1.92 (d, J=12.1 Hz, 2H), 1.83-1.71 (m, 2H), 1.32 (q, J=12.3 Hz, 2H), 1.16 (q, J=12.3 Hz, 2H), 1.01 (d, J=6.1 Hz, 3H).

### Example 52: N-(5-(3-benzyl-1-((1r,4S)-4-((5-(trifluoromethyl)pyridin-2-yl)amino)cyclohexyl)ureido)-2-((S)-3,4-dimethylpiperazin-1-yl)phenyl)acrylamide (YJZ1066)

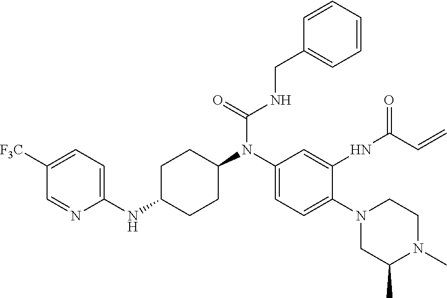 [SMILES: C=CC(=O)Nc1cc(N(C(=O)NCc2ccccc2)[C@H]2CC[C@H](Nc3ccc(C(F)(F)F)cn3)CC2)ccc1N1CCN(C)[C@@H](C)C1]

The synthetic method was similar to the method as shown in Example 1.

¹H NMR (400 MHz, DMSO-d₆) δ 9.02 (s, 1H), 8.21 (d, J=2.5 Hz, 1H), 7.92 (s, 1H), 7.55 (dd, J=8.9, 2.6 Hz, 1H), 7.31-7.24 (m, 2H), 7.24-7.13 (m, 5H), 6.92 (dd, J=8.3, 2.5 Hz, 1H), 6.66 (dd, J=16.9, 10.2 Hz, 1H), 6.50 (d, J=8.9 Hz, 1H), 6.27 (dd, J=16.9, 1.9 Hz, 1H), 5.85-5.72 (m, 2H), 4.27 (t, J=12.2 Hz, 1H), 4.17 (d, J=6.0 Hz, 2H), 3.49 (s, 1H), 2.98-2.75 (m, 4H), 2.49-2.41 (m, 2H), 2.41-2.31 (m, 1H), 2.25 (s, 3H), 1.94 (d, J=11.6 Hz, 2H), 1.85-1.72 (m, 2H), 1.30 (q, J=12.4 Hz, 2H), 1.17 (q, J=12.5 Hz, 2H) 1.01 (d, J=6.1 Hz, 3H).

### Example 53: N-(5-(3-benzyl-1-((1r,4S)-4-((5-cyanopyridin-2-yl)amino)cyclohexyl)ureido)-2-((S)-3,4-dimethylpiperazin-1-yl)phenyl)-2-fluoroacrylamide (YJZ1072)

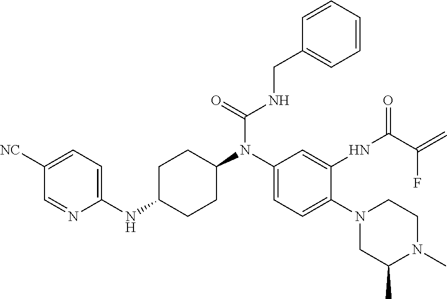 [SMILES: C=C(F)C(=O)Nc1cc(N(C(=O)NCc2ccccc2)[C@H]2CC[C@H](Nc3ccc(C#N)cn3)CC2)ccc1N1CCN(C)[C@@H](C)C1]

The synthetic method was similar to the method as shown in Example 1.

¹H NMR (400 MHz, DMSO-d₆) δ 9.42 (t, J=2.8 Hz, 1H), 8.30 (s, 1H), 8.08 (s, 1H), 7.60 (d, J=8.9 Hz, 1H), 7.49 (d, J=7.6 Hz, 1H), 7.34 (d, J=8.3 Hz, 1H), 7.30-7.23 (m, 2H), 7.22-7.13 (m, 3H), 6.97 (d, J=8.3 Hz, 1H), 6.47 (d, J=8.9 Hz, 1H), 5.90-5.80 (m, 1.48H), 5.71 (s, 0.52H), 5.53 (d, J=15.0 Hz 1H), 4.28 (t, J=12.2 Hz, 1H), 4.17 (d, J=5.9 Hz, 2H), 3.49 (s, 1H), 2.97-2.81 (m, 4H), 2.56-2.53 (m, 1H), 2.30 (t, J=10.6 Hz, 1H), 2.24 (s, 3H), 2.23-2.15 (m, 1H), 1.92 (d, J=12.1 Hz, 2H), 1.79 (d, J=12.1 Hz, 2H), 1.32 (q, J=12.4 Hz, 2H), 1.16 (q, J=12.5 Hz, 2H), 1.03 (d, J=6.2 Hz, 3H).

### Example 54: IC₅₀ Test of Compound YJZ5118 on CDK12, CDK13, and CDK9 Kinases

The inhibitory activities of the test compound on the kinases were evaluated by LANCE Ultra assay, which was used for detecting ATP-dependent phosphorylation of a ULight-4E-BP1 (Thr37/Thr46) substrate peptide (150 nM). In short, enzymatic reactions were performed in a reaction buffer (25 mM HEPES (pH 7.5), 10 mM MgCl₂, 0.01% BSA, 0.01% Tween 20, and 1 mM DTT). The assay was performed in a 384-well plate (6 μL). A final concentration of an ATP substrate was 10 M, and a final concentration of the ULight-4E-BP1 (Thr37/Thr46) substrate peptide was 150 nM. A final concentration of CDK9, CDK12, or CDK13 was 0.3, 30, or 30 nM, respectively. Pre-incubation of the compound and the enzyme was performed at room temperature for 60 min. The reaction was stopped by adding 10 mM EDTA and 0.15 nM Eu-labeled anti-phospho-eIF4E-binding protein 1 (Thr37/46) antibody in LANCE detection buffer after incubation at room temperature for 15, 60, or 90 min. Time-resolved fluorescence (excitation, 320 nm; emission donor, 615 nm; emission acceptor, 665 nm) was monitored using an EnVision spectrophotometer (PerkinElmer). Readings were computed as (acceptor count/donor count)×1000. IC50 values were derived by fitting a sigmoidal dose-response curve to a plot of assay readouts of inhibitor concentrations. All fittings were computed using program Prism 5.03 (GraphPad Software, San Diego, CA).

The kinase activity test result is as shown in Table 1.

TABLE 1
in vitro inhibitory activities of compounds
against CDK12, CDK13 and CDK9.

| | CDK12 | CDK13 | CDK9 |
|---|---|---|---|
| Compounds | IC₅₀(nM) | IC₅₀(nM) | IC₅₀(nM) |
| YJZ5118 | ** | ** | **** |
| YJZ4156 | **** | *** | # |
| YJZ5028 | ** | * | # |
| YJZ6048 | ** | * | # |
| YJZ5125 | *** | *** | # |
| YJZ6077 | *** | *** | # |
| YJZ6082 | **** | *** | # |
| YJZ5127 | *** | * | # |
| YJZ5114 | *** | *** | # |
| YJZ8027 | ** | * | # |
| YJZ6093 | ** | ** | # |
| YJZ5111 | ** | ** | # |
| YJZ6028 | *** | *** | # |
| YJZ6096 | ** | ** | # |
| YJZ1070 | ** | ** | # |
| YJZ1066 | ** | * | # |
| YJZ9170 | ** | ** | # |
| YJZ9176 | **** | *** | # |
| YJZ9179 | ** | * | # |
| YJZ9177 | ** | * | # |
| YJZ9166 | ** | ** | # |
| YJZ1060 | * | * | # |
| YJZ1061 | *** | *** | # |
| YJZ1033 | **** | *** | # |
| YJZ1063 | *** | *** | # |
| YJZ1031 | **** | **** | # |
| YJZ1025 | *** | *** | # |
| YJZ9173 | *** | *** | # |
| YJZ1065 | *** | *** | # |
| YJZ9171 | * | ** | # |
| YJZ8070 | *** | ** | # |
| YJZ1072 | *** | ** | # |

IC₅₀: <15 nM = *; 15-50 nM = **; 50-500 nM = ***; >500 nM = ****, #: Not tested.

As can be seen from the data in Table 1, the representative compound YJZ5118 of the novel CDK12/13 covalent inhibitor of the present invention has strong inhibitory activities on CDK12/13 kinase, and has good selectivity for CDK9 kinase.

### Example 55: Study on Inhibitory Activity on Cell Proliferation

The VCaP cell proliferation activity was determined by CellTiter-Glo Luminescent Cell Viability Assay (Promega, Madison, WI). The cells were inoculated on a 96-well plate in their respective culture mediums, and incubated in an incubator with 5% CO₂ at 37° C. After incubation overnight, serial dilutions of the compound were prepared, and added into the 96-well plate. A CellTiter-Glo reagent (volume equal to the volume of the cell culture medium present in each well) was added (e.g., 100 μL of the reagent was added into 100 μL of the cell culture medium in the 96-well plate). The contents were mixed on an orbital shaker for 2 min to induce the cell lysis. The cells in the 96-well plate were incubated at room temperature for 10 min, to stabilize the luminescent signal. Luminescence was recorded: the luminescent signal for each well was acquired using an Infinite M1000 Pro plate reader (Tecan, Zurich, Switzerland), and the data was analyzed using GraphPad Prism software (GraphPad Software Inc, La Jolla, CA).

The test result is as shown in Table 2.

TABLE 2
Test Result of Cell Activity of Compound (IC₅₀: nM)

| Compound No. | VCap |
|---|---|
| YJZ5028 | *** |
| YJZ4156 | *** |
| YJZ5111 | ** |
| YJZ5127 | ** |
| YJZ5125 | * |
| YJZ5144 | * |
| YJZ5118 | * |
| YJZ5078 | *** |
| YJZ6077 | *** |
| YJZ6082 | *** |
| YJZ8027 | ** |
| YJZ6093 | * |
| YJZ6028 | ** |
| YJZ6096 | *** |
| YJZ7007 | ** |
| YJZ8053 | *** |
| YJZ8058 | *** |
| YJZ8062 | ** |
| YJZ8070 | * |
| YJZ8103 | ** |
| YJZ8104 | ** |
| YJZ8095 | ** |
| YJZ8105 | ** |
| YJZ8106 | ** |
| YJZ8094 | ** |
| YJZ8087 | ** |
| YJZ8093 | ** |
| YJZ8107 | ** |
| YJZ8101 | * |
| YJZ8102 | ** |
| YJZ6115 | **** |
| YJZ6131 | **** |
| YJZ6136 | *** |

IC₅₀: <15 nM = *; 15-50 nM = **; 50-500 nM = ***; >500 nM = ****.

As can be seen from the data in Table 2, a class of novel CDK12/13 covalent inhibitor compounds of the present invention have very strong inhibitory activities on the VCap cell proliferation.

All documents mentioned herein are incorporated by reference in the present invention, just like each document being individually incorporated by reference. In addition, it should be understood that after reading the above teachings in the present invention, those skilled in the art can make various alterations or modifications to the present invention, and these equivalent forms also fall within the scope defined by the appended claims of the present invention.

1. A compound of formula (I), or a pharmaceutically acceptable salt thereof: 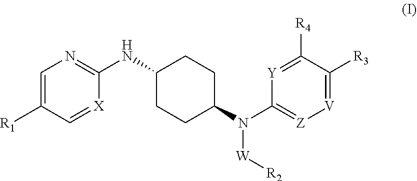
wherein X, Y, and Z are each independently selected from the group consisting of: N or CR₅;
R₅ is selected from the group consisting of: hydrogen, halogen, cyano, hydroxyl, amino, halomethyl, halomethoxy, haloethyl, haloethoxy, C₁-C₆ alkyl, C₁-C₆ alkoxy, C₃-C₈ cycloalkyl, C₃-C₈ cycloalkoxy, or C₁-C₆ alkyl-substituted amino;
R₁ is selected from the group consisting of: H, cyano, halogen, halomethyl, halomethoxy, haloethoxy, haloethyl, C₁-C₆ alkyl, C₃-C₈ cycloalkyl, C₁-C₆ alkoxy, or C₃-C₈ cycloalkoxy;
W is selected from the group consisting of: a chemical bond, 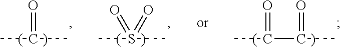
R₂ is selected from the group consisting of: —NHR₆, —OR₆, —CH(R₈)R₆, or H;
R₆ is selected from the group consisting of: —(C(R₈)R₇)R₉ or —(CH₂)ₙR₉; wherein n is selected from: 0, 1, or 2;
R₇ and R₈ are each independently selected from the group consisting of: hydrogen, halogen, cyano, methyl, halomethyl, methoxy, halomethoxy, ethyl, haloethyl, ethoxy, haloethoxy, hydroxyl, amino, or 3-8-membered heterocyclic ring containing m heteroatoms, wherein m is selected from: O, S, or N; or R₇ and R₈ form, together with the C atom to which they are attached, 3-7 heterocyclic rings containing n heteroatoms, wherein n is selected from: 1, 2, or 3, and the heteroatom is selected from: O, N, or S;
R₉ is selected from the group consisting of:
1) cyano, C₁-C₅ alkyl, halogenated C₁-C₄ alkyl, C₁-C₄ alkoxy, C₃-C₁₀ cycloalkyl, substituted or unsubstituted 3-8-membered aromatic ring or saturated ring containing O, S, or N, or 8-12-membered fused ring, spiro ring, or bridged ring containing n heteroatoms; wherein n is selected from: 1, 2, or 3, and the heteroatom is selected from: O, N, or S; or
2) 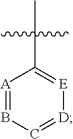
wherein A, B, C, D, and E are each independently selected from the group consisting of: CH, N, or CR₁₀;
R₁₀ is selected from the group consisting of: halogen, cyano, hydroxyl, amino, nitro, C₁-C₃ alkyl, halogenated C₁-C₃ alkyl, C₁-C₄ alkoxy, halogenated C₁-C₄ alkoxy, or C₃-C₈ cycloalkyl;
V is N or CR₃;
each R₃ is independently selected from the group consisting of: H, halogen, cyano, hydroxyl, amino, C₁-C₃ alkyl, halogenated C₁-C₃ alkyl, C₁-C₃ alkoxy, halogenated C₁-C₃ alkoxy, C₃-C₈ cycloalkyl, —(CH₂)ₘR₁₁, —NH(CH₂)ₘR₁₁, —NR₁₄(CH₂)ₘR₁₁, or —O(CH₂)ₘR₁₁; substituted or unsubstituted 3-8-membered heterocyclic ring containing m heteroatoms, wherein m is selected from: 1, 2, and 3, and the heteroatom is selected from: O, S, or N; or 8-12-membered fused ring, spiro ring, or bridged ring containing n heteroatoms, wherein n is selected from: 1, 2, or 3, the heteroatom is selected from: O, N, or S; and at least one R₃ is not H;
R₁₁ is selected from the group consisting of: C₁-C₆ alkyl or NR₁₂R₁₃; wherein R₁₂ and R₁₃ are each independently selected from the group consisting of: H, C₁-C₈ alkyl, —(CH₂)ₘNR₁₄R₁₅, or —(CH₂)ₙCR₁₄R₁₅R₁₆, or R₁₂ and R₁₃ form, together with a nitrogen atom to which they are attached, a substituted or unsubstituted heteroatom-containing monocyclic ring, fused ring, spiro ring, or bridged ring;
R₁₄, R₁₅, and R₁₆ are each independently selected from the group consisting of: H or C₁-C₈ alkyl, or R₁₄ and R₁₅ form, together with a nitrogen atom or carbon atom to which they are attached, a substituted or unsubstituted monocyclic ring, fused ring, spiro ring, or bridged ring containing from 0 to 3 heteroatoms;
m and n are independently selected from: an integer between 0 and 8;
R₄ is selected from the group consisting of: H, 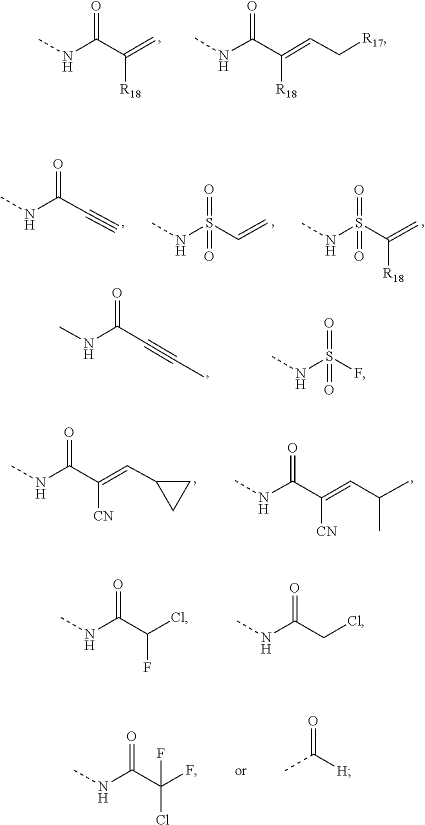
R₁₇ is selected from the group consisting of: hydrogen, trifluoromethyl, 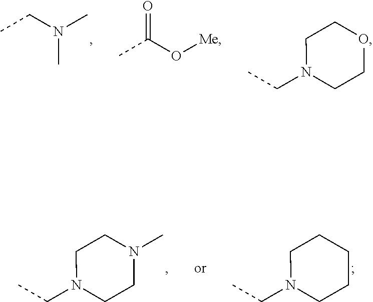
R₁₈ is selected from the group consisting of: hydrogen or fluorine; and
unless otherwise specified, the “substituted” refers to the replacement of one or more hydrogen atoms on a group with a substituent selected from the group consisting of: halogen, oxo, unsubstituted or halogenated C1-C6 alkyl, unsubstituted or halogenated C2-C6 alkenyl, unsubstituted or halogenated C2-C6 alkynyl, unsubstituted or halogenated C1-C6 alkoxy, unsubstituted or halogenated C1-C6 acyl, unsubstituted or halogenated C1-C6 amido, unsubstituted or halogenated C1-C6 alkylamino, unsubstituted or halogenated C1-C6 alkyl-hydroxyl, unsubstituted or halogenated C3-C6 alkyl, or unsubstituted or halogenated 4-8-membered heterocyclyl.

2. The compound of claim 1, or a pharmaceutically acceptable salt thereof, wherein:
X, Y, and Z are each independently selected from: N or CR₅;
R₅ is selected from the group consisting of: hydrogen, halogen, cyano, halomethyl, or halomethoxy.

3. The compound of claim 1, or a pharmaceutically acceptable salt thereof, wherein:
R₁ is selected from the group consisting of: H, cyano, halogen, halomethyl, halomethoxy, haloethoxy, haloethyl, C₁-C₆ alkyl, C₃-C₈ cycloalkyl, or C₁-C₆ alkoxy.

4. The compound of claim 1, or a pharmaceutically acceptable salt thereof, wherein:
W is selected from the group consisting of:

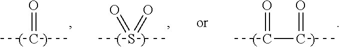

5. The compound of claim 1, or a pharmaceutically acceptable salt thereof, wherein:
R₂ is selected from the group consisting of: —NHR₆;
R₆ is selected from the group consisting of: —(C(R₈)R₇)R₉;
R₇ and R₈ are each independently selected from the group consisting of: hydrogen, halogen, cyano, methyl, halomethyl, methoxy, or halomethoxy; and
R₉ is each independently selected from the group consisting of: phenyl, halophenyl, pyridyl, pyrimidyl, isopropyl, tert-butyl, trifluoromethyl, difluoromethyl, cyano, pyrrolyl, N-methylpyrrolyl, N-methylimidazolyl, N-methylpyrazolyl, imidazolyl, furyl, thienyl, pyrazolyl, isoxazolyl, oxazolyl, halogenated C₁-C₄ alkyl, C₁-C₄ alkoxy, C₁-C₄ alkyl, C₃-C₇ cycloalkyl, or C₃-C₇ epoxyalkyl.

6. The compound of claim 1, or a pharmaceutically acceptable salt thereof, wherein:
each R₃ is independently selected from the group consisting of: H, halogen, cyano, hydroxyl, amino, C₁-C₃ alkyl, halogenated C₁-C₃ alkyl, C₁-C₃ alkoxy, halogenated C₁-C₃ alkoxy, C₃-C₈ cycloalkyl, C₃-C₈ cycloalkoxy,

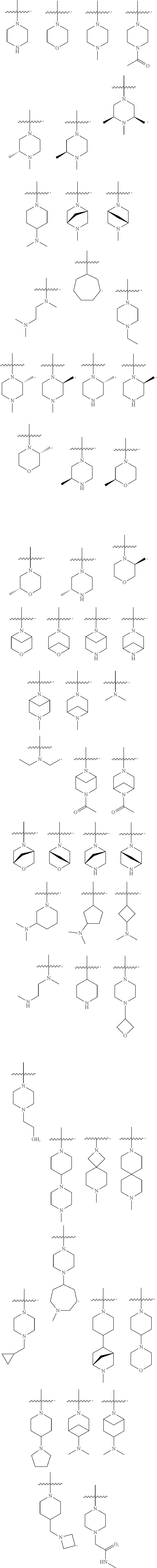

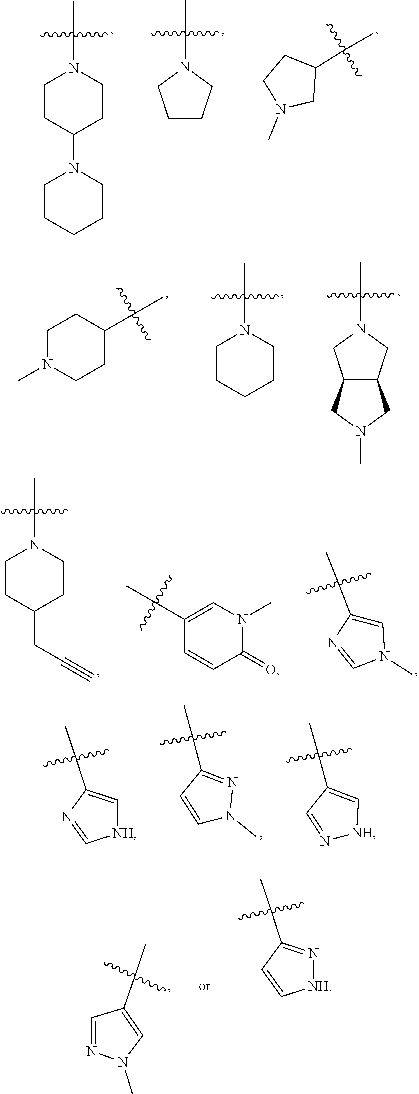

7. The compound of claim 1, or a pharmaceutically acceptable salt thereof, wherein:
R₄ is selected from the group consisting of: H,

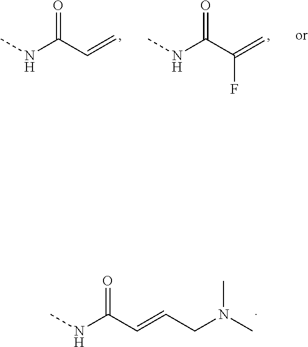

8. The compound of claim 1, or a pharmaceutically acceptable salt thereof, having a structure shown in formula (II) or (III):

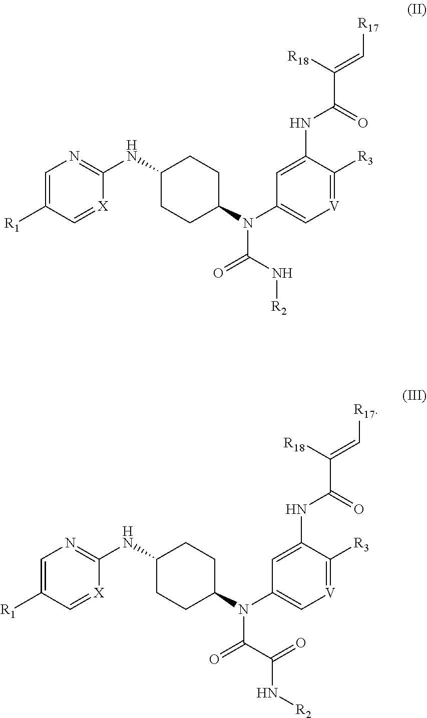

9. The compound of claim 1, wherein the compound is selected from the group consisting of:
N-(5-(3-benzyl-1-((1r,4r)-4-((5-cyanopyridin-2-yl)amino)cyclohexyl)ureido)-2-(4-(dimethylamino)piperidin-1-yl)phenyl)acrylamide;
N-(2-(4-acetylpiperazin-1-yl)-5-(3-benzyl-1-((1r,4r)-4-((5-cyanopyridin-2-yl)amino)cyclohexyl)ureido)phenyl)acrylamide;
N-(5-(3-benzyl-1-((1r,4r)-4-((5-cyanopyridin-2-yl)amino)cyclohexyl)ureido)-2-(4-methylpiperazin-1-yl)phenyl)acrylamide;
N-(5-(3-benzyl-1-((1r,4r)-4-((5-cyanopyridin-2-yl)amino)cyclohexyl)ureido)-2-morpholinophenyl)acrylamide;
N-(5-(3-benzyl-1-((1r,4r)-4-((5-cyanopyridin-2-yl)amino)cyclohexyl)ureido)-2-((2-(dimethylamino)ethyl)(methyl)amino)phenyl)acrylamide;
N-(5-(3-benzyl-1-((1r,4R)-4-((5-cyanopyridin-2-yl)amino)cyclohexyl)ureido)-2-((1R,4R)-5-methyl-2,5-diazabicyclo[2.2.1]heptan-2-yl)phenyl)acrylamide;
N-(5-(3-benzyl-1-((1r,4r)-4-((5-cyanopyridin-2-yl)amino)cyclohexyl)ureido)-2-(4-(oxetan-3-yl)piperazin-1-yl)phenyl)acrylamide;
N-(5-(3-benzyl-1-((1r,4r)-4-((5-cyanopyridin-2-yl)amino)cyclohexyl)ureido)-2-(4-(prop-2-yn-1-yl)piperazin-1-yl)phenyl)acrylamide;
N-(5-(3-benzyl-1-((1r,4r)-4-((5-cyanopyridin-2-yl)amino)cyclohexyl)ureido)-2-(3-(dimethylamino)pyrrolidin-1-yl)phenyl)acrylamide;
N-(5-(3-benzyl-1-((1r,4r)-4-((5-cyanopyridin-2-yl)amino)cyclohexyl)ureido)-2-(3-(dimethylamino)azetidin-1-yl)phenyl)acrylamide;
N-(5-(3-benzyl-1-((1r,4r)-4-((5-cyanopyridin-2-yl)amino)cyclohexyl)ureido)-2-bromophenyl)acrylamide;
N-(5-(3-benzyl-1-((1r,4S)-4-((5-cyanopyridin-2-yl)amino)cyclohexyl)ureido)-2-((S)-3,4-dimethylpiperazin-1-yl)phenyl)acrylamide;
N-(5-(3-benzyl-1-((1r,4S)-4-((5-cyanopyridin-2-yl)amino)cyclohexyl)ureido)-2-((S)-3-methylpiperazin-1-yl)phenyl)acrylamide;
N-(5-(3-benzyl-1-((1r,4r)-4-((5-cyanopyridin-2-yl)amino)cyclohexyl)ureido)-2-(1-methyl-6-oxo-1,6-dihydropyridin-3-yl)phenyl)acrylamide;
N-(3-(3-benzyl-1-((1r,4r)-4-((5-cyanopyridin-2-yl)amino)cyclohexyl)ureido)phenyl)acrylamide;
(E)-N-(5-(3-benzyl-1-((1r,4r)-4-((5-cyanopyridin-2-yl)amino)cyclohexyl)ureido)-2-(1-methyl-6-oxo-1,6-dihydropyridin-3-yl)phenyl)-4-(dimethylamino)but-2-enamide;
N-(5-(3-benzyl-1-((1r,4S)-4-((5-cyanopyrimidin-2-yl)amino)cyclohexyl)ureido)-2-((S)-3,4-dimethylpiperazin-1-yl)phenyl)acrylamide;
N-(5-((((1r,4r)-4-)((5-cyanopyridin-2-yl)amino)cyclohexyl)amino)-2-(4-(dimethylamino)piperidin-1-yl)phenyl)acrylamide;
N-(5-(1-((1r,4S)-4-((5-cyanopyridin-2-yl)amino)cyclohexyl)-3-(cyclopropylmethyl)ureido)-2-((S)-3,4-dimethylpiperazin-1-yl)phenyl)acrylamide;
N-(5-(1-((1r,4S)-4-((5-cyanopyridin-2-yl)amino)cyclohexyl)-3-(oxetanyl-3-ylmethyl)ureido)-2-((S)-3,4-dimethylpiperazin-1-yl)phenyl)acrylamide;
N-(5-(1-((1r,4S)-4-((5-cyanopyridin-2-yl)amino)cyclohexyl)-3-((1-methyl-1H-pyrazol-4-yl)methyl)ureido)-2-((S)-3,4-dimethylpiperazin-1-yl)phenyl)acrylamide;
N-(5-(1-((1r,4S)-4-((5-cyanopyridin-2-yl)amino)cyclohexyl)-3-cyclopropylureido)-2-((S)-3,4-dimethylpiperazin-1-yl)phenyl)acrylamide;
N-(5-(1-((1r,4S)-4-((5-cyanopyridin-2-yl)amino)cyclohexyl)-3-(2-fluorobenzyl)ureido)-2-((S)-3,4-dimethylpiperazin-1-yl)phenyl)acrylamide;
N-(5-(1-((1r,4S)-4-((5-cyanopyridin-2-yl)amino)cyclohexyl)-3-(3-fluorobenzyl)ureido)-2-((S)-3,4-dimethylpiperazin-1-yl)phenyl)acrylamide;
N-(5-(1-((1r,4S)-4-((5-cyanopyridin-2-yl)amino)cyclohexyl)-3-(4-fluorobenzyl)ureido)-2-((S)-3,4-dimethylpiperazin-1-yl)phenyl)acrylamide;
N-(5-(1-((1r,4S)-4-((5-cyanopyridin-2-yl)amino)cyclohexyl)-3-((S)-1-phenylethyl)ureido)-2-((S)-3,4-dimethylpiperazin-1-yl)phenyl)acrylamide;
N-(5-(1-((1r,4R)-4-((5-cyanopyridin-2-yl)amino)cyclohexyl)-3-((R)-1-phenylethyl)ureido)-2-((S)-3,4-dimethylpiperazin-1-yl)phenyl)acrylamide;
N-(5-(N-((1r,4r)-4-((5-cyanopyridin-2-yl)amino)cyclohexyl)methylsulfonamido)-2-(4-(dimethylamino)piperidin-1-yl)phenyl)acrylamide;
N-(5-((N-((1r,4r)-4-((5-cyanopyridin-2-yl)amino)cyclohexyl)-1-phenylmethyl)sulfonamido)-2-(4-(dimethylamino)piperidin-1-yl)phenyl)acrylamide;
N-(5-(N-((1r,4r)-4-((5-cyanopyridin-2-yl)amino)cyclohexyl)acetamido)-2-(4-(dimethylamino)piperidin-1-yl)phenyl)acrylamide;
N-(5-(3-benzyl-1-((1r,4S)-4-((5-cyanopyridin-2-yl)amino)cyclohexyl)ureido)-2-((S)-3,4-dimethylpiperazin-1-yl)-3-fluorophenyl)acrylamide;
N-(5-(3-benzyl-1-((1r,4S)-4-((5-cyanopyridin-2-yl)amino)cyclohexyl)ureido)-2-((S)-3,4-dimethylpiperazin-1-yl)pyridin-3-yl)acrylamide;
N-(3-(3-benzyl-1-((1r,4r)-4-((5-cyanopyridin-2-yl)amino)cyclohexyl)ureido)-5-bromophenyl)acrylamide;
N-(3-(3-benzyl-1-((1r,4r)-4-((5-cyanopyridin-2-yl)amino)cyclohexyl)ureido)-5-methoxyphenyl)acrylamide;
N-(3-(3-benzyl-1-((1r,4S)-4-((5-cyanopyridin-2-yl)amino)cyclohexyl)ureido)-5-((S)-3,4-dimethylpiperazin-1-yl)phenyl)acrylamide;
3-benzyl-1-((1r,4r)-4-((5-cyanopyridin-2-yl)amino)cyclohexyl)-1-(4-(piperazin-1-yl)phenyl)urea;
N1-(3-acrylamido-4-((S)-3,4-dimethylpiperazin-1-yl)phenyl)-N1-((1r,4S)-4-((5-cyanopyridin-2-yl)amino)cyclohexyl)-N2-(3,4-difluorobenzyl)oxalamide;
N1-(3-acrylamido-4-((S)-3,4-dimethylpiperazin-1-yl)phenyl)-N1-((1r,4S)-4-((5-cyanopyridin-2-yl)amino)cyclohexyl)-N2-(3-fluorobenzyl)oxalamide;
N1-(3-acrylamido-4-((S)-3,4-dimethylpiperazin-1-yl)phenyl)-N1-((1r,4S)-4-((5-cyanopyridin-2-yl)amino)cyclohexyl)-N2-(4-fluorobenzyl)oxalamide;
N1-(3-acrylamido-4-((S)-3,4-dimethylpiperazin-1-yl)phenyl)-N1-((1r,4S)-4-((5-cyanopyridin-2-yl)amino)cyclohexyl)-N2-(2,3-difluorobenzyl)oxalamide;
N1-(3-acrylamido-4-((S)-3,4-dimethylpiperazin-1-yl)phenyl)-N1-((1r,4S)-4-((5-cyanopyridin-2-yl)amino)cyclohexyl)-N2-(3,5-difluorobenzyl)oxalamide;
N1-(3-acrylamido-4-((S)-3,4-dimethylpiperazin-1-yl)phenyl)-N1-((1r,4R)-4-((5-cyanopyridin-2-yl)amino)cyclohexyl)-N2-((R)-1-phenylethyl)oxalamide;
N1-(3-acrylamido-4-((S)-3,4-dimethylpiperazin-1-yl)phenyl)-N1-((1r,4S)-4-((5-cyanopyridin-2-yl)amino)cyclohexyl)-N2-((S)-1-phenylethyl)oxalamide;
N1-(3-acrylamido-4-((S)-3,4-dimethylpiperazin-1-yl)phenyl)-N1-((1r,4S)-4-((5-cyanopyridin-2-yl)amino)cyclohexyl)-N2-(cyclopropylmethyl)oxalamide;
N1-(3-acrylamido-4-((S)-3,4-dimethylpiperazin-1-yl)phenyl)-N1-((1r,4S)-4-((5-cyanopyridin-2-yl)amino)cyclohexyl)-N2-cyclopropyloxalamide;
N1-(3-acrylamido-4-((S)-3,4-dimethylpiperazin-1-yl)phenyl)-N1-((1r,4S)-4-((5-cyanopyridin-2-yl)amino)cyclohexyl)-N2-(2,2,2-trifluoroethyl)oxalamide;
N-(5-(3-benzyl-1-((1r,4S)-4-((5-cyanopyridin-2-yl)amino)cyclohexyl)ureido)-2-((S)-3,4-dimethylpiperazin-1-yl)phenyl)-2-fluoroacrylamide;
N-(5-(3-benzyl-1-((1r,4S)-4-((5-(trifluoromethyl)pyridin-2-yl)amino)cyclohexyl)ureido)-2-((S)-3,4-dimethylpiperazin-1-yl)phenyl)acrylamide;
N-(5-(1-((1r,4S)-4-((5-cyanopyridin-2-yl)amino)cyclohexyl)-3-(2,2,2-trifluoroethyl)ureido)-2-((S)-3,4-dimethylpiperazin-1-yl)phenyl)acrylamide;
N-(5-(1-((1r,4S)-4-((5-cyanopyridin-2-yl)amino)cyclohexyl)-3-(oxazol-4-ylmethyl)ureido)-2-((S)-3,4-dimethylpiperazin-1-yl)phenyl)acrylamide;
N-(5-(1-((1r,4S)-4-((5-cyanopyridin-2-yl)amino)cyclohexyl)-3-(pyridin-3-ylmethyl)ureido)-2-((S)-3,4-dimethylpiperazin-1-yl)phenyl)acrylamide;
N-(5-(3-(2-chlorobenzyl)-1-((1r,4S)-4-((5-cyanopyridin-2-yl)amino)cyclohexyl)ureido)-2-((S)-3,4-dimethylpiperazin-1-yl)phenyl)acrylamide; and
N-(5-(3-benzyl-1-((1r,4S)-4-((5-chloropyridin-2-yl)amino)cyclohexyl)ureido)-2-((S)-3,4-dimethylpiperazin-1-yl)phenyl)acrylamide,
and pharmaceutically acceptable salts thereof.

10. A pharmaceutical composition comprising the compound of claim 1, or a pharmaceutically acceptable salt thereof, and a pharmaceutically acceptable carrier.

11. A method of treating a disease mediated by a CDK12/13 serine/threonine protein kinase in a subject in need thereof, comprising administering to the subject a therapeutically effective amount of a compound of claim 1, or a pharmaceutically acceptable salt thereof.

12. The method of claim 11, wherein the disease mediated by the CDK12/13 serine/threonine protein kinase is selected from the group consisting of: prostate cancer, breast cancer, uterine cancer, ovarian cancer, non-small cell lung cancer, small cell lung cancer, Ewing sarcoma, lung adenocarcinoma, squamous cell lung carcinoma, pancreatic cancer, liver cancer, skin cancer, epithelial cell carcinoma, gastrointestinal stromal tumor, leukemia, histiocytic lymphoma, nasopharyngeal carcinoma, head and neck tumor, colon cancer, rectal cancer, and glioma.
